(19) United States
(12) Patent Application Publication
(10) Pub. No.: US 2024/0051962 A1
(43) Pub. Date: Feb. 15, 2024

(54) SOLID FORMS OF A NUCLEOSIDE ANALOGUE AND USES THEREOF

(71) Applicant: Gilead Sciences, Inc., Foster City, CA (US)

(72) Inventors: Kassibla E. Dempah, San Francisco, CA (US); Stefani Kocevska, Foster City, CA (US); Chiajen Lai, Livermore, CA (US)

(21) Appl. No.: 18/215,217

(22) Filed: Jun. 28, 2023

Related U.S. Application Data
(60) Provisional application No. 63/356,899, filed on Jun. 29, 2022.

(51) Int. Cl. C07D 487/04

(52) U.S. Cl. CPC C07D 487/04; C07B 2200/13

(57) ABSTRACT

The disclosure provides solid forms, e.g. salts and polymorphs, of the compound of Formula (I) and uses thereof. Also provided are the methods of making the solid forms and pharmaceutical formulations comprising the solid forms.

### CROSS REFERENCE

This application claims priority to U.S. Provisional Application No. 63/356,899 filed Jun. 29, 2022. The entire contents of which application are incorporated herein in their entirety for all purposes.

### TECHNICAL FIELD

The present disclosure relates to solid forms of ((2R,3S,4R,5R)-5-(4-aminopyrrolo[2,1-f][1,2,4]triazin-7-yl)-5-cyano-3,4-dihydroxytetrahydrofuran-2-yl)methyl isopropyl carbonate, for use in the treatment of a viral infections. The present disclosure also relates to pharmaceutical compositions containing the solid forms disclosed herein, and methods of treating or preventing viral infections.

### BACKGROUND

There is an ongoing need for antiviral agents and methods for treating viral infections, for example paramyxoviridae, pneumoviridae, picornaviridae, flaviviridae, filoviridae, arenaviridae, orthomyxovirus, and coronaviridae infections. There is also a constant need to develop methods for preparation and purification of the antiviral agents, as well as prepare improved pharmaceutical formulations of the same. The solid forms disclosed herein help meet these and other needs.

### SUMMARY

The present application provides solid forms of the compound of Formula (I):

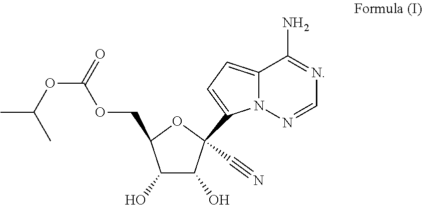 [SMILES: CC(C)OC(=O)OC[C@H]1O[C@@](C#N)(c2ccc3c(N)ncnn23)[C@H](O)[C@@H]1O]

The present application further provides crystalline forms of the compound of Formula (I), including solvates and hydrates and crystalline forms thereof.

The present application further provides methods of making the solid forms disclosed herein.

The present application further provides a pharmaceutical composition comprising the solid forms disclosed herein and a pharmaceutically acceptable excipient.

The present application further provides a kit comprising the solid forms disclosed herein and a pharmaceutically acceptable excipient.

The present application further provides a method of treating or preventing a viral infection in a human in need thereof, wherein the method comprises administering to the human the solid forms or the pharmaceutical compositions disclosed herein.

The present application further provides a method for manufacturing a medicament for treating or preventing a viral infection in a human in need thereof, characterized in that a solid form or pharmaceutical compositions disclosed herein is used.

The present application further provides use of the solid forms or pharmaceutical compositions disclosed herein for the manufacture of a medicament for the treatment or prevention of a viral infection in a human in need thereof.

The present application further provides the solid forms or pharmaceutical compositions disclosed herein for use in treatment or prevention of a viral infection in a human in need thereof.

### BRIEF DESCRIPTION OF THE DRAWINGS

FIG. 1. Shows the XRPD pattern of compound of Formula (I) hydrate Form H1.

FIG. 2. Shows the XRPD pattern of compound of Formula (I) Form I.

FIG. 3. Shows the DSC thermogram of compound of Formula (I) Form I.

FIG. 4. Shows the TGA thermogram of compound of Formula (I) Form I.

FIG. 5. Shows the XRPD pattern of compound of Formula (I) acetonitrile solvate Form ACN1.

FIG. 6. Shows the XRPD pattern of compound of Formula (I) acetonitrile solvate Form ACN2.

FIG. 7. Shows the XRPD pattern of compound of Formula (I) acetonitrile solvate Form ACN3.

FIG. 8. Shows the XRPD pattern of compound of Formula (I) Form II.

FIG. 9. Shows the DSC thermogram of compound of Formula (I) Form II.

FIG. 10. Shows the TGA thermogram of compound of Formula (I) Form II.

FIG. 11. Shows the XRPD pattern of compound of Formula (I), HCl salt, Form I.

FIG. 12. Shows the DSC thermogram of compound of Formula (I), HCl salt, Form I.

FIG. 13. Shows the TGA thermogram of compound of Formula (I), HCl salt, Form I.

FIG. 14. Shows the XRPD pattern of compound of Formula (I), oxalate salt, Form I.

FIG. 15. Shows the DSC thermogram of compound of Formula (I), oxalate salt, Form I.

FIG. 16. Shows the TGA thermogram of compound of Formula (I), oxalate salt, Form I.

FIG. 17. Shows the XRPD pattern of compound of Formula (I), oxalate salt, Form II.

FIG. 18. Shows the DSC thermogram of compound of Formula (I), oxalate salt, Form II.

FIG. 19. Shows the TGA thermogram of compound of Formula (I), oxalate salt, Form II.

FIG. 20. Shows the XRPD pattern of compound of Formula (I), maleate salt, Form I.

FIG. 21. Shows the XRPD pattern of compound of Formula (I), maleate salt, Form II.

FIG. 22. Shows the XRPD pattern of compound of Formula (I), maleate salt, Form III.

FIG. 23. Shows the DSC thermogram of compound of Formula (I), maleate salt, Form III.

FIG. 24. Shows the TGA thermogram of compound of Formula (I), maleate salt, Form III.

FIG. 25. Shows the XRPD pattern of compound of Formula (I), maleate salt, Form IV.

FIG. 26. Shows the DSC thermogram of compound of Formula (I), maleate salt, Form IV.

FIG. 27. Shows the TGA thermogram of compound of Formula (I), maleate salt, Form IV.

FIG. 28. Shows the XRPD pattern of compound of Formula (I), maleate salt, MeCN solvate I.

FIG. 29. Shows the XRPD pattern of compound of Formula (I), maleate salt, MeCN solvate II.

FIG. 30. Shows the XRPD pattern of compound of Formula (I), mesylate salt, IPA solvate.

FIG. 31. Shows the XRPD pattern of compound of Formula (I), mesylate salt, hydrate.

FIG. 32. Shows the DSC thermogram of compound of Formula (I), mesylate salt, hydrate.

FIG. 33. Shows the TGA thermogram of compound of Formula (I), mesylate salt, hydrate.

FIG. 34. Shows the XRPD pattern of compound of Formula (I), mesylate salt, Form I.

FIG. 35. Shows the DSC thermogram of compound of Formula (I), mesylate salt, Form I.

FIG. 36. Shows the TGA thermogram of compound of Formula (I), mesylate salt, Form I.

FIG. 37. Shows the XRPD pattern of compound of Formula (I), Form III.

FIG. 38. Shows the DSC thermogram of compound of Formula (I), Form IV.

FIG. 39. Shows the TGA thermogram of compound of Formula (I), Form IV.

FIG. 40. Shows the XRPD pattern of compound of Formula (I), Form IV.

FIG. 41. Shows the DSC thermogram of compound of Formula (I), Form IV.

FIG. 42. Shows the TGA thermogram of compound of Formula (I), Form IV.

### DETAILED DESCRIPTION

The present disclosure relates to new solid forms of ((2R,3S,4R,5R)-5-(4-aminopyrrolo[2,1-f][1,2,4]triazin-7-yl)-5-cyano-3,4-dihydroxytetrahydrofuran-2-yl)methyl isopropyl carbonate (i.e. the compound of Formula (I), see below). One skilled in the art understands that a compound structure may be named or identified using commonly recognized nomenclature systems and symbols. By way of example, the compound may be named or identified with common names, systematic or non-systematic names. The nomenclature systems and symbols that are commonly recognized in the art of chemistry including but not limited to Chemical Abstract Service (CAS) and International Union of Pure and Applied Chemistry (IUPAC).

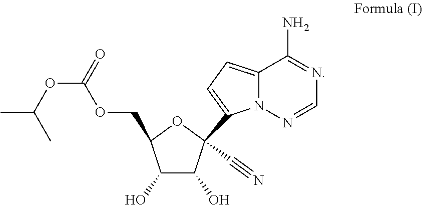 [SMILES: CC(C)OC(=O)OC[C@H]1O[C@@](C#N)(c2ccc3c(N)ncnn23)[C@H](O)[C@@H]1O]

The solid forms of the invention include salt forms (both amorphous and crystalline) as well as cocrystal forms of the compound of Formula (I). As used herein, “solid form” generally refers to a solid chemical substance that can be amorphous or crystalline. In some embodiments, the solid form of the invention is a salt of compound of Formula (I) which can be amorphous or crystalline. In further embodiments, the solid form can be a cocrystal of compound of Formula (I), in which compound of Formula (I) has formed a crystalline solid together with a coformer molecule. In some embodiments, the solid form is a solvate (e.g. a hydrate). Both crystalline salts and cocrystals of compound of Formula (I) can exist in different crystalline forms (i.e., have different polymorphic or pseudopolymorphic forms).

As used herein, the term “cocrystal” refers to a compound (such as compound of Formula (I)) crystallized together with one or more coformer molecules (e.g., molecules other than the compound). Depending on the chemical nature and proportion of coformers present in the cocrystal, different physical properties related to, for example, dissolution and solubility may be observed compared with solid forms of the compound by itself or salts thereof. In some instances, the coformer molecule may be a protic acid, and whether the protic acid forms a salt or a cocrystal will often depend on the relative pKa's of the compound and coformer. See, e.g., Regulatory Classification of Pharmaceutical Co-Crystals: Guidance for Industry, revised August 2016, published by the U.S. Dept. of Health and Human Services, FDA, Center for Drug Evaluation and Research (CDER).

As used herein, “crystalline form” is meant to refer to a certain lattice configuration of a crystalline substance (e.g., a salt or a cocrystal). Different crystalline forms of the same substance typically have different crystalline lattices (e.g., unit cells) which are attributed to different physical properties that are characteristic of each of the crystalline forms. In some instances, different lattice configurations have different water or solvent content.

According to the present invention, a crystalline forms of compound of Formula (I) can be useful in the synthesis and/or purification of the compound of Formula (I). For example, a crystalline form of compound of Formula (I) can be an intermediate in the synthesis of the compound 1 of Formula (I). In addition, different crystalline forms of compound of Formula (I) may have different properties with respect to bioavailability, stability, purity, and/or manufacturability for medical or pharmaceutical uses. Variations in the crystal structure of a pharmaceutical drug substance or active ingredient may affect the dissolution rate (which may affect bioavailability, etc.), manufacturability (e.g., ease of handling, ability to consistently prepare doses of known strength), and stability (e.g., thermal stability, shelf life, etc.) of a pharmaceutical drug product or active ingredient. Such variations may affect the preparation or formulation of pharmaceutical compositions in different dosage or delivery forms, such as solutions or solid oral dosage form including tablets and capsules. Compared to other forms such as non-crystalline or amorphous forms, crystalline forms may provide desired or suitable hygroscopicity, particle size controls, dissolution rate, solubility, purity, physical and chemical stability, manufacturability, yield, and/or process control. Thus, the crystalline forms of compound of Formula (I) may provide advantages such as improving the manufacturing process of the compound, the stability or storability of a drug product form of the compound, the stability or storability of a drug substance of the compound and/or the bioavailability and/or stability of the compound as an active agent.

The use of certain solvents and/or processes have been found to produce different crystalline forms of compound of Formula (I) which may exhibit one or more of the favorable characteristics described above. The processes for the preparation of the crystalline forms described herein and characterization of these crystalline and cocrystal forms are described in detail below.

In some embodiments, the crystalline forms described herein are purified or substantially isolated. By “substantially isolated” is meant that the crystalline form is at least partially or substantially separated from the environment in which it was formed or detected. Partial separation can include, for example, a composition enriched in the crystalline form of the disclosure. Substantial separation can include compositions containing at least about 50%, at least about 60%, at least about 70%, at least about 80%, at least about 90%, at least about 95%, at least about 97%, or at least about 99% by weight of the crystalline form of the disclosure. In some embodiments, the crystalline form of the disclosure can be prepared with a purity of about 75% or more, 80% or more, 85% or more, 90% or more, 95% or more, 98% or more, or 99% or more.

The different crystalline forms can be identified by solid state characterization methods such as by X-ray powder diffraction (XRPD). Other characterization methods such as differential scanning calorimetry (DSC) and thermogravimetric analysis (TGA) further help identify the form as well as help determine stability and solvent/water content.

An XRPD pattern of reflections (peaks) is typically considered a fingerprint of a particular crystalline form. It is well known that the relative intensities of the XRPD peaks can widely vary depending on, inter alia, the sample preparation technique, crystal size distribution, various filters used, the sample mounting procedure, and the particular instrument employed. In some instances, new peaks may be observed or existing peaks may disappear, depending on the type of the instrument or the settings. As used herein, the term “peak” refers to a reflection having a relative height/intensity of at least about 5% of the maximum peak height/intensity. Moreover, instrument variation and other factors can affect the 20 values. Thus, peak assignments, such as those reported herein, can vary by plus or minus about 0.2° (20), and the term “substantially” and “about” as used in the context of XRPD herein is meant to encompass the above-mentioned variations.

In the same way, temperature readings in connection with DSC can vary about ±3° C. depending on the instrument, particular settings, sample preparation, etc. Accordingly, a crystalline form reported herein having a DSC thermogram “substantially” as shown in any of the Figures or the term “about” is understood to accommodate such variation.

The present invention provides crystalline forms of the compound of Formula (I). In some embodiments, the crystalline form may be substantially anhydrous. In some embodiments, the crystalline form may be hydrated or solvated.

Compound of Formula (I) hydrate Form H1

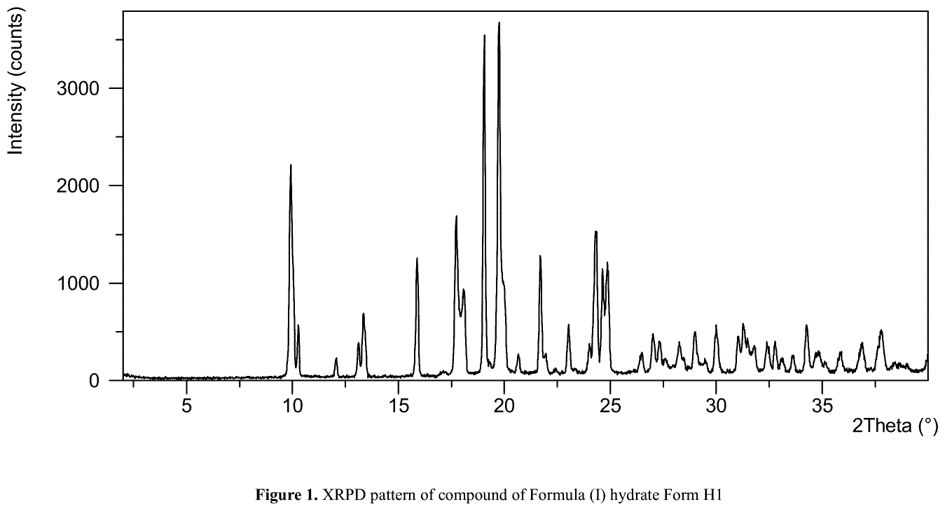

In some embodiments, the present disclosure provides crystalline form of the compound of Formula (I). In some embodiments, the crystalline form is hydrated. In some embodiments, the crystalline form is hydrated Form H1 (“hydrated crystalline Form H1”). In some embodiments, hydrated crystalline Form H1 has an XRPD profile substantially as shown in FIG. 1. In some embodiments, hydrated crystalline Form H1 has an XRPD pattern displaying at least two, at least three, at least four, at least five, or at least six of the 2θ-reflections as the XRPD pattern substantially as shown in FIG. 1.

In some embodiments, the hydrated crystalline Form H1 is characterized by an XRPD pattern comprising 2θ-reflections (±0.2 degrees 2θ) at about 9.9°, 15.9°, and 19.7°. In some embodiments, In some embodiments, the hydrated crystalline Form H1 has an XRPD pattern comprising 2θ-reflections (±0.2 degrees 2θ) at 9.9°, 15.9°, and 19.7°, and one, two or three of the 2θ-reflections (±0.2 degrees 2θ) at 13.3°, 19.0°, and 21.7°. In some embodiments, the hydrated crystalline Form H1 has an XRPD pattern comprising 2θ-reflections (±0.2 degrees 2θ) at 9.9°, 15.9°, and 19.7°, and one or two of the 2θ-reflections (±0.2 degrees 2θ) at 13.3°, 19.0°, and 21.7°. In some embodiments, the hydrated crystalline Form H1 has an XRPD pattern comprising 2θ-reflections (±0.2 degrees 2θ) at 9.9°, 15.9°, and 19.7°, and one of the 20-reflections (±0.2 degrees 2θ) at 13.3°, 19.0°, and 21.7°. In some embodiments, the hydrated crystalline Form H1 has an XRPD pattern comprising 2θ-reflections (±0.2 degrees 2θ) at 9.9°, 15.9°, and 19.7°, and two of the 2θ-reflections (±0.2 degrees 2θ) at 13.3°, 19.0°, and 21.7°. In some embodiments, the hydrated crystalline Form H1 has an XRPD pattern comprising 2θ-reflections (±0.2 degrees 2θ) at 9.9°, 13.3°, 15.9°, 19.0°, 19.7°, and 21.7°. In some embodiments, the hydrated crystalline Form H1 has an XRPD pattern comprising any three 2θ-reflections (±0.2 degrees 2θ) selected from the group consisting of 9.9°, 13.3°, 15.9°, 19.0°, 19.7°, and 21.7°.

In some embodiments, the hydrated crystalline Form H1 has an XRPD pattern comprising 2θ-reflections (±0.2 degrees 2θ) at 9.9°, 13.3°, 15.9°, 19.0°, 19.7°, and 21.7°, and one, two, or three of the 2θ-reflections (±0.2 degrees 2θ) at 17.7°, 12.1°, and 24.4°. In some embodiments, the hydrated crystalline Form H1 has an XRPD pattern comprising 2θ-reflections (±0.2 degrees 2θ) at 9.9°, 13.3°, 15.9°, 19.0°, 19.7°, and 21.7°, and one or two of the 2θ-reflections (±0.2 degrees 2θ) at 17.7°, 12.1°, and 24.4°. In some embodiments, the hydrated crystalline Form H1 has an XRPD pattern comprising 2θ-reflections (±0.2 degrees 2θ) at 9.9°, 13.3°, 15.9°, 19.0°, 19.7°, and 21.7°, and one of the 2θ-reflections (±0.2 degrees 2θ) at 17.7°, 12.1°, and 24.4°. In some embodiments, the hydrated crystalline Form H1 has an XRPD pattern comprising 2θ-reflections (±0.2 degrees 2θ) at 9.9°, 13.3°, 15.9°, 19.0°, 19.7°, and 21.7°, and two of the 2θ-reflections (±0.2 degrees 2θ) at 17.7°, 12.1°, and 24.4°. In some embodiments, the hydrated crystalline Form H1 has an XRPD pattern comprising 2θ-reflections (±0.2 degrees 2θ) at 9.9°, 12.1°, 13.3°, 15.9°, 17.7°, 19.0°, 19.7°, 21.7°, and 24.4°. In some embodiments, the hydrated crystalline Form H1 has an XRPD pattern comprising three of the 2θ-reflections (±0.2 degrees 2θ) at 9.9°, 12.1°, 13.3°, 15.9°, 17.7°, 19.0°, 19.7°, 21.7°, and 24.4°.

In some embodiments, the hydrated crystalline Form H1 has a XRPD pattern comprising peaks at:

| Pos. [°2θ] | Rel. Int. [%] |
|---|---|
| 9.9 | 61 |
| 10.1 | 24 |
| 10.3 | 14 |
| 12.1 | 5 |
| 13.1 | 10 |
| 13.3 | 18 |
| 15.9 | 33 |
| 17.1 | 1 |
| 17.7 | 44 |
| 18.1 | 20 |
| 19.0 | 94 |
| 19.7 | 100 |
| 20.0 | 17 |
| 20.7 | 5 |
| 21.7 | 34 |
| 21.9 | 6 |
| 23.0 | 13 |
| 24.0 | 6 |
| 24.4 | 36 |
| 24.6 | 29 |
| 24.9 | 28 |
| 26.5 | 5 |
| 27.0 | 10 |
| 27.3 | 8 |
| 27.6 | 3 |
| 28.3 | 7 |
| 29.0 | 10 |
| 29.5 | 2 |
| 30.0 | 12 |
| 31.0 | 10 |
| 31.3 | 13 |
| 31.8 | 6 |
| 32.4 | 8 |
| 32.8 | 8 |
| 33.1 | 3 |
| 33.6 | 5 |
| 34.3 | 13 |
| 34.7 | 4 |
| 34.8 | 5 |
| 35.9 | 5 |
| 36.9 | 7 |
| 37.8 | 10 |
| 38.6 | 1 |

### Compound of Formula (I) Form I

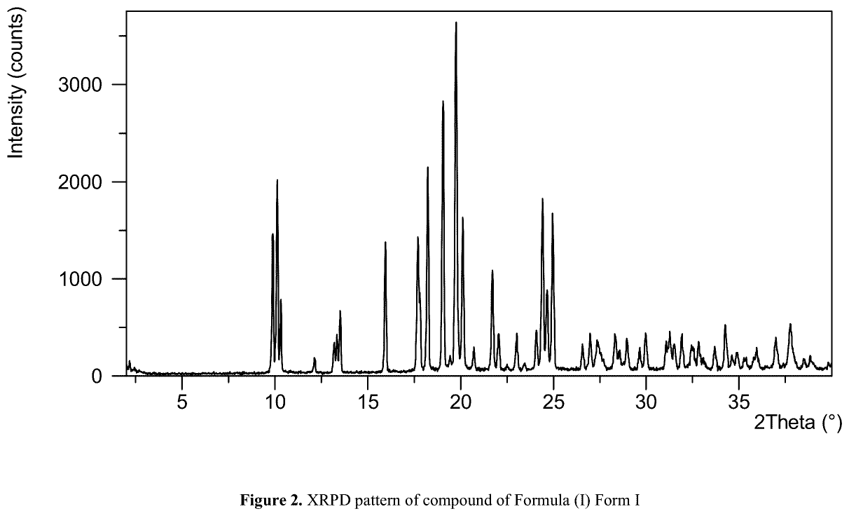

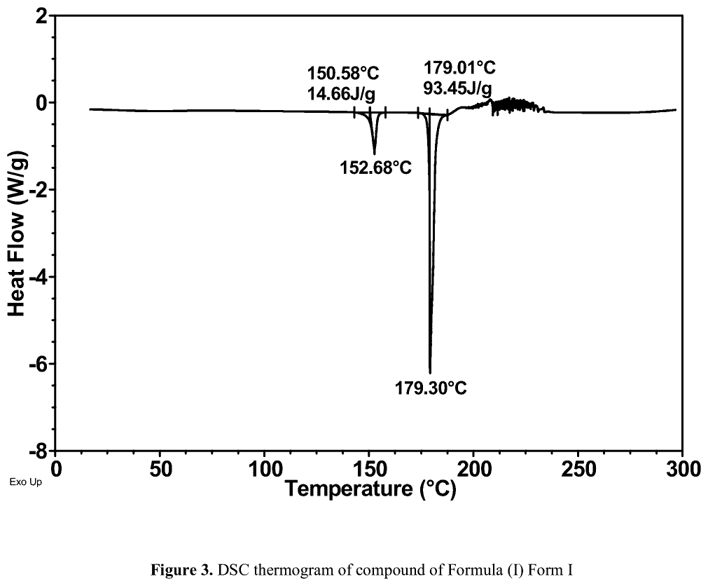

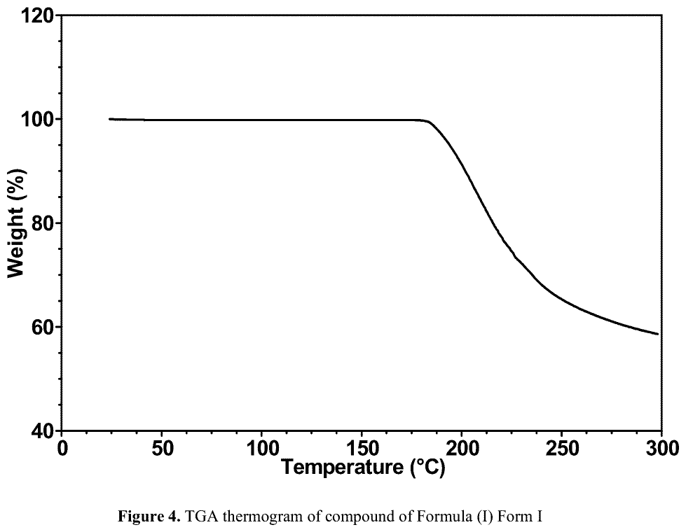

In some embodiments, the present disclosure provides a crystalline Form I of the compound of Formula (I) (compound of Formula (I) Form I). The compound of Formula (I) Form I exhibits an X-ray powder diffraction (XRPD) pattern substantially as shown in FIG. 2. The compound of Formula (I), Form I may exhibit a differential scanning calorimetry (DSC) thermogram substantially as shown in FIG. 3. The compound of Formula (I) Form I may exhibit a thermogravimetric analysis (TGA) graph substantially as shown in FIG. 4. The compound of Formula (I) Form I is dehydrated (i.e. free of water).

In some embodiments of the compound of Formula (I) Form I, at least one, at least two, at least three, or all of the following (a)-(c) apply: (a) compound of Formula (I) Form I has an XRPD pattern substantially as shown in FIG. 2; (b) compound of Formula (I) Form I has a DSC thermogram substantially as shown in FIG. 3; and (c) compound of Formula (I) Form I has a TGA graph substantially as shown in FIG. 4.

In some embodiments, compound of Formula (I) Form I has an XRPD pattern displaying at least two, at least three, at least four, at least five, or at least six of the 2θ-reflections as the XRPD pattern substantially as shown in FIG. 2.

In some embodiments, compound of Formula (I) Form I has an XRPD pattern comprising 2θ-reflections (±0.2 degrees 2θ) at 10.1°, 16.0°, and 19.8°. In some embodiments, compound of Formula (I) Form I has an XRPD pattern comprising 2θ-reflections (±0.2 degrees 2θ) at 10.1°, 16.0°, and 19.8°, and one, two or three of the 2θ-reflections (±0.2 degrees 2θ) at 9.9°, 13.5°, and 19.1°. In some embodiments, compound of Formula (I) Form I has an XRPD pattern comprising 2θ-reflections (±0.2 degrees 2θ) at 10.1°, 16.0°, and 19.8°, and one or two of the 2θ-reflections (±0.2 degrees 2θ) at 9.9°, 13.5°, and 19.1°. In some embodiments, compound of Formula (I) Form I has an XRPD pattern comprising 2θ-reflections (±0.2 degrees 2θ) at 16.0°, and 19.8°, and one of the 2θ-reflections (±0.2 degrees 2θ) at 9.9°, 13.5°, and 19.1°. In some embodiments, compound of Formula (I) Form I has an XRPD pattern comprising 2θ-reflections (±0.2 degrees 2θ) at 10.1°, 16.0°, and 19.8°, and two of the 2θ-reflections (±0.2 degrees 2θ) at 9.9°, 13.5°, and 19.1°. In some embodiments, compound of Formula (I) Form I has an XRPD pattern comprising 2θ-reflections (±0.2 degrees 2θ) at 9.9°, 10.1°, 13.5°, 16.0°, 19.1°, and 19.8°. In some embodiments, compound of Formula (I) Form I has an XRPD pattern comprising any three 2θ-reflections (±0.2 degrees 2θ) selected from the group consisting of 9.9°, 10.1°, 13.5°, 16.0°, 19.1°, and 19.8°.

In some embodiments, compound of Formula (I) Form I has an XRPD pattern comprising 2θ-reflections (±0.2 degrees 2θ) at 9.9°, 10.1°, 13.5°, 16.0°, 19.1°, and 19.8°, and one, two, or three of the 2θ-reflections (±0.2 degrees 2θ) at 18.2°, 20.1°, and 24.4°. In some embodiments, compound of Formula (I) Form I has an XRPD pattern comprising 2θ-reflections (±0.2 degrees 2θ) at 9.9°, 10.1°, 13.5°, 16.0°, 19.1°, and 19.8°, and one or two of the 2θ-reflections (±0.2 degrees 2θ) at 18.2°, 20.1°, and 24.4°. In some embodiments, compound of Formula (I) Form I has an XRPD pattern comprising 2θ-reflections (±0.2 degrees 2θ) at 9.9°, 13.5°, 16.0°, 19.1°, and 19.8°, and one of the 2θ-reflections (±0.2 degrees 2θ) at 18.2°, and 24.4°. In some embodiments, compound of Formula (I) Form I has an XRPD pattern comprising 2θ-reflections (±0.2 degrees 2θ) at 9.9°, 10.1°, 13.5°, 16.0°, 19.1°, and 19.8°, and two of the 2θ-reflections (±0.2 degrees 2θ) at 18.2°, 20.1°, and 24.4°. In some embodiments, compound of Formula (I) Form I has an XRPD pattern comprising 2θ-reflections (±0.2 degrees 2θ) at 9.9°, 10.1°, 13.5°, 16.0°, 18.2°, 19.1°, 19.8°, 20.1°, and 24.4°. In some embodiments, compound of Formula (I) Form I has an XRPD pattern comprising three of the 2θ-reflections (±0.2 degrees 2θ) at 9.9°, 10.1°, 13.5°, 16.0°, 18.2°, 19.1°, 19.8°, 20.1°, and 24.4°.

In some embodiments, compound of Formula (I) Form I has a XRPD pattern comprising peaks at:

| Pos. [°2θ] | Rel. Int. [%] |
|---|---|
| 2.2 | 3 |
| 9.9 | 40 |
| 10.1 | 54 |
| 10.3 | 20 |
| 12.1 | 4 |
| 13.2 | 8 |
| 13.3 | 10 |
| 13.5 | 17 |
| 16.0 | 37 |
| 17.7 | 38 |
| 17.8 | 20 |
| 18.2 | 58 |
| 19.1 | 76 |
| 19.8 | 100 |
| 20.1 | 43 |
| 20.7 | 6 |
| 21.7 | 29 |
| 22.1 | 10 |
| 22.5 | 1 |
| 23.0 | 9 |
| 23.5 | 2 |
| 24.1 | 11 |
| 24.4 | 48 |
| 24.7 | 23 |
| 25.0 | 44 |
| 26.6 | 7 |
| 27.0 | 10 |
| 27.4 | 8 |
| 28.3 | 10 |
| 28.6 | 5 |
| 29.0 | 9 |
| 29.6 | 6 |
| 30.0 | 10 |
| 31.1 | 7 |
| 31.3 | 10 |
| 31.5 | 6 |
| 31.9 | 9 |
| 32.4 | 6 |
| 32.8 | 8 |
| 33.7 | 6 |
| 34.3 | 12 |
| 34.6 | 3 |
| 34.9 | 4 |
| 35.3 | 2 |
| 36.0 | 5 |
| 37.0 | 8 |
| 37.8 | 13 |
| 38.5 | 2 |
| 38.8 | 3 |

In some embodiments, compound of Formula (I) Form I is characterized by a DSC thermogram substantially as shown in FIG. 3.

In some embodiments, compound of Formula (I) Form I is characterized by a DSC thermogram having one or both of (i) an endothermic transition at about 151° C. and (ii) an endotherm with onset at about 179° C.

In some embodiments, compound of Formula (I) Form I is characterized by a TGA curve substantially as shown in FIG. 4.

### Compound of Formula (I), Solvated Forms ACN1, ACN2, and ACN3

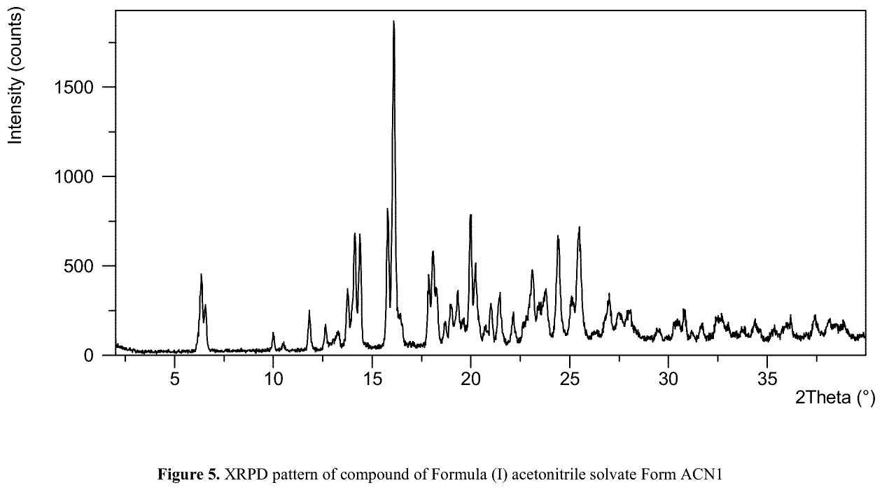

In some embodiments, the disclosure provides solvated crystalline forms of the compound of Formula (I). In some embodiments, the disclosure provides acetonitrile solvate forms of the compound of Formula (I). In some embodiments, the disclosure provides acetonitrile solvate form ACN1 of the compound of Formula (I) (“crystalline Form ACN1”). In some embodiments, crystalline Form ACN1 has an XRPD profile substantially as shown in FIG. 5. In some embodiments, crystalline Form ACN1 has an XRPD pattern displaying at least two, at least three, at least four, at least five, or at least six of the 2θ-reflections as the XRPD pattern substantially as shown in FIG. 5.

In some embodiments, the crystalline Form ACN1 is characterized by an XRPD pattern comprising 2θ-reflections (±0.2 degrees 2θ) at about 6.4°, 11.8°, and 16.1°. In some embodiments, the crystalline Form ACN1 has an XRPD pattern comprising 2θ-reflections (±0.2 degrees 2θ) at 6.4°, 11.8°, and 16.1°, and one, two or three of the 2θ-reflections (±0.2 degrees 2θ) at 10.0°, 14.1°, and 20.0°. In some embodiments, the crystalline Form ACN1 has an XRPD pattern comprising 2θ-reflections (±0.2 degrees 2θ) at 6.4°, 11.8°, and 16.1°, and one or two of the 2θ-reflections (±0.2 degrees 2θ) at 10.0°, 14.1°, and 20.0°. In some embodiments, the crystalline Form ACN1 has an XRPD pattern comprising 2θ-reflections (±0.2 degrees 2θ) at 6.4°, 11.8°, and 16.1°, and one of the 2θ-reflections (±0.2 degrees 2θ) at 10.0°, 14.1°, and 20.0°. In some embodiments, the crystalline Form ACN1 has an XRPD pattern comprising 2θ-reflections (±0.2 degrees 2θ) at 6.4°, 11.8°, and 16.1°, and two of the 2θ-reflections (±0.2 degrees 2θ) at 10.0°, 14.1°, and 20.0°. In some embodiments, the crystalline Form ACN1 has an XRPD pattern comprising 2θ-reflections (±0.2 degrees 2θ) at 6.4°, 10.0°, 11.8°, 14.1°, 16.1°, and 20.0°. In some embodiments, the crystalline Form ACN1 has an XRPD pattern comprising any three 2θ-reflections (±0.2 degrees 2θ) selected from the group consisting of 6.4°, 10.0°, 11.8°, 14.1°, 16.1°, and 20.0°.

In some embodiments, the crystalline Form ACN1 has an XRPD pattern comprising 2θ-reflections (±0.2 degrees 2θ) at 6.4°, 10.0°, 11.8°, 14.1°, 16.1°, and 20.0°, and one, two, or three of the 2θ-reflections (±0.2 degrees 2θ) at 15.8°, 18.1°, and 25.5°. In some embodiments, the crystalline Form ACN1 has an XRPD pattern comprising 2θ-reflections (±0.2 degrees 2θ) at 6.4°, 10.0°, 11.8°, 14.1°, 16.1°, and 20.0°, and one or two of the 2θ-reflections (±0.2 degrees 2θ) at 15.8°, 18.1°, and 25.5°. In some embodiments, the crystalline Form ACN1 has an XRPD pattern comprising 2θ-reflections (±0.2 degrees 2θ) at 6.4°, 10.0°, 11.8°, 14.1°, 16.1°, and and one of the 2θ-reflections (±0.2 degrees 2θ) at 15.8°, 18.1°, and 25.5°. In some embodiments, the crystalline Form ACN1 has an XRPD pattern comprising 2θ-reflections (±0.2 degrees 2θ) at 6.4°, 10.0°, 11.8°, 14.1°, 16.1°, and 20.0°, and two of the 2θ-reflections (±0.2 degrees 2θ) at 15.8°, 18.1°, and 25.5°. In some embodiments, the crystalline Form ACN1 has an XRPD pattern comprising 2θ-reflections (±0.2 degrees 2θ) at 6.4°, 10.0°, 11.8°, 14.1°, 15.8°, 16.1°, 18.1, 20.0°, and 25.5°. In some embodiments, the crystalline Form ACN1 has an XRPD pattern comprising three of the 2θ-reflections (±0.2 degrees 2θ) at 6.4°, 10.0°, 11.8°, 14.1°, 16.1°, 18.1, 20.0°, and 25.5°.

In some embodiments, the crystalline Form ACN1 has a XRPD pattern comprising peaks at:

| Pos. [°2θ] | Rel. Int. [%] |
|---|---|
| 6.4 | 23 |
| 6.6 | 13 |
| 10.0 | 6 |
| 10.5 | 2 |
| 11.8 | 12 |
| 12.6 | 6 |
| 13.3 | 4 |
| 13.8 | 18 |
| 14.1 | 34 |
| 14.4 | 32 |
| 15.8 | 41 |
| 16.1 | 100 |
| 16.5 | 8 |
| 17.9 | 19 |
| 18.1 | 27 |
| 18.3 | 15 |
| 18.7 | 5 |
| 19.0 | 10 |
| 19.3 | 13 |
| 20.0 | 37 |
| 20.2 | 22 |
| 21.0 | 8 |
| 21.5 | 13 |
| 22.1 | 9 |
| 23.1 | 21 |
| 23.8 | 13 |
| 24.5 | 26 |
| 25.1 | 10 |
| 25.5 | 32 |
| 27.0 | 11 |
| 28.1 | 7 |
| 29.5 | 3 |
| 30.4 | 4 |
| 30.8 | 9 |
| 31.7 | 4 |
| 32.4 | 5 |
| 33.8 | 2 |
| 34.4 | 6 |
| 35.3 | 3 |
| 36.2 | 5 |
| 37.4 | 6 |
| 38.9 | 4 |

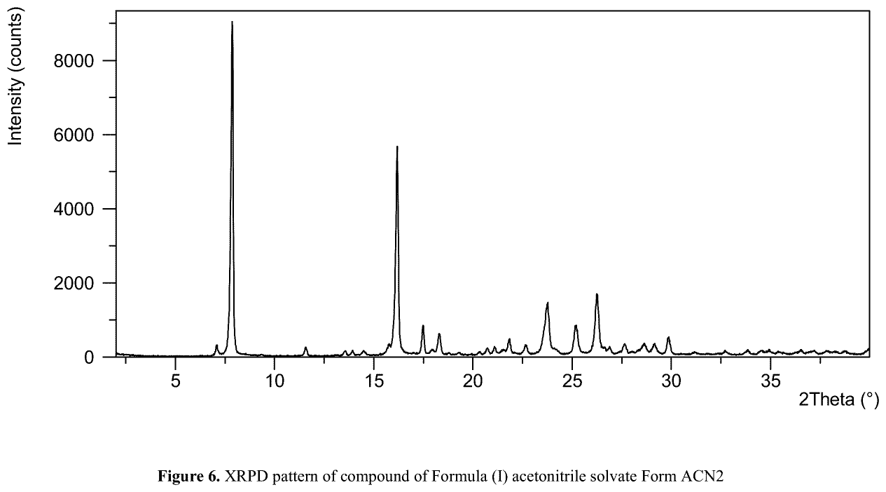

In some embodiments, the disclosure provides acetonitrile solvate form ACN2 of the compound of Formula (I) (“crystalline Form ACN2”). In some embodiments, crystalline Form ACN2 has an XRPD profile substantially as shown in FIG. 6. In some embodiments, crystalline Form ACN2 has an XRPD pattern displaying at least two, at least three, at least four, at least five, or at least six of the 2θ-reflections as the XRPD pattern substantially as shown in FIG. 6.

In some embodiments, the crystalline Form ACN2 is characterized by an XRPD pattern comprising 2θ-reflections (±0.2 degrees 2θ) at about 7.9°, 16.2°, and 23.8°. In some embodiments, the crystalline Form ACN2 has an XRPD pattern comprising 2θ-reflections (±0.2 degrees 2θ) at 7.9°, 16.2°, and 23.8°, and one, two or three of the 2θ-reflections (±0.2 degrees 2θ) at 7.1°, 11.6°, and 17.5°. In some embodiments, the crystalline Form ACN2 has an XRPD pattern comprising 2θ-reflections (±0.2 degrees 2θ) at 7.9°, 16.2°, and 23.8°, and one or two of the 2θ-reflections (±0.2 degrees 2θ) at 7.1°, 11.6°, and 17.5°. In some embodiments, the crystalline Form ACN2 has an XRPD pattern comprising 2θ-reflections (±0.2 degrees 2θ) at 7.9°, 16.2°, and 23.8°, and one of the 2θ-reflections (±0.2 degrees 2θ) at 7.1°, 11.6°, and 17.5°. In some embodiments, the crystalline Form ACN2 has an XRPD pattern comprising 2θ-reflections (±0.2 degrees 2θ) at 7.9°, 16.2°, and 23.8°, and two of the 2θ-reflections (±0.2 degrees 2θ) at 7.1°, 11.6°, and 17.5°. In some embodiments, the crystalline Form ACN2 has an XRPD pattern comprising 2θ-reflections (±0.2 degrees 2θ) at 7.1°, 7.9°, 11.6°, 16.2°, 17.5°, and 23.8°. In some embodiments, the crystalline Form ACN2 has an XRPD pattern comprising any three 2θ-reflections (±0.2 degrees 2θ) selected from the group consisting of 7.1°, 7.9°, 11.6°, 16.2°, 17.5°, and 23.8°.

In some embodiments, the crystalline Form ACN2 has an XRPD pattern comprising 2θ-reflections (±0.2 degrees 2θ) at 7.1°, 7.9°, 11.6°, 16.2°, 17.5°, and 23.8°, and one, two, or three of the 2θ-reflections (±0.2 degrees 2θ) at 18.3°, 25.2°, and 26.3°. In some embodiments, the crystalline Form ACN2 has an XRPD pattern comprising 2θ-reflections (±0.2 degrees 2θ) at 7.1°, 7.9°, 11.6°, 16.2°, 17.5°, and 23.8°, and one or two of the 2θ-reflections (±0.2 degrees 2θ) at 18.3°, 25.2°, and 26.3°. In some embodiments, the crystalline Form ACN2 has an XRPD pattern comprising 2θ-reflections (±0.2 degrees 2θ) at 7.1°, 7.9°, 11.6°, 16.2°, 17.5°, and 23.8°, and one of the 2θ-reflections (±0.2 degrees 2θ) at 18.3°, 25.2°, and 26.3°. In some embodiments, the crystalline Form ACN2 has an XRPD pattern comprising 2θ-reflections (±0.2 degrees 2θ) at 7.1°, 7.9°, 11.6°, 16.2°, 17.5°, and 23.8°, and two of the 2θ-reflections (±0.2 degrees 2θ) at 18.3°, 25.2°, and 26.3°. In some embodiments, the crystalline Form ACN2 has an XRPD pattern comprising 2θ-reflections (±0.2 degrees 2θ) at 7.1°, 7.9°, 11.6°, 16.2°, 17.5°, 18.3°, 23.8°, 25.2°, and 26.3°. In some embodiments, the crystalline Form ACN2 has an XRPD pattern comprising three of the 2θ-reflections (±0.2 degrees 2θ) at 7.1°, 7.9°, 11.6°, 16.2°, 17.5°, 18.3°, 23.8°, 25.2°, and 26.3°.

In some embodiments, the crystalline Form ACN2 has a XRPD pattern comprising peaks at:

| Pos. [°2θ] | Rel. Int. [%] |
|---|---|
| 7.1 | 3 |
| 7.9 | 100 |
| 11.6 | 2 |
| 13.6 | 1 |
| 13.9 | 1 |
| 14.5 | 1 |
| 15.7 | 3 |
| 16.2 | 61 |
| 17.5 | 8 |
| 17.9 | 1 |
| 18.3 | 6 |
| 18.8 | 0 |
| 19.3 | 1 |
| 20.3 | 1 |
| 20.7 | 2 |
| 21.1 | 2 |
| 21.8 | 4 |
| 22.7 | 3 |
| 23.8 | 15 |
| 25.2 | 8 |
| 26.3 | 18 |
| 26.9 | 2 |
| 27.7 | 2 |
| 28.6 | 2 |
| 29.1 | 3 |
| 29.9 | 5 |
| 31.2 | 1 |
| 32.7 | 1 |
| 33.9 | 1 |
| 34.5 | 1 |
| 34.9 | 1 |
| 35.4 | 0 |
| 36.6 | 1 |
| 37.2 | 1 |
| 37.8 | 1 |
| 38.8 | 1 |

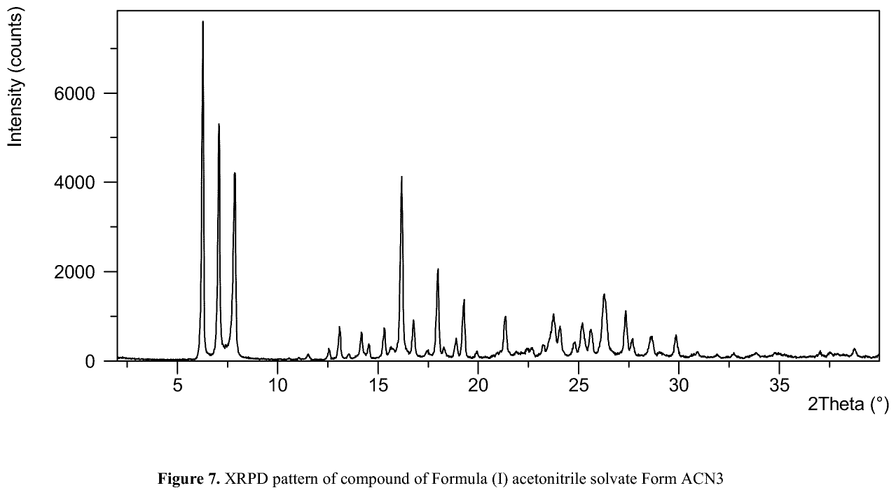

In some embodiments, the disclosure provides acetonitrile solvate form ACN3 of the compound of Formula (I) (“crystalline Form ACN3”). In some embodiments, crystalline Form ACN3 has an XRPD profile substantially as shown in FIG. 7. In some embodiments, crystalline Form ACN3 has an XRPD pattern displaying at least two, at least three, at least four, at least five, or at least six of the 2θ-reflections as the XRPD pattern substantially as shown in FIG. 7.

In some embodiments, the crystalline Form ACN3 is characterized by an XRPD pattern comprising 2θ-reflections (±0.2 degrees 2θ) at about 6.3°, 7.9°, and 16.2°. In some embodiments, the crystalline Form ACN3 has an XRPD pattern comprising 2θ-reflections (±0.2 degrees 2θ) at 6.3°, 7.9°, and 16.2°, and one, two or three of the 2θ-reflections (±0.2 degrees 2θ) at 7.1°, 13.1°, and 18.0°. In some embodiments, the crystalline Form ACN3 has an XRPD pattern comprising 2θ-reflections (±0.2 degrees 2θ) at 6.3°, 7.9°, and 16.2°, and one or two of the 2θ-reflections (±0.2 degrees 2θ) at 7.1°, 13.1°, and 18.0°. In some embodiments, the crystalline Form ACN3 has an XRPD pattern comprising 2θ-reflections (±0.2 degrees 2θ) at 6.3°, 7.9°, and 16.2°, and one of the 2θ-reflections (±0.2 degrees 2θ) at 7.1°, 13.1°, and 18.0°. In some embodiments, the crystalline Form ACN3 has an XRPD pattern comprising 2θ-reflections (±0.2 degrees 2θ) at 6.3°, 7.9°, and 16.2°, and two of the 2θ-reflections (±0.2 degrees 2θ) at 7.1°, 13.1°, and 18.0°. In some embodiments, the crystalline Form ACN3 has an XRPD pattern comprising 2θ-reflections (±0.2 degrees 2θ) at 6.3°, 7.1°, 7.9°, 13.1°, 16.2°, and 18.0°. In some embodiments, the crystalline Form ACN3 has an XRPD pattern comprising any three 2θ-reflections (±0.2 degrees 2θ) selected from the group consisting of 6.3°, 7.1°, 7.9°, 13.1°, 16.2°, and 18.0°.

In some embodiments, the crystalline Form ACN3 has an XRPD pattern comprising 2θ-reflections (±0.2 degrees 2θ) at 6.3°, 7.1°, 7.9°, 13.1°, 16.2°, and 18.0°, and one, two, or three of the 2θ-reflections (±0.2 degrees 2θ) at 15.3°, 19.3°, and 21.3°. In some embodiments, the crystalline Form ACN3 has an XRPD pattern comprising 2θ-reflections (±0.2 degrees 2θ) at 6.3°, 7.1°, 7.9°, 13.1°, 16.2°, and 18.0°, and one or two of the 2θ-reflections (±0.2 degrees 2θ) at 15.3°, 19.3°, and 21.3°. In some embodiments, the crystalline Form ACN3 has an XRPD pattern comprising 2θ-reflections (±0.2 degrees 2θ) at 6.3°, 7.1°, 7.9°, 13.1°, 16.2°, and 18.0°, and one of the 2θ-reflections (±0.2 degrees 2θ) at 15.3°, 19.3°, and 21.3°. In some embodiments, the crystalline Form ACN3 has an XRPD pattern comprising 2θ-reflections (±0.2 degrees 2θ) at 6.3°, 7.1°, 7.9°, 13.1°, 16.2°, and 18.0°, and two of the 2θ-reflections (±0.2 degrees 2θ) at 15.3°, 19.3°, and 21.3°. In some embodiments, the crystalline Form ACN3 has an XRPD pattern comprising 2θ-reflections (±0.2 degrees 2θ) at 6.3°, 7.1°, 7.9°, 13.1°, 15.3°, 16.2°, 18.0°, 19.3°, and 21.3°. In some embodiments, the crystalline Form ACN3 has an XRPD pattern comprising three of the 2θ-reflections (±0.2 degrees 2θ) at 6.3°, 7.1°, 7.9°, 13.1°, 15.3°, 16.2°, 18.0°, 19.3°, and 21.3°.

In some embodiments, the crystalline Form ACN3 has a XRPD pattern comprising peaks at:

| Pos. [°2θ] | Rel. Int. [%] |
|---|---|
| 6.3 | 100 |
| 7.1 | 70 |
| 7.9 | 56 |
| 10.5 | 0 |
| 11.5 | 2 |
| 12.6 | 3 |
| 13.1 | 9 |
| 13.6 | 1 |
| 14.2 | 8 |
| 14.6 | 4 |
| 15.3 | 8 |
| 15.6 | 2 |
| 16.2 | 52 |
| 16.8 | 10 |
| 17.5 | 1 |
| 18.0 | 26 |
| 18.3 | 2 |
| 18.9 | 5 |
| 19.3 | 17 |
| 19.9 | 2 |
| 21.3 | 11 |
| 22.4 | 2 |
| 22.7 | 2 |
| 23.2 | 3 |
| 23.8 | 2 |
| 24.1 | 8 |
| 24.8 | 3 |
| 25.2 | 9 |
| 25.6 | 6 |
| 26.3 | 17 |
| 27.3 | 13 |
| 27.7 | 4 |
| 28.6 | 5 |
| 29.9 | 6 |
| 30.9 | 2 |
| 31.9 | 1 |
| 32.7 | 1 |
| 33.8 | 1 |
| 34.8 | 1 |
| 37.0 | 1 |
| 37.5 | 1 |
| 38.8 | 2 |

### Compound of Formula (I) Form II

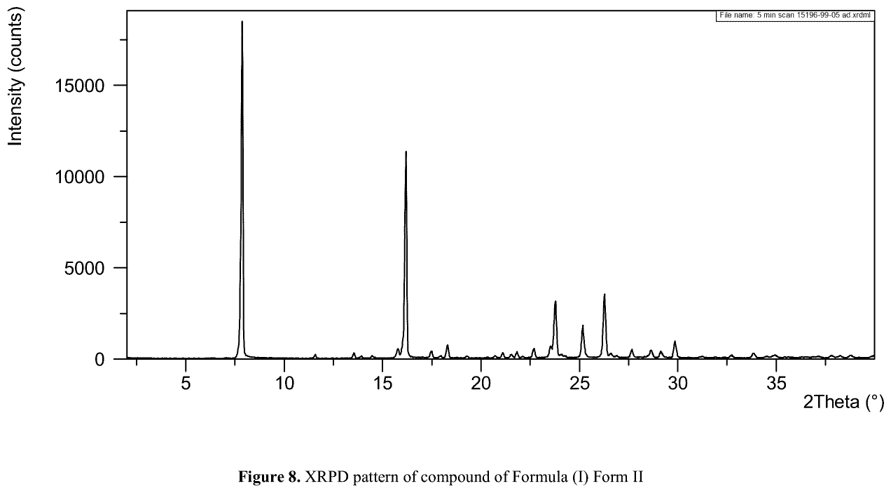

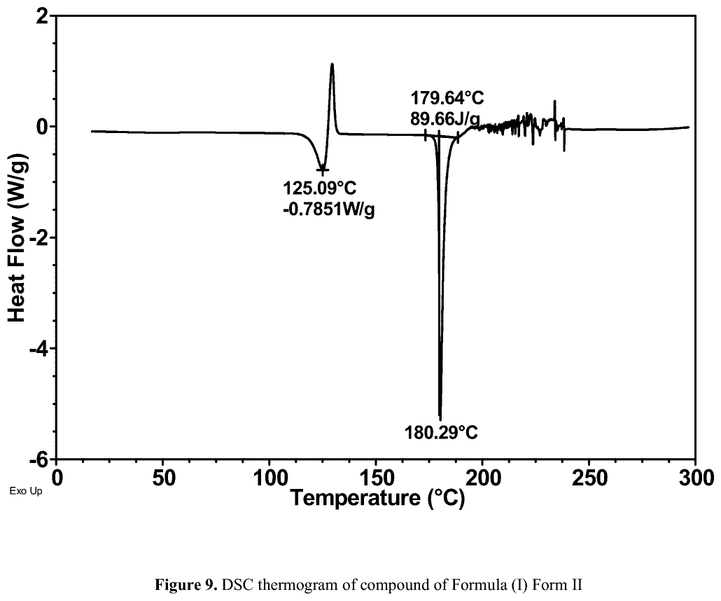

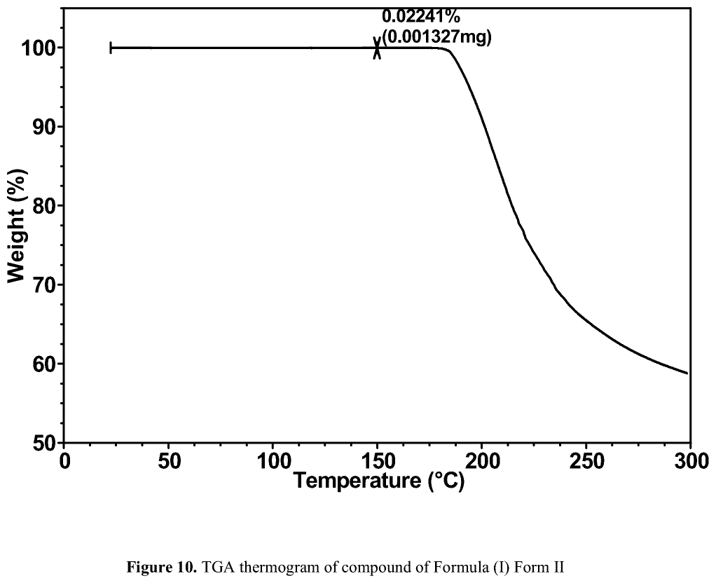

In some embodiments, the present disclosure provides a crystalline Form II of the compound of Formula (I) (compound of Formula (I) Form II). The compound of Formula (I) Form II exhibits an X-ray powder diffraction (XRPD) pattern substantially as shown in FIG. 8. The compound of Formula (I), Form II may exhibit a differential scanning calorimetry (DSC) thermogram substantially as shown in FIG. 9. The compound of Formula (I) Form II may exhibit a thermogravimetric analysis (TGA) graph substantially as shown in FIG. 10. The compound of Formula (I) Form II is desolvated (i.e. free of solvent).

In some embodiments of the compound of Formula (I) Form II, at least one, at least two, at least three, or all of the following (a)-(c) apply: (a) compound of Formula (I) Form II has an XRPD pattern substantially as shown in FIG. 8; (b) compound of Formula (I) Form II has a DSC thermogram substantially as shown in FIG. 9; and (c) compound of Formula (I) Form II has a TGA graph substantially as shown in FIG. 10.

In some embodiments, compound of Formula (I) Form II has an XRPD pattern displaying at least two, at least three, at least four, at least five, or at least six of the 2θ-reflections as the XRPD pattern substantially as shown in FIG. 8.

In some embodiments, compound of Formula (I) Form II has an XRPD pattern comprising 2θ-reflections (±0.2 degrees 2θ) at 7.9°, 16.2°, and 23.8°. In some embodiments, compound of Formula (I) Form II has an XRPD pattern comprising 2θ-reflections (±0.2 degrees 2θ) at 7.9°, 16.2°, and 23.8°, and one, two or three of the 2θ-reflections (±0.2 degrees 2θ) at 13.6°, 18.3°, and 26.3°. In some embodiments, compound of Formula (I) Form II has an XRPD pattern comprising 2θ-reflections (±0.2 degrees 2θ) at 7.9°, 16.2°, and 23.8°, and one or two of the 2θ-reflections (±0.2 degrees 2θ) at 13.6°, 18.3°, and 26.3°. In some embodiments, compound of Formula (I) Form II has an XRPD pattern comprising 2θ-reflections (±0.2 degrees 2θ) at 7.9°, 16.2°, and 23.8°, and one of the 2θ-reflections (±0.2 degrees 2θ) at 13.6°, 18.3°, and 26.3°. In some embodiments, compound of Formula (I) Form II has an XRPD pattern comprising 2θ-reflections (±0.2 degrees 2θ) at 7.9°, 16.2°, and 23.8°, and two of the 2θ-reflections (±0.2 degrees 2θ) at 13.6°, 18.3°, and 26.3°. In some embodiments, compound of Formula (I) Form II has an XRPD pattern comprising 2θ-reflections (±0.2 degrees 2θ) at 7.9°, 13.6°, 16.2°, 18.3°, 23.8°, and 26.3°. In some embodiments, compound of Formula (I) Form II has an XRPD pattern comprising any three 2θ-reflections (±0.2 degrees 2θ) selected from the group consisting of 7.9°, 13.6°, 16.2°, 18.3°, 23.8°, and 26.3°.

In some embodiments, compound of Formula (I) Form II has an XRPD pattern comprising 2θ-reflections (±0.2 degrees 2θ) at 7.9°, 13.6°, 16.2°, 18.3°, 23.8°, and 26.3°, and one, two, or three of the 2θ-reflections (±0.2 degrees 2θ) at 17.5°, 25.2°, and 29.9°. In some embodiments, compound of Formula (I) Form II has an XRPD pattern comprising 2θ-reflections (±0.2 degrees 2θ) at 7.9°, 13.6°, 16.2°, 18.3°, 23.8°, and 26.3°, and one or two of the 2θ-reflections (±0.2 degrees 2θ) at 17.5°, 25.2°, and 29.9°. In some embodiments, compound of Formula (I) Form II has an XRPD pattern comprising 2θ-reflections (±0.2 degrees 2θ) at 7.9°, 13.6°, 16.2°, 18.3°, 23.8°, and 26.3°, and one of the 2θ-reflections (±0.2 degrees 2θ) at 17.5°, and 29.9°. In some embodiments, compound of Formula (I) Form II has an XRPD pattern comprising 2θ-reflections (±0.2 degrees 2θ) at 7.9°, 13.6°, 16.2°, 18.3°, 23.8°, and 26.3°, and two of the 2θ-reflections (±0.2 degrees 2θ) at 17.5°, 25.2°, and 29.9°. In some embodiments, compound of Formula (I) Form II has an XRPD pattern comprising 2θ-reflections (±0.2 degrees 2θ) at 7.9°, 13.6°, 16.2°, 17.5°, 18.3°, 23.8°, 25.2°, 26.3°, and 29.9°. In some embodiments, compound of Formula (I) Form II has an XRPD pattern comprising three of the 2θ-reflections (±0.2 degrees 2θ) at 7.9°, 13.6°, 16.2°, 17.5°, 18.3°, 23.8°, 25.2°, 26.3°, and 29.9°.

In some embodiments, compound of Formula (I) Form II has a XRPD pattern comprising peaks at:

| Pos. [°2θ] | Rel. Int. [%] |
|---|---|
| 7.1 | 0 |
| 7.9 | 100 |
| 11.6 | 1 |
| 13.6 | 1 |
| 13.9 | 1 |
| 14.5 | 1 |
| 15.8 | 3 |
| 16.2 | 60 |
| 17.5 | 2 |
| 18.0 | 1 |
| 18.3 | 4 |
| 19.3 | 1 |
| 20.3 | 0 |
| 20.7 | 1 |
| 21.1 | 1 |
| 21.6 | 1 |
| 21.8 | 2 |
| 22.1 | 0 |
| 22.7 | 3 |
| 23.5 | 3 |
| 23.8 | 17 |
| 25.2 | 9 |
| 26.3 | 18 |
| 26.6 | 1 |
| 27.7 | 2 |
| 28.6 | 2 |
| 29.1 | 2 |
| 29.9 | 5 |
| 31.3 | 0 |
| 31.9 | 0 |
| 32.7 | 1 |
| 33.8 | 1 |
| 34.5 | 0 |
| 34.9 | 1 |
| 35.5 | 0 |
| 37.2 | 0 |
| 37.8 | 1 |
| 38.2 | 1 |
| 38.8 | 1 |

In some embodiments, compound of Formula (I) Form II is characterized by a DSC thermogram substantially as shown in FIG. 9.

In some embodiments, compound of Formula (I) Form II is characterized by a DSC thermogram having one or both of (i) endothermic transition at about 125° C. followed by an immediate exotherm, indicating a melting/recrystallization transition and (ii) second endotherm with onset at about 180° C.

In some embodiments, compound of Formula (I) Form II is characterized by a TGA curve substantially as shown in FIG. 10.

### Compound of Formula (I) Form III

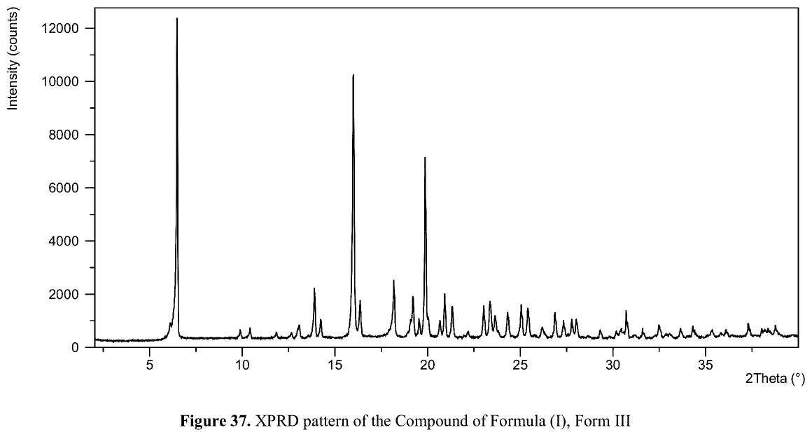

In some embodiments, the present disclosure provides a crystalline Form III of the compound of Formula (I) (compound of Formula (I) Form III). The compound of Formula (I) Form III exhibits an X-ray powder diffraction (XRPD) pattern substantially as shown in FIG. 37.

In some embodiments, compound of Formula (I) Form III has an XRPD pattern displaying at least two, at least three, at least four, at least five, or at least six of the 2θ-reflections as the XRPD pattern substantially as shown in FIG. 37.

In some embodiments, compound of Formula (I) Form III has an XRPD pattern comprising 2θ-reflections (±0.2 degrees 2θ) at 6.5°, 16.0°, and 19.9°. In some embodiments, compound of Formula (I) Form III has an XRPD pattern comprising 2θ-reflections (±0.2 degrees 2θ) at 6.5°, 16.0°, and 19.9°, and one, two or three of the 2θ-reflections (±0.2 degrees 2θ) at 13.9°, 18.2°, and 23.3°. In some embodiments, compound of Formula (I) Form III has an XRPD pattern comprising 2θ-reflections (±0.2 degrees 2θ) at 6.5°, 16.0°, and 19.9°, and one or two of the 2θ-reflections (±0.2 degrees 2θ) at 13.9°, 18.2°, and 23.3°. In some embodiments, compound of Formula (I) Form III has an XRPD pattern comprising 2θ-reflections (±0.2 degrees 2θ) at 6.5°, 16.0°, and 19.9°, and one of the 2θ-reflections (±0.2 degrees 2θ) at 13.9°, 18.2°, and 23.3°. In some embodiments, compound of Formula (I) Form III has an XRPD pattern comprising 2θ-reflections (±0.2 degrees 2θ) at 6.5°, 16.0°, and 19.9°, and two of the 2θ-reflections (±0.2 degrees 2θ) at 13.9°, 18.2°, and 23.3°. In some embodiments, compound of Formula (I) Form II has an XRPD pattern comprising 2θ-reflections (±0.2 degrees 2θ) at 6.5°, 13.9°, 16.0°, 18.2°, 19.9°, and 23.3°. In some embodiments, compound of Formula (I) Form III has an XRPD pattern comprising any three 2θ-reflections (±0.2 degrees 2θ) selected from the group consisting of 6.5°, 13.9°, 16.0°, 18.2°, 19.9°, and 23.3°.

In some embodiments, compound of Formula (I) Form III has an XRPD pattern comprising 2θ-reflections (±0.2 degrees 2θ) at 6.5°, 13.9°, 16.0°, 18.2°, 19.9°, and 23.3°, and one, two, or three of the 2θ-reflections (±0.2 degrees 2θ) at 10.4°, 19.2°, and 25.4°. In some embodiments, compound of Formula (I) Form III has an XRPD pattern comprising 2θ-reflections (±0.2 degrees 2θ) at 6.5°, 13.9°, 16.0°, 18.2°, 19.9°, and 23.3°, and one or two of the (±0.2 degrees 2θ) at 10.4°, 19.2°, and 25.4°. In some embodiments, compound of Formula (I) Form III has an XRPD pattern comprising 2θ-reflections (±0.2 degrees 2θ) at 6.5°, 13.9°, 16.0°, 18.2°, 19.9°, and 23.3°, and one of the 2θ-reflections (±0.2 degrees 2θ) at 10.4°, 19.2°, and 25.4°. In some embodiments, compound of Formula (I) Form III has an XRPD pattern comprising 2θ-reflections (±0.2 degrees 2θ) at 6.5°, 13.9°, 16.0°, 18.2°, 19.9°, and 23.3°, and two of the 2θ-reflections (±0.2 degrees 2θ) at 10.4°, 19.2°, and 25.4°. In some embodiments, compound of Formula (I) Form III has an XRPD pattern comprising 2θ-reflections (±0.2 degrees 2θ) at 6.5°, 10.4°, 13.9°, 16.0°, 18.2°, 19.2°, 19.9°, 23.3°, and 25.4°. In some embodiments, compound of Formula (I) Form III has an XRPD pattern comprising three of the 2θ-reflections (±0.2 degrees 2θ) at 6.5°, 10.4°, 13.9°, 16.0°, 18.2°, 19.2°, 19.9°, 23.3°, and 25.4°.

In some embodiments, compound of Formula (I) Form III has a XRPD pattern comprising peaks as shown in Table 36.

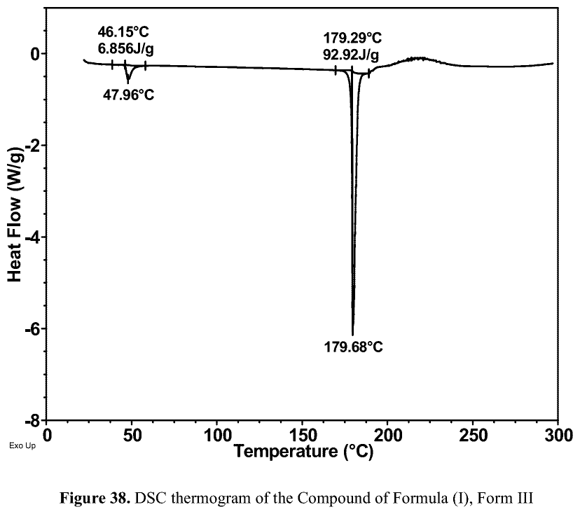

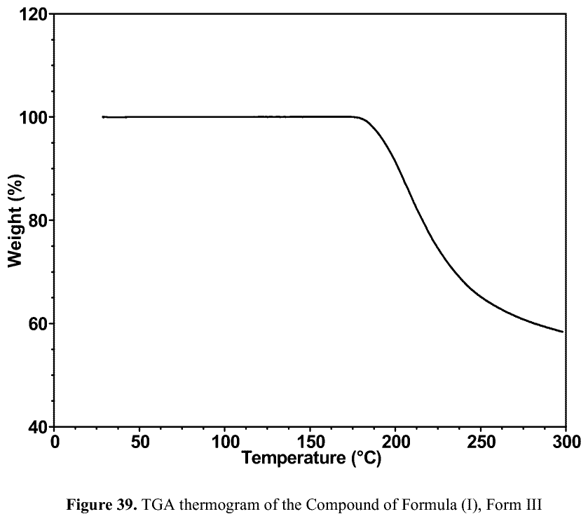

In some embodiments, compound of Formula (I) Form III is characterized by a DSC thermogram substantially as shown in FIG. 38. It has two endothermic events with onsets at 46° C. and 179° C. In some embodiments, compound of Formula (I) Form III is characterized by a TGA thermogram substantially as shown in FIG. 39. In some embodiments, compound of Formula (I) Form III is unsolvated.

### Compound of Formula (I) Form IV

In some embodiments, the present disclosure provides crystalline Form IV of the compound of Formula (I) (compound of Formula (I), Form IV):

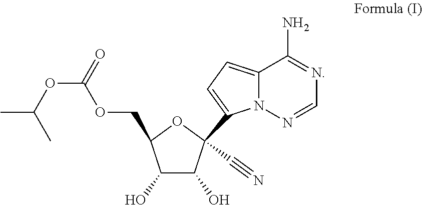 [SMILES: CC(C)OC(=O)OC[C@H]1O[C@@](C#N)(c2ccc3c(N)ncnn23)[C@H](O)[C@@H]1O]

In some embodiments, the compound of Formula (I), Form IV is characterized by an XRPD pattern comprising 2θ-reflections (±0.2 degrees 2θ) at about 6.7°, 10.2°, and 18.0°. In some embodiments, the compound of Formula (I), Form IV is characterized by an XRPD pattern comprising 2θ-reflections (±0.2 degrees 2θ) at about 6.7°, 10.2°, and 18.0°, and one, two, or three of the 2θ-reflections (±0.2 degrees 2θ) at about 9.0°, 16.5°, and 18.6°. In some embodiments, the compound of Formula (I), Form IV is characterized by an XRPD pattern comprising 2θ-reflections (±0.2 degrees 2θ) at about 6.7°, 10.2°, and 18.0°, and one or two of the 2θ-reflections (±0.2 degrees 2θ) at about 9.0°, 16.5°, and 18.6°. In some embodiments, the compound of Formula (I), Form IV is characterized by an XRPD pattern comprising 2θ-reflections (±0.2 degrees 2θ) at about 6.7°, 10.2°, and 18.0°, and one of the 2θ-reflections (±0.2 degrees 2θ) at about 9.0°, 16.5°, and 18.6°. In some embodiments, the compound of Formula (I), Form IV is characterized by an XRPD pattern comprising 2θ-reflections (±0.2 degrees 2θ) at about 6.7°, 10.2°, and 18.0°, and two of the 2θ-reflections (±0.2 degrees 2θ) at about 9.0°, 16.5°, and 18.6°. In some embodiments, the compound of Formula (I), Form IV is characterized by an XRPD pattern comprising 2θ-reflections (±0.2 degrees 2θ) at about 6.7°, 9.0°, 10.2°, 16.5°, 18.0°, and 18.6°.

In some embodiments, the compound of Formula (I), Form IV is characterized by an XRPD pattern comprising 2θ-reflections (±0.2 degrees 2θ) at about 6.7°, 9.0°, 10.2°, 16.5°, 18.0°, and 18.6°, and one, two, or three of the 2θ-reflections (±0.2 degrees 2θ) at about 10.9, and 25.6°. In some embodiments, the compound of Formula (I), Form IV is characterized by an XRPD pattern comprising 2θ-reflections (±0.2 degrees 2θ) at about 6.7°, 9.0°, 10.2°, 16.5°, 18.0°, and 18.6°, and one or two of the 2θ-reflections (±0.2 degrees 2θ) at about 10.9, and 25.6°. In some embodiments, the compound of Formula (I), Form IV is characterized by an XRPD pattern comprising 2θ-reflections (±0.2 degrees 2θ) at about 6.7°, 9.0°, 10.2°, 16.5°, 18.0°, and 18.6°, and one of the 2θ-reflections (±0.2 degrees 2θ) at about 10.9, 20.1, and 25.6°. In some embodiments, the compound of Formula (I), Form IV is characterized by an XRPD pattern comprising 2θ-reflections (±0.2 degrees 2θ) at about 6.7°, 9.0°, 10.2°, 16.5°, 18.0°, and 18.6°, and two of the 2θ-reflections (±0.2 degrees 2θ) at about 10.9, 20.1, and 25.6°. In some embodiments, the compound of Formula (I), Form IV is characterized by an XRPD pattern comprising 2θ-reflections (±0.2 degrees 2θ) at about 6.7°, 9.0°, 10.2°, 10.9, 16.5°, 18.0°, 18.6°, 20.1, and 25.6°.

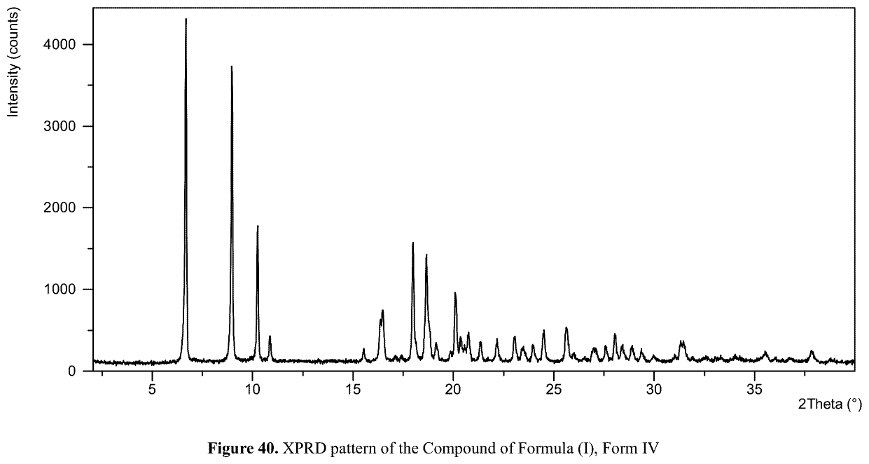

In some embodiments, the compound of Formula (I), Form IV is characterized by an XRPD pattern comprising peaks as listed in Table 38. In some embodiments, the compound of Formula (I), Form IV is characterized by an XRPD pattern substantially as shown in FIG. 40.

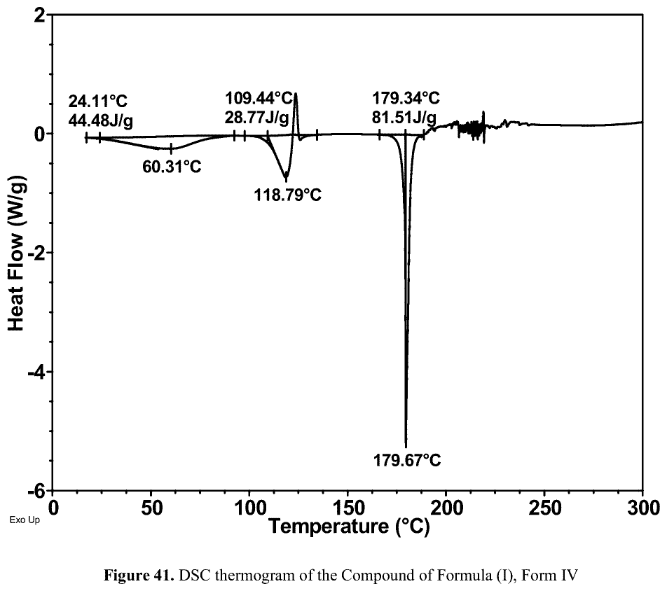

In some embodiments, the compound of Formula (I), Form IV is characterized by a DSC pattern comprising an endothermic event with an onset at about 25° C. In some embodiments, the compound of Formula (I), Form IV is characterized by a DSC pattern comprising an endothermic event with an onset at about 25° C. and a second endothermic transition at about 119° C. In some embodiments, the compound of Formula (I), Form IV is characterized by a DSC pattern comprising an endothermic event with an onset at about 25° C. and a second endothermic transition at about 119° C. followed by an immediate exotherm, indicating a melting/recrystallization transition. In some embodiments, the compound of Formula (I), Form IV is characterized by a DSC pattern comprising an endothermic event with an onset at about 25° C. and a second endothermic transition at about 119° C. and a third endothermic transition with an onset at about 179° C. In some embodiments, the compound of Formula (I), Form IV is characterized by a DSC pattern substantially as set forth in FIG. 41.

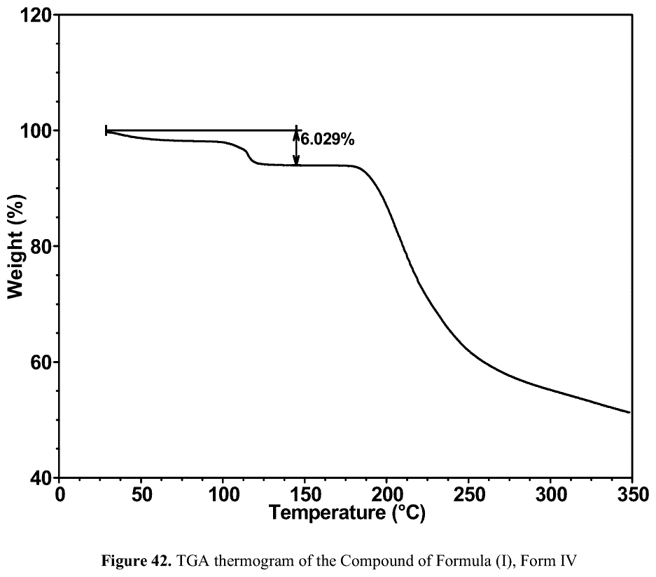

In some embodiments, the compound of Formula (I), Form IV is characterized by a TGA thermogram as shown in FIG. 42. In some embodiments, the compound of Formula (I), Form IV is solvated.

Salts of the compound of Formula (I)

In some embodiments, the present disclosure provides salts of the compound of Formula (I):

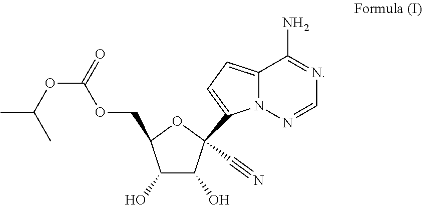 [SMILES: CC(C)OC(=O)OC[C@H]1O[C@@](C#N)(c2ccc3c(N)ncnn23)[C@H](O)[C@@H]1O]

In some embodiments, the present disclosure provides a HCl salt, an oxalate salt, a maleate salt, or a mesylate salt of the compound of Formula (I). In some embodiments, the salt is a HCl salt of the compound of Formula (I). In some embodiments, the salt is an oxalate salt of the compound of Formula (I). In some embodiments, the salt is a maleate salt of the compound of Formula (I). In some embodiments, the salt is a mesylate salt of the compound of Formula (I).

In some embodiments, the present disclosure provides a crystalline form of a salt of a compound of Formula (I):

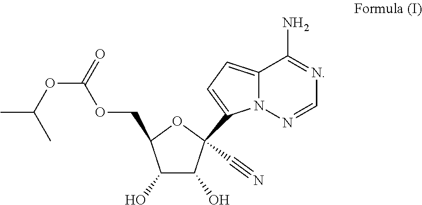 [SMILES: CC(C)OC(=O)OC[C@H]1O[C@@](C#N)(c2ccc3c(N)ncnn23)[C@H](O)[C@@H]1O]

In some embodiments, the disclosure provides a crystalline form of the HCl salt of the compound of Formula I.

### Compound of Formula (I), HCl Salt, Form I

In some embodiments, the disclosure provides a crystalline Form I of the HCl salt of the compound of Formula (I) (“compound of Formula (I), HCl salt, Form I”). In some embodiments, the compound of Formula (I), HCl salt, Form I is characterized by an XRPD pattern comprising 2θ-reflections (±0.2 degrees 2θ) at about 5.8°, 11.5°, and 18.0°. In some embodiments, the compound of Formula (I), HCl salt, Form I is characterized by an XRPD pattern comprising 2θ-reflections (±0.2 degrees 2θ) at about 5.8°, 11.5°, and 18.0°, and one, two, or three of the 2θ-reflections (±0.2 degrees 2θ) at about 14.1°, 20.0°, and 27.0°. In some embodiments, the compound of Formula (I), HCl salt, Form I is characterized by an XRPD pattern comprising 2θ-reflections (±0.2 degrees 2θ) at about 5.8°, 11.5°, and 18.0°, and one or two of the 2θ-reflections (±0.2 degrees 2θ) at about 14.1°, 20.0°, and 27.0°. In some embodiments, the compound of Formula (I), HCl salt, Form I is characterized by an XRPD pattern comprising 2θ-reflections (±0.2 degrees 2θ) at about 5.8°, 11.5°, and 18.0°, and one of the 2θ-reflections (±0.2 degrees 2θ) at about 14.1°, 20.0°, and 27.0°. In some embodiments, the compound of Formula (I), HCl salt, Form I is characterized by an XRPD pattern comprising 2θ-reflections (±0.2 degrees 2θ) at about 5.8°, 11.5°, and 18.0°, and two of the 2θ-reflections (±0.2 degrees 2θ) at about 14.1°, 20.0°, and 27.0°. In some embodiments, the compound of Formula (I), HCl salt, Form I is characterized by an XRPD pattern comprising 2θ-reflections (±0.2 degrees 2θ) at about 5.8°, 11.5°, 14.1°, 18.0°, 20.0°, and 27.0°.

In some embodiments, the compound of Formula (I), HCl salt, Form I is characterized by an XRPD pattern comprising 2θ-reflections (±0.2 degrees 2θ) at about 5.8°, 11.5°, 14.1°, 18.0°, 20.0°, and 27.0°, and one, two, or three of the 2θ-reflections (±0.2 degrees 2θ) at about 13.8°, 22.1°, and 29.5°. In some embodiments, the compound of Formula (I), HCl salt, Form I is characterized by an XRPD pattern comprising 2θ-reflections (±0.2 degrees 2θ) at about 5.8°, 11.5°, 14.1°, 18.0°, 20.0°, and 27.0°, and one or two of the 2θ-reflections (±0.2 degrees 2θ) at about 13.8°, 22.1°, and 29.5°. In some embodiments, the compound of Formula (I), HCl salt, Form I is characterized by an XRPD pattern comprising 2θ-reflections (±0.2 degrees 2θ) at about 5.8°, 11.5°, 14.1°, 18.0°, 20.0°, and 27.0°, and one of the 2θ-reflections (±0.2 degrees 2θ) at about 13.8°, 22.1°, and 29.5°. In some embodiments, the compound of Formula (I), HCl salt, Form I is characterized by an XRPD pattern comprising 2θ-reflections (±0.2 degrees 2θ) at about 5.8°, 11.5°, 14.1°, 18.0°, 20.0°, and 27.0°, and two of the 2θ-reflections (±0.2 degrees 2θ) at about 13.8°, 22.1°, and 29.5°. In some embodiments, the compound of Formula (I), HCl salt, Form I is characterized by an XRPD pattern comprising 2θ-reflections (±0.2 degrees 2θ) at about 5.8°, 11.5°, 13.8°, 14.1°, 18.0°, 20.0°, 22.1°, 27.0° and 29.5°.

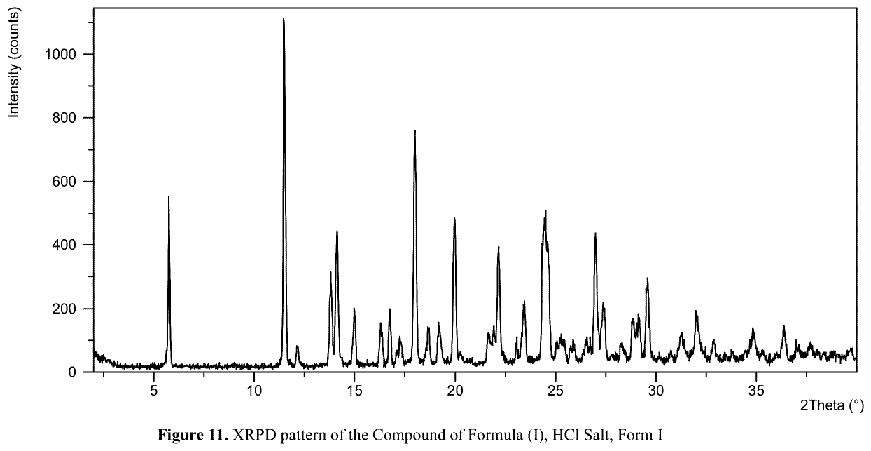

In some embodiments, the compound of Formula (I), HCl salt, Form I is characterized by an XRPD pattern comprising peaks as listed in Table 14. In some embodiments, the compound of Formula (I), HCl salt, Form I is characterized by an XRPD pattern substantially as shown in FIG. 11.

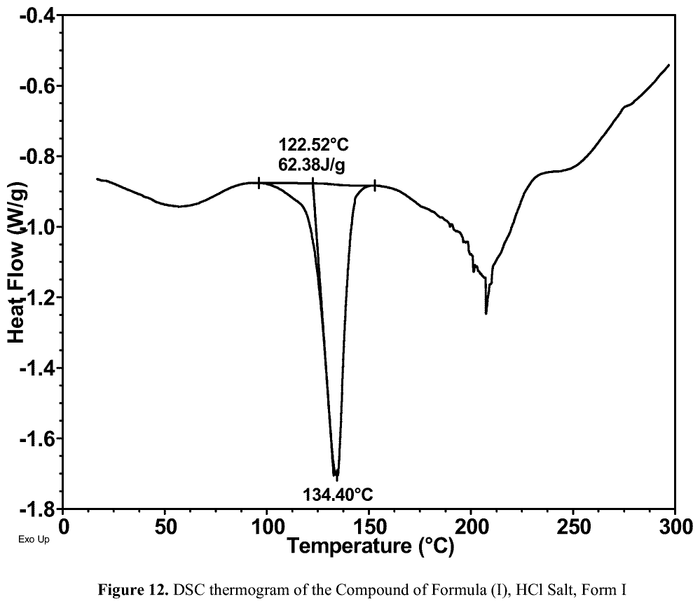

In some embodiments, the compound of Formula (I), HCl salt, Form I is characterized by a DSC pattern comprising an endothermic transition at about 123° C. In some embodiments, the compound of Formula (I), HCl salt, Form I is characterized by a DSC pattern substantially as set forth in FIG. 12.

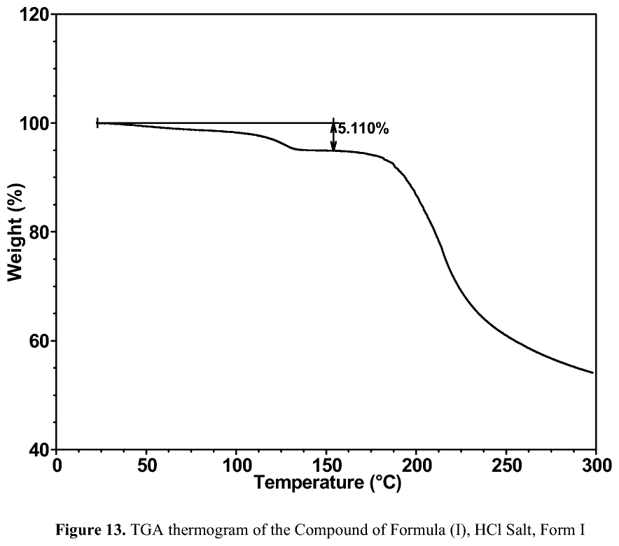

In some embodiments, the compound of Formula (I), HCl salt, Form I is characterized by a TGA pattern substantially as set forth in FIG. 13.

In some embodiments of the compound of Formula (I), HCl salt, Form I, at least one, at least two, at least three, or all of the following (a)-(c) apply: (a) compound of Formula (I), HCl salt, Form I has an XRPD pattern substantially as shown in FIG. 11; (b) compound of Formula (I), HCl salt, Form I has a DSC thermogram substantially as shown in FIG. 12; and (c) compound of Formula (I), HCl salt, Form I has a TGA graph substantially as shown in FIG. 13.

In some embodiments, compound of Formula (I), HCl salt, Form I, has an XRPD pattern displaying at least two, at least three, at least four, at least five, or at least six of the 2θ-reflections as the XRPD pattern substantially as shown in FIG. 11.

In some embodiments, the disclosure provides a crystalline form of an oxalate salt of the compound of Formula (I).

### Compound of Formula (I), Oxalate Salt, Form (I)

In some embodiments, the disclosure provides a crystalline Form I of the oxalate salt of the compound of Formula (I) (“compound of Formula (I), oxalate salt, Form I”). In some embodiments, the compound of Formula (I), oxalate salt, Form I is characterized by an XRPD pattern comprising 2θ-reflections (±0.2 degrees 2θ) at about 6.6°, 17.2°, and 29.5°. In some embodiments, the compound of Formula (I), oxalate salt, Form I is characterized by an XRPD pattern comprising 2θ-reflections (±0.2 degrees 2θ) at about 6.6°, 17.2°, and 29.5°, and one, two, or three of the 2θ-reflections (±0.2 degrees 2θ) at about 6.9°, 13.8°, and 21.8°. In some embodiments, the compound of Formula (I), oxalate salt, Form I is characterized by an XRPD pattern comprising 2θ-reflections (±0.2 degrees 2θ) at about 6.6°, 17.2°, and 29.5°, and one or two of the 2θ-reflections (±0.2 degrees 2θ) at about 6.9°, 13.8°, and 21.8°. In some embodiments, the compound of Formula (I), oxalate salt, Form I is characterized by an XRPD pattern comprising 2θ-reflections (±0.2 degrees 2θ) at about 6.6°, 17.2°, and 29.5°, and one of the 2θ-reflections (±0.2 degrees 2θ) at about 6.9°, 13.8°, and 21.8°. In some embodiments, the compound of Formula (I), oxalate salt, Form I is characterized by an XRPD pattern comprising 2θ-reflections (±0.2 degrees 2θ) at about 6.6°, 17.2°, and 29.5°, and two of the 2θ-reflections (±degrees 2θ) at about 6.9°, 13.8°, and 21.8°. In some embodiments, the compound of Formula (I), oxalate salt, Form I is characterized by an XRPD pattern comprising 2θ-reflections (±0.2 degrees 2θ) at about 6.6°, 6.9°, 13.8°, 17.2°, 21.8°, and 29.5°.

In some embodiments, the compound of Formula (I), oxalate salt, Form I is characterized by an XRPD pattern comprising 2θ-reflections (±0.2 degrees 2θ) at about 6.6°, 6.9°, 13.8°, 17.2°, 21.8°, and 29.5°, and one, two, or three of the 2θ-reflections (±0.2 degrees 2θ) at about 10.2°, 20.7°, and 27.4°. In some embodiments, the compound of Formula (I), oxalate salt, Form I is characterized by an XRPD pattern comprising 2θ-reflections (±0.2 degrees 2θ) at about 6.6°, 6.9°, 13.8°, 17.2°, 21.8°, and 29.5°, and one or two of the 2θ-reflections (±0.2 degrees 2θ) at about 10.2°, 20.7°, and 27.4°. In some embodiments, the compound of Formula (I), oxalate salt, Form I is characterized by an XRPD pattern comprising 2θ-reflections (±0.2 degrees 2θ) at about 6.6°, 6.9°, 13.8°, 17.2°, 21.8°, and 29.5°, and two of the 2θ-reflections (±0.2 degrees 2θ) at about 10.2°, 20.7°, and 27.4°. In some embodiments, the compound of Formula (I), oxalate salt, Form I is characterized by an XRPD pattern comprising 2θ-reflections (±0.2 degrees 2θ) at about 6.6°, 6.9°, 13.8°, 17.2°, 21.8°, and 29.5°, and one of the 2θ-reflections (±0.2 degrees 2θ) at about 10.2°, 20.7°, and 27.4°. In some embodiments, the compound of Formula (I), oxalate salt, Form I is characterized by an XRPD pattern comprising 2θ-reflections (±0.2 degrees 2θ) at about 6.6°, 6.9°, 10.2°, 13.8°, 17.2°, 20.7°, 21.8°, 27.4° and 29.5°.

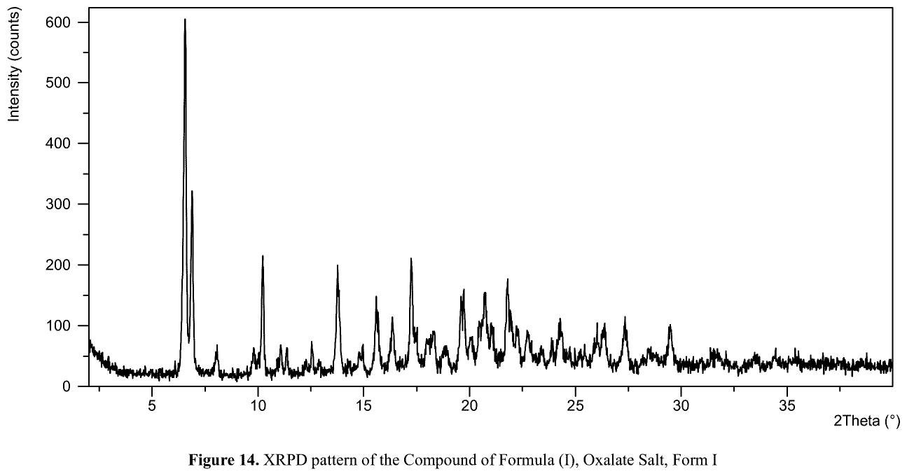

In some embodiments, the compound of Formula (I), oxalate salt, Form I has a XRPD pattern comprising peaks shown in Table 16. In some embodiments, the compound of Formula (I), oxalate salt, Form I has a XRPD pattern substantially as shown in FIG. 14.

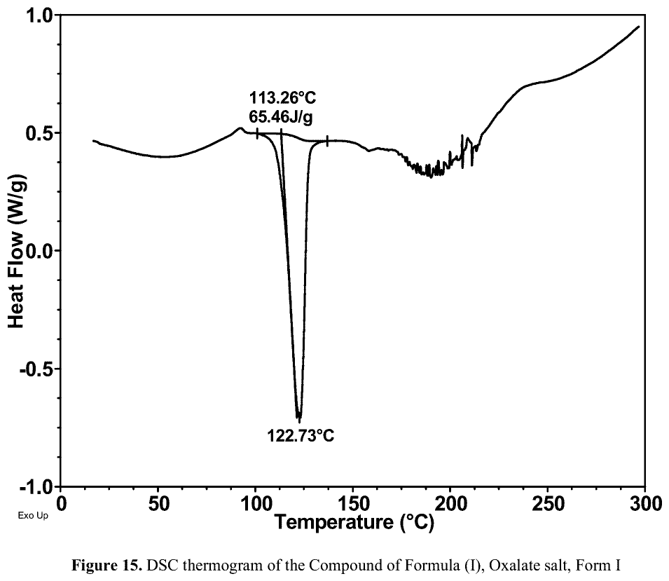

In some embodiments, the compound of Formula (I), oxalate salt, Form I is characterized by a DSC pattern comprising an endothermic transition with onset at about 113° C. In some embodiments, the compound of Formula (I), oxalate salt, Form I is characterized by a DSC pattern substantially as set forth in FIG. 15.

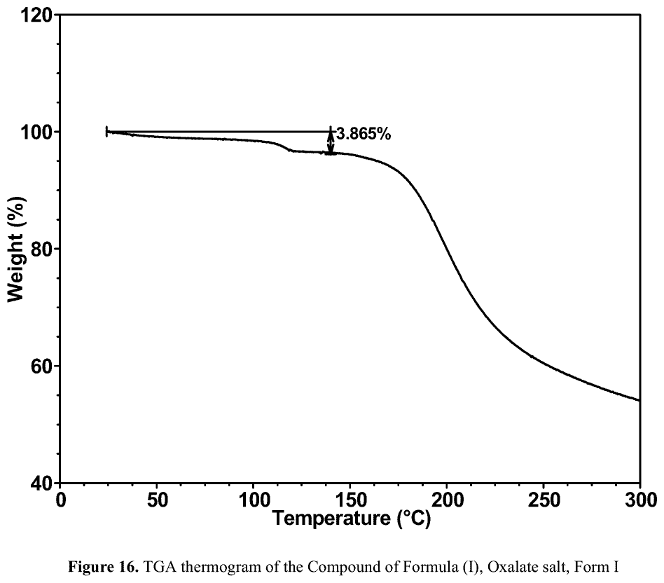

In some embodiments, the compound of Formula (I), oxalate salt, Form I is characterized by a TGA pattern substantially as set forth in FIG. 16.

In some embodiments of the compound of Formula (I), oxalate salt, Form I, at least one, at least two, at least three, or all of the following (a)-(c) apply: (a) compound of Formula (I), oxalate salt, Form I has an XRPD pattern substantially as shown in FIG. 14; (b) compound of Formula (I), oxalate salt, Form I has a DSC thermogram substantially as shown in FIG. 15; and (c) compound of Formula (I), oxalate salt, Form I has a TGA graph substantially as shown in FIG. 16.

In some embodiments, compound of Formula (I), oxalate salt, Form I, has an XRPD pattern displaying at least two, at least three, at least four, at least five, or at least six of the 2θ-reflections as the XRPD pattern substantially as shown in FIG. 14.

### Compound of Formula (I), Oxalate Salt, Form II

In some embodiments, the disclosure provides a crystalline Form II of an oxalate salt of the compound of Formula (I) (“compound of Formula (I), oxalate salt, Form II”). In some embodiments, compound of Formula (I), oxalate salt, Form II is characterized by an XRPD pattern comprising 2θ-reflections (±0.2 degrees 2θ) at about 3.7°, 10.3°, and 26.4°. In some embodiments, compound of Formula (I), oxalate salt, Form II is characterized by an XRPD pattern comprising 2θ-reflections (±0.2 degrees 2θ) at about 3.7°, 10.3°, and 26.4°, and one, two, or three of the 2θ-reflections (±0.2 degrees 2θ) at about 7.3°, 18.2°, and 28.0°. In some embodiments, compound of Formula (I), oxalate salt, Form II is characterized by an XRPD pattern comprising 2θ-reflections (±0.2 degrees 2θ) at about 3.7°, 10.3°, and 26.4°, and one or two of the 2θ-reflections (±0.2 degrees 2θ) at about 7.3°, 18.2°, and 28.0°. In some embodiments, compound of Formula (I), oxalate salt, Form II is characterized by an XRPD pattern comprising 2θ-reflections (±0.2 degrees 2θ) at about 3.7°, 10.3°, and 26.4°, and two of the 2θ-reflections (±0.2 degrees 2θ) at about 7.3°, 18.2°, and 28.0°. In some embodiments, compound of Formula (I), oxalate salt, Form II is characterized by an XRPD pattern comprising 2θ-reflections (±0.2 degrees 2θ) at about 3.7°, 10.3°, and 26.4°, and one of the 2θ-reflections (±0.2 degrees 2θ) at about 7.3°, 18.2°, and 28.0°. In some embodiments, compound of Formula (I), oxalate salt, Form II is characterized by an XRPD pattern comprising 2θ-reflections (±0.2 degrees 2θ) at about 3.7°, 7.3°, 10.3°, 18.2°, 26.4°, and 28.0°.

In some embodiments, compound of Formula (I), oxalate salt, Form II is characterized by an XRPD pattern comprising 2θ-reflections (±0.2 degrees 2θ) at about 3.7°, 7.3°, 10.3°, 18.2°, 26.4°, and 28.0°, and one, two, or three of the 2θ-reflections (±0.2 degrees 2θ) at about 6.4°, 15.0°, 19.6°. In some embodiments, compound of Formula (I), oxalate salt, Form II is characterized by an XRPD pattern comprising 2θ-reflections (±0.2 degrees 2θ) at about 3.7°, 7.3°, 10.3°, 18.2°, 26.4°, and 28.0°, and one or two of the 2θ-reflections (±0.2 degrees 2θ) at about 6.4°, 15.0°, 19.6°. In some embodiments, compound of Formula (I), oxalate salt, Form II is characterized by an XRPD pattern comprising 2θ-reflections (±0.2 degrees 2θ) at about 3.7°, 7.3°, 10.3°, 18.2°, 26.4°, and 28.0°, and two of the 2θ-reflections (±0.2 degrees 2θ) at about 6.4°, 15.0°, 19.6°. In some embodiments, compound of Formula (I), oxalate salt, Form II is characterized by an XRPD pattern comprising 2θ-reflections (±0.2 degrees 2θ) at about 3.7°, 7.3°, 10.3°, 18.2°, 26.4°, and 28.0°, and one of the 2θ-reflections (±0.2 degrees 2θ) at about 6.4°, 15.0°, 19.6°. In some embodiments, compound of Formula (I), oxalate salt, Form II is characterized by an XRPD pattern comprising 2θ-reflections (±0.2 degrees 2θ) at about 3.7°, 6.4°, 7.3°, 10.3°, 15.0°, 18.2°, 19.6°, 26.4°, and 28.0°.

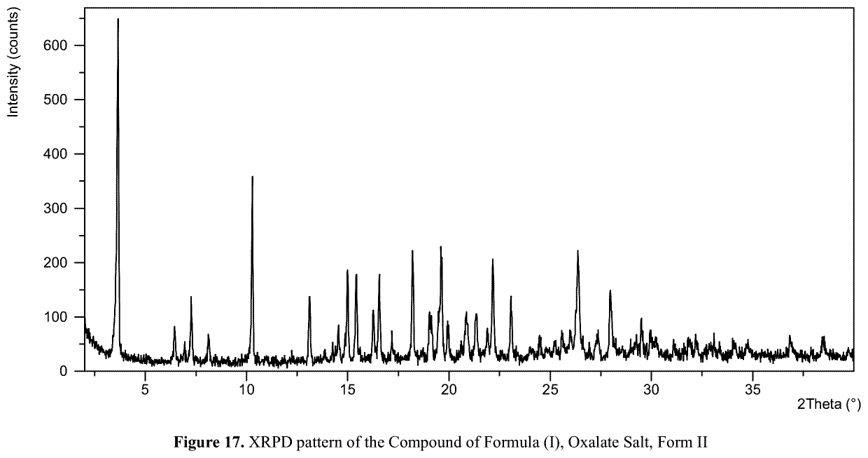

In some embodiments, compound of Formula (I), oxalate salt, Form II is characterized by an XRPD pattern as listed in Table 18. In some embodiments, compound of Formula (I), oxalate salt, Form II is characterized by an XRPD pattern substantially as shown in FIG. 17.

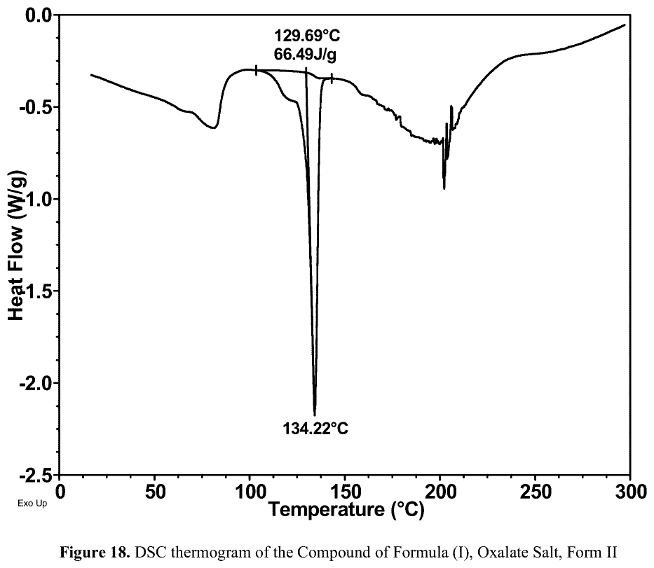

In some embodiments, compound of Formula (I), oxalate salt, Form II is characterized by a DSC pattern comprising an endothermic transition with onset at about 130° C. In some embodiments, compound of Formula (I), oxalate salt, Form II is characterized by a DSC pattern substantially as set forth in FIG. 18.

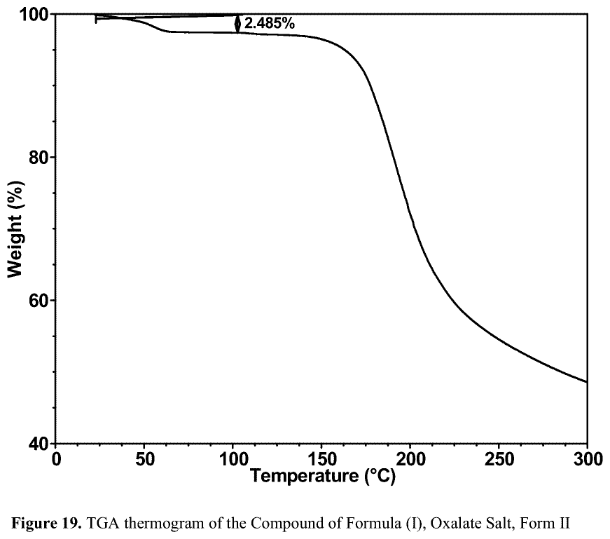

In some embodiments, compound of Formula (I), oxalate salt, Form II is characterized by a TGA pattern substantially as set forth in FIG. 19.

In some embodiments of the compound of Formula (I), oxalate salt, Form II, at least one, at least two, at least three, or all of the following (a)-(c) apply: (a) compound of Formula (I), oxalate salt, Form II has an XRPD pattern substantially as shown in FIG. 17; (b) compound of Formula (I), oxalate salt, Form II has a DSC thermogram substantially as shown in FIG. 18; and (c) compound of Formula (I), oxalate salt, Form II has a TGA graph substantially as shown in FIG. 19.

In some embodiments, compound of Formula (I), oxalate salt, Form I, has an XRPD pattern displaying at least two, at least three, at least four, at least five, or at least six of the 2θ-reflections as the XRPD pattern substantially as shown in FIG. 17.

In some embodiments, the present disclosure provides a crystalline form of a maleate salt of the compound of Formula I.

### Compound of Formula (I), Maleate Salt, Form I

In some embodiments, the present disclosure provides a crystalline Form I of a maleate salt of the compound of Formula I (“compound of Formula (I), maleate salt, Form I”). In some embodiments, the compound of Formula (I), maleate salt, Form I is characterized by an XRPD pattern comprising 2θ-reflections (±0.2 degrees 2θ) at about 3.9°, 7.8°, and 19.7°. In some embodiments, the compound of Formula (I), maleate salt, Form I is characterized by an XRPD pattern comprising 2θ-reflections (±0.2 degrees 2θ) at about 3.9°, 7.8°, and 19.7°, and one or two of the 2θ-reflections (±0.2 degrees 2θ) at about 11.8° and 23.7°. In some embodiments, the compound of Formula (I), maleate salt, Form I is characterized by an XRPD pattern comprising 2θ-reflections (±0.2 degrees 2θ) at about 3.9°, 7.8°, and 19.7°, and one of the 2θ-reflections (±0.2 degrees 2θ) at about 11.8° and 23.7°. In some embodiments, the compound of Formula (I), maleate salt, Form I is characterized by an XRPD pattern comprising 2θ-reflections (±0.2 degrees 2θ) at about 3.9°, 7.8°, 11.8°, 19.7°, and 23.7°.

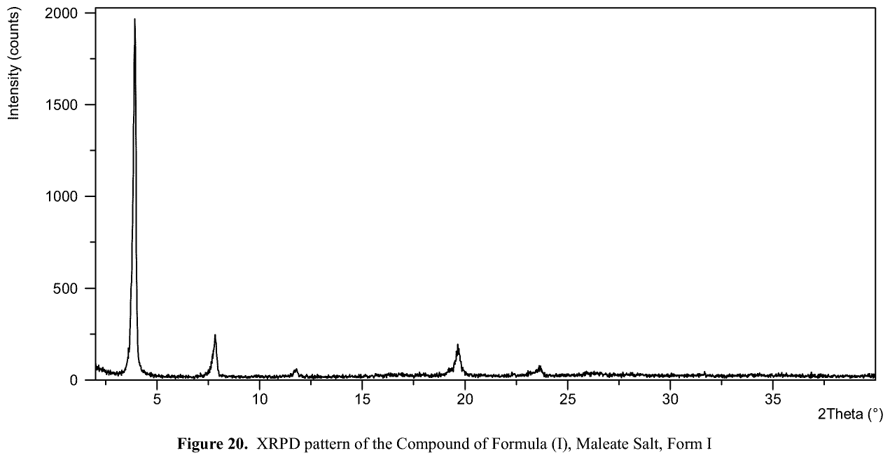

In some embodiments, the compound of Formula (I), maleate salt, Form I is characterized by an XRPD pattern substantially as shown in FIG. 20. In some embodiments, the compound of Formula (I), maleate salt, Form I is characterized by an XRPD pattern comprising peaks as listed in Table 20.

### Compound of Formula (I), Maleate Salt, Form II

In some embodiments, the present disclosure provides a crystalline Form II of a maleate salt of the compound of Formula I (“compound of Formula (I), maleate salt, Form II”). In some embodiments, the compound of Formula (I), maleate salt, Form II is characterized by an XRPD pattern comprising 2θ-reflections (±0.2 degrees 2θ) at about 3.9°, 7.7°, and 16.9°. In some embodiments, the compound of Formula (I), maleate salt, Form II is characterized by an XRPD pattern comprising 2θ-reflections (±0.2 degrees 2θ) at about 3.9°, 7.7°, and 16.9°, and one, two, or three of the 2θ-reflections (±0.2 degrees 2θ) at about 10.4°, 18.0°, and 25.9°. In some embodiments, the compound of Formula (I), maleate salt, Form II is characterized by an XRPD pattern comprising 2θ-reflections (±0.2 degrees 2θ) at about 3.9°, 7.7°, and 16.9°, and one or two of the 2θ-reflections (±0.2 degrees 2θ) at about 10.4°, 18.0°, and 25.9°. In some embodiments, the compound of Formula (I), maleate salt, Form II is characterized by an XRPD pattern comprising 2θ-reflections (±0.2 degrees 2θ) at about 3.9°, 7.7°, and 16.9°, and one of the 2θ-reflections (±0.2 degrees 2θ) at about 10.4°, 18.0°, and 25.9°. In some embodiments, the compound of Formula (I), maleate salt, Form II is characterized by an XRPD pattern comprising (±0.2 degrees 2θ) at about 3.9°, 7.7°, and 16.9°, and two of the 2θ-reflections (±0.2 degrees 2θ) at about 10.4°, 18.0°, and 25.9°. In some embodiments, the compound of Formula (I), maleate salt, Form II is characterized by an XRPD pattern comprising 2θ-reflections (±0.2 degrees 2θ) at about 3.9°, 7.7°, 10.4°, 16.9°, 18.0°, and 25.9°.

In some embodiments, the compound of Formula (I), maleate salt, Form II is characterized by an XRPD pattern comprising 2θ-reflections (±0.2 degrees 2θ) at about 3.9°, 7.7°, 10.4°, 16.9°, 18.0°, and 25.9°, and one, two, or three of the 2θ-reflections (±0.2 degrees 2θ) at about 15.8°, 19.2°, and 26.8°. In some embodiments, the compound of Formula (I), maleate salt, Form II is characterized by an XRPD pattern comprising 2θ-reflections (±0.2 degrees 2θ) at about 3.9°, 7.7°, 10.4°, 16.9°, 18.0°, and 25.9°, and one or two of the 2θ-reflections (±0.2 degrees 2θ) at about 15.8°, 19.2°, and 26.8°. In some embodiments, the compound of Formula (I), maleate salt, Form II is characterized by an XRPD pattern comprising 2θ-reflections (±0.2 degrees 2θ) at about 3.9°, 7.7°, 10.4°, 16.9°, 18.0°, and 25.9°, and one of the 2θ-reflections (±0.2 degrees 2θ) at about 15.8°, 19.2°, and 26.8°. In some embodiments, the compound of Formula (I), maleate salt, Form II is characterized by an XRPD pattern comprising 2θ-reflections (±0.2 degrees 2θ) at about 3.9°, 7.7°, 10.4°, 16.9°, 18.0°, and 25.9°, and two of the 2θ-reflections (±0.2 degrees 2θ) at about 15.8°, 19.2°, and 26.8°. In some embodiments, the compound of Formula (I), maleate salt, Form II is characterized by an XRPD pattern comprising 2θ-reflections (±0.2 degrees 2θ) at about 3.9°, 7.7°, 10.4°, 15.8°, 16.9°, 18.0°, 19.2°, 25.9° and 26.8°.

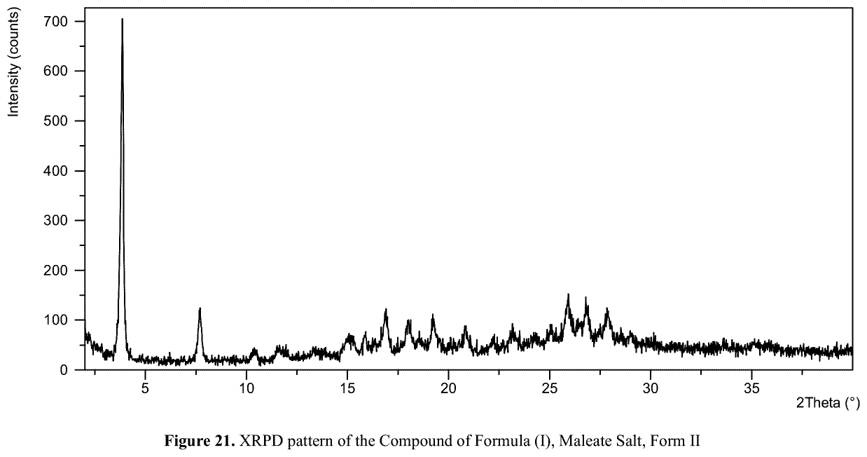

In some embodiments, the compound of Formula (I), maleate salt, Form II is characterized by an XRPD pattern substantially as shown in FIG. 21. In some embodiments, the compound of Formula (I), maleate salt, Form II is characterized by an XRPD pattern substantially as listed in Table 24.

### Compound of Formula (I), Maleate Salt, Form III

In some embodiments, the current disclosure provides a crystalline Form III of the maleate salt of the compound of Formula (I) (“compound (I), maleate salt, Form III”). In some embodiments, compound (I), maleate salt, Form III is characterized by an XRPD pattern comprising 2θ-reflections (±0.2 degrees 2θ) at about 3.8°, 7.6°, and 19.2°. In some embodiments, compound (I), maleate salt, Form III is characterized by an XRPD pattern comprising 2θ-reflections (±0.2 degrees 2θ) at about 3.8°, 7.6°, and 19.2°, and one, two, or three of the 2θ-reflections (±0.2 degrees 2θ) at about 11.5°, 16.8°, and 23.1°. In some embodiments, compound (I), maleate salt, Form III is characterized by an XRPD pattern comprising 2θ-reflections (±0.2 degrees 2θ) at about 3.8°, 7.6°, and 19.2°, and one or two of the (±0.2 degrees 2θ) at about 11.5°, 16.8°, and 23.1°. In some embodiments, compound (I), maleate salt, Form III is characterized by an XRPD pattern comprising 2θ-reflections (±0.2 degrees 2θ) at about 3.8°, 7.6°, and 19.2°, and two of the 2θ-reflections (±0.2 degrees 2θ) at about 11.5°, 16.8°, and 23.1°. In some embodiments, compound (I), maleate salt, Form III is characterized by an XRPD pattern comprising 2θ-reflections (±0.2 degrees 2θ) at about 3.8°, 7.6°, and 19.2°, and one of the 2θ-reflections (±0.2 degrees 2θ) at about 11.5°, 16.8°, and 23.1°. In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°.

In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°, and one, two, or three of the 2θ-reflections (±0.2 degrees 2θ) at about 10.4°, and 27.5°. In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°, and one or two of the 2θ-reflections (±0.2 degrees 2θ) at about 10.4°, 15.1°, and 27.5°. In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°, and two of the 2θ-reflections (±0.2 degrees 2θ) at about 10.4°, 15.1°, and 27.5°. In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°, and one of the 2θ-reflections (±0.2 degrees 2θ) at about 10.4°, 15.1°, and 27.5°. In some embodiments, compound (I), maleate salt, Form III is characterized by an XRPD pattern comprising 2θ-reflections (±0.2 degrees 2θ) at about 3.8°, 7.6°, 11.5°, 16.8°, 19.2°, and 23.1°, and one, two, or three of the 2θ-reflections (±0.2 degrees 2θ) at about 3.8°, 7.6°, 10.4°, 11.5°, 16.8°, 19.2°, 23.1°, and 27.5°.

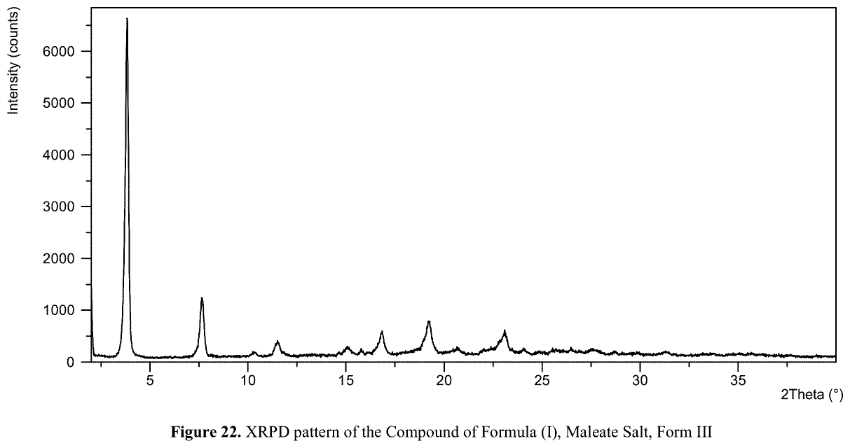

In some embodiments, compound (I), maleate salt, Form III is characterized by an XRPD pattern substantially as shown in FIG. 22. In some embodiments, compound (I), maleate salt, Form III is characterized by an XRPD pattern as listed in Table 26.

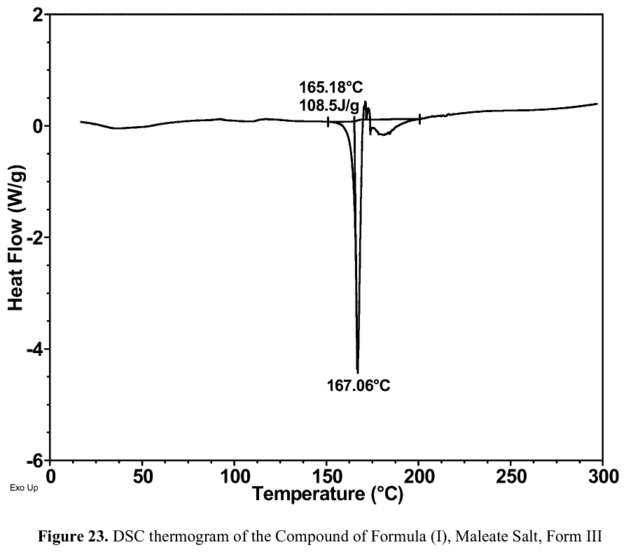

In some embodiments, compound (I), maleate salt, Form III is characterized by an XRPD pattern is characterized by a DSC pattern comprising an endothermic transition with onset at about 165° C. In some embodiments, compound (I), maleate salt, Form III is characterized by an XRPD pattern is characterized by a DSC pattern substantially as set forth in FIG. 23.

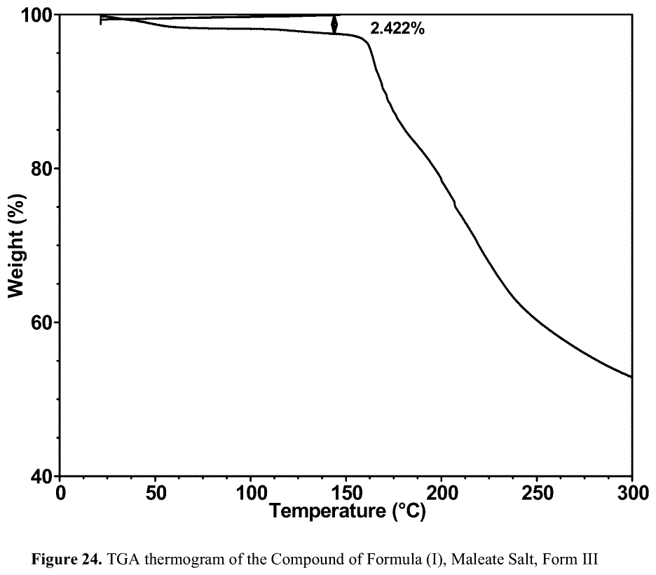

In some embodiments, compound (I), maleate salt, Form III is characterized by an XRPD pattern is characterized by a TGA pattern substantially as set forth in FIG. 24.

### Compound of Formula (I), Maleate Salt, Form IV

In some embodiments, the current disclosure provides a crystalline Form IV of the maleate salt of the compound of Formula (I) (“compound of Formula (I), maleate salt, Form IV”). In some embodiments, compound of Formula (I), maleate salt, Form IV is characterized by an XRPD pattern comprising 2θ-reflections (±0.2 degrees 2θ) at about 8.8°, 15.0°, and 25.5°.

In some embodiments, compound of Formula (I), maleate salt, Form IV is characterized by an XRPD pattern comprising 2θ-reflections (±0.2 degrees 2θ) at about 8.8°, and 25.5°, and one, two, or three of the 2θ-reflections (±0.2 degrees 2θ) at about 15.0°, 21.4°, and 30.3°. In some embodiments, compound of Formula (I), maleate salt, Form IV is characterized by an XRPD pattern comprising 2θ-reflections (±0.2 degrees 2θ) at about 8.8°, and 25.5°, and one or two of the 2θ-reflections (±0.2 degrees 2θ) at about 15.0°, 21.4°, and 30.3°. In some embodiments, compound of Formula (I), maleate salt, Form IV is characterized by an XRPD pattern comprising 2θ-reflections (±0.2 degrees 2θ) at about 8.8°, and 25.5°, and one of the 2θ-reflections (±0.2 degrees 2θ) at about 15.0°, 21.4°, and 30.3°. In some embodiments, compound of Formula (I), maleate salt, Form IV is characterized by an XRPD pattern comprising 2θ-reflections (±0.2 degrees 2θ) at about 8.8°, 15.0°, and 25.5°, and two of the 2θ-reflections (±0.2 degrees 2θ) at about 15.0°, 21.4°, and 30.3°. In some embodiments, compound of Formula (I), maleate salt, Form IV is characterized by an XRPD pattern comprising 2θ-reflections (±0.2 degrees 2θ) at about 5.4°, 8.8°, 15.0°, 21.4°, 25.5°, and

In some embodiments, compound of Formula (I), maleate salt, Form IV is characterized by an XRPD pattern comprising 2θ-reflections (±0.2 degrees 2θ) at about 5.4°, 8.8°, 15.0°, 21.4°, 25.5°, and 30.3°, and one, two, or three of the 2θ-reflections (±0.2 degrees 2θ) at about 14.0°, 17.6°, and 35.7°. In some embodiments, compound of Formula (I), maleate salt, Form IV is characterized by an XRPD pattern comprising 2θ-reflections (±0.2 degrees 2θ) at about 5.4°, 8.8°, 15.0°, 21.4°, 25.5°, and 30.3°, and one or two of the 2θ-reflections (±0.2 degrees 2θ) at about 14.0°, 17.6°, and 35.7°. In some embodiments, compound of Formula (I), maleate salt, Form IV is characterized by an XRPD pattern comprising 2θ-reflections (±0.2 degrees 2θ) at about 5.4°, 8.8°, 15.0°, 21.4°, 25.5°, and 30.3°, and two of the 2θ-reflections (±0.2 degrees 2θ) at about 14.0°, 17.6°, and 35.7°. In some embodiments, compound of Formula (I), maleate salt, Form IV is characterized by an XRPD pattern comprising 2θ-reflections (±0.2 degrees 2θ) at about 5.4°, 8.8°, 15.0°, 21.4°, 25.5°, and 30.3°, and one of the 2θ-reflections (±0.2 degrees 2θ) at about 14.0°, 17.6°, and 35.7°. In some embodiments, compound of Formula (I), maleate salt, Form IV is characterized by an XRPD pattern comprising 2θ-reflections (±0.2 degrees 2θ) at about 5.4°, 8.8°, 14.0°, 15.0°, 17.6°, 21.4°, 25.5° and 30.3°, and 35.7°.

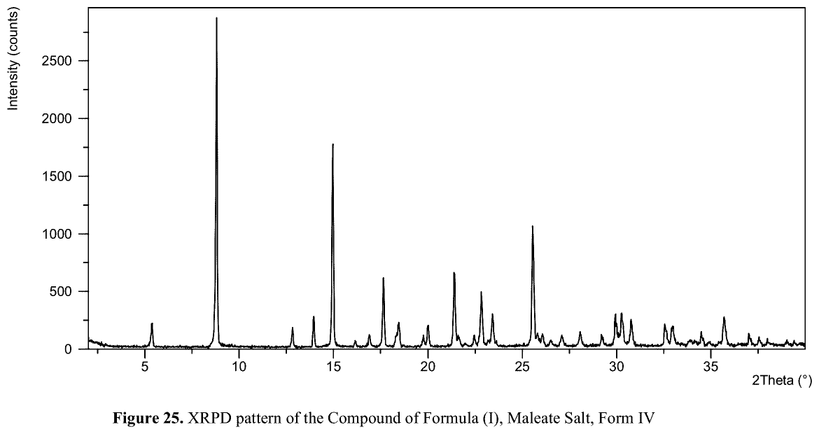

In some embodiments, compound of Formula (I), maleate salt, Form IV is characterized by an XRPD pattern substantially as shown in FIG. 25. In some embodiments, compound of Formula (I), maleate salt, Form IV is characterized by an XRPD pattern as listed in Table 8.

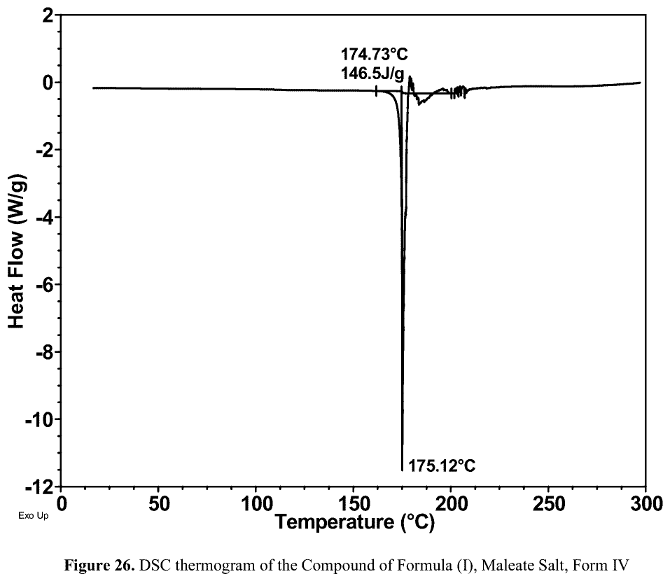

In some embodiments, compound of Formula (I), maleate salt, Form IV is characterized by a DSC pattern comprising an endothermic transition with onset at about 175° C. In some embodiments, compound of Formula (I), maleate salt, Form IV is characterized by a DSC pattern substantially as set forth in FIG. 26.

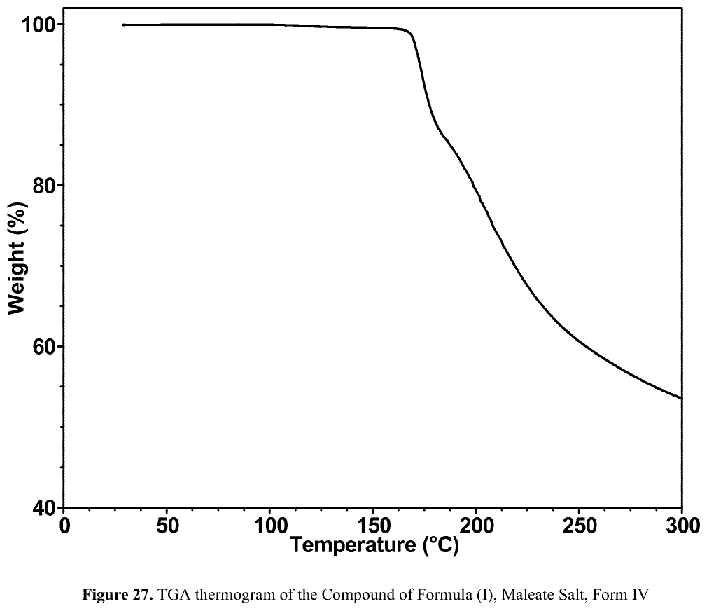

In some embodiments, the compound of Formula (I), maleate salt, Form IV is characterized by a TGA pattern substantially as set forth in FIG. 27.

In some embodiments, the current disclosure provides a solvate of a salt of the compound of Formula (I). In some embodiments, the current disclosure provides a solvate of the maleate salt of the compound of Formula (I). In some embodiments, the current disclosure provides an acetonitrile solvate (MeCN) of the maleate salt of the compound of Formula (I).

### Compound of Formula (I), Maleate Salt, MeCN Solvate I

In some embodiments, the disclosure provides a crystalline form of the acetonitrile solvate of the maleate salt of the compound of Formula (I). In some embodiments, the disclosure provides a crystalline form I of the acetonitrile solvate of the maleate salt of the compound of Formula (I) (“compound of Formula (I), maleate salt, MeCN solvate I”).

In some embodiments, the compound of Formula (I), maleate salt, MeCN solvate I is characterized by an XRPD pattern comprising 2θ-reflections (±0.2 degrees 2θ) at about 3.5°, 7.0°, and 20.9°. In some embodiments, the compound of Formula (I), maleate salt, MeCN solvate I is characterized by an XRPD pattern comprising 2θ-reflections (±0.2 degrees 2θ) at about 3.5°, 7.0°, and 20.9°, and one, two, or three of the 2θ-reflections (±0.2 degrees 2θ) at about 3.7°, 7.3°, and 22.0°. In some embodiments, the compound of Formula (I), maleate salt, MeCN solvate I is characterized by an XRPD pattern comprising 2θ-reflections (±0.2 degrees 2θ) at about 3.5°, 7.0°, and 20.9°, and one or two of the 2θ-reflections (±0.2 degrees 2θ) at about 3.7°, 7.3°, and 22.0°. In some embodiments, the compound of Formula (I), maleate salt, MeCN solvate I is characterized by an XRPD pattern comprising 2θ-reflections (±0.2 degrees 2θ) at about 3.5°, 7.0°, and 20.9°, and one of the 2θ-reflections (±0.2 degrees 2θ) at about 3.7°, 7.3°, and 22.0°. In some embodiments, the compound of Formula (I), maleate salt, MeCN solvate I is characterized by an XRPD pattern comprising 2θ-reflections (±0.2 degrees 2θ) at about 3.5°, 7.0°, and 20.9°, and two of the 2θ-reflections (±0.2 degrees 2θ) at about 3.7°, 7.3°, and 22.0°. In some embodiments, the compound of Formula (I), maleate salt, MeCN solvate I is characterized by an XRPD pattern comprising 2θ-reflections (±0.2 degrees 2θ) at about 3.5°, 3.7°, 7.0°, 7.3°, 20.9°, and 22.0°.

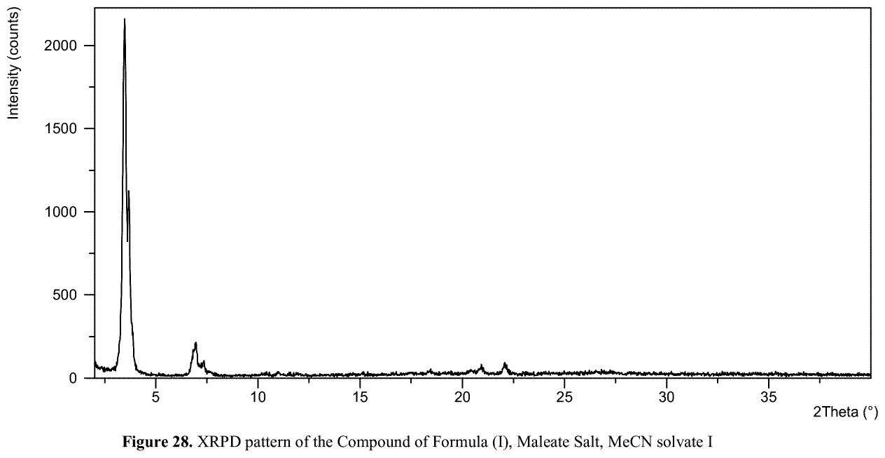

In some embodiments, the compound of Formula (I), maleate salt, MeCN solvate I is characterized by an XRPD pattern substantially as shown in FIG. 28. In some embodiments, the compound of Formula (I), maleate salt, MeCN solvate I is characterized by an XRPD pattern listed in Table 18.

### Compound of Formula (I), Maleate Salt, MeCN Solvate II

In some embodiments, the disclosure provides a crystalline form II of the acetonitrile solvate of the maleate salt of the compound of Formula (I) (“compound of Formula (I), maleate salt, MeCN solvate II”).

In some embodiments, the compound of Formula (I), maleate salt, MeCN solvate II is characterized by an XRPD pattern comprising 2θ-reflections (±0.2 degrees 2θ) at about 3.4°, 6.8°, and 25.2°. In some embodiments, the compound of Formula (I), maleate salt, MeCN solvate II is characterized by an XRPD pattern comprising 2θ-reflections (±0.2 degrees 2θ) at about 3.4°, 6.8°, and 25.2°, and one, two, or three of the 2θ-reflections (±0.2 degrees 2θ) at about 3.7°, 14.8°, and 27.6°. In some embodiments, the compound of Formula (I), maleate salt, MeCN solvate II is characterized by an XRPD pattern comprising 2θ-reflections (±0.2 degrees 2θ) at about 3.4°, 6.8°, and 25.2°, and one or two of the 2θ-reflections (±0.2 degrees 2θ) at about 3.7°, 14.8°, and 27.6°. In some embodiments, the compound of Formula (I), maleate salt, MeCN solvate II is characterized by an XRPD pattern comprising 2θ-reflections (±0.2 degrees 2θ) at about 3.4°, 6.8°, and 25.2°, and one of the 2θ-reflections (±0.2 degrees 2θ) at about 3.7°, 14.8°, and 27.6°. In some embodiments, the compound of Formula (I), maleate salt, MeCN solvate II is characterized by an XRPD pattern comprising 2θ-reflections (±0.2 degrees 2θ) at about 3.4°, 6.8°, and 25.2°, and two of the 2θ-reflections (±0.2 degrees 2θ) at about 3.7°, 14.8°, and 27.6°. In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 25.2° and 27.6°.

In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 25.2° and 27.6°, and one, two, or three of the 2θ-reflections (±0.2 degrees 2θ) at about 16.8°, 20.5°, and 26.7°. In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 25.2° and 27.6°, and one or two of the 2θ-reflections (±0.2 degrees 2θ) at about 16.8°, 20.5°, and 26.7°. In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 25.2° and 27.6°, and one of the 2θ-reflections (±0.2 degrees 2θ) at about 16.8°, 20.5°, and 26.7°. In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 25.2° and 27.6°, and two of the 2θ-reflections (±0.2 degrees 2θ) at about 16.8°, 20.5°, and 26.7°. In some embodiments, the compound of Formula (I), maleate salt, MeCN solvate II is characterized by an XRPD pattern comprising 2θ-reflections (±0.2 degrees 2θ) at about 3.4°, 3.7°, 6.8°, 14.8°, 16.8°, 20.5°, 25.2°, 26.7°, and 27.6°.

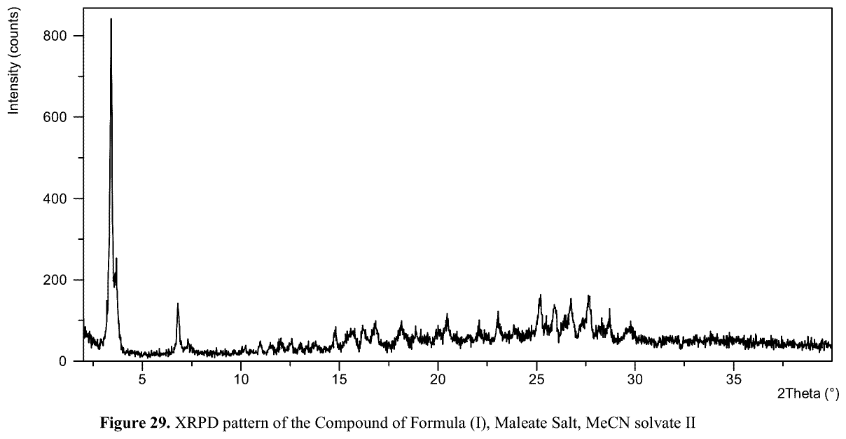

In some embodiments, the compound of Formula (I), maleate salt, MeCN solvate II is characterized by an XRPD pattern substantially as shown in FIG. 29. In some embodiments, the compound of Formula (I), maleate salt, MeCN solvate II is characterized by an XRPD pattern as listed in Table 22.

In some embodiments, the disclosure provides a mesylate salt of the compound of Formula (I). In some embodiments, the disclosure provides a crystalline form of the mesylate salt of the compound of Formula (I).

### Compound of Formula (I), Mesylate Salt, Form I

In some embodiments, the present disclosure provides a crystalline form of the mesylate salt of the compound of Formula I. In some embodiments, the disclosure provides a solvate of the mesylate salt of the compound of Formula (I). In some embodiments, the disclosure provides a crystalline form of the solvate of the mesylate salt of the compound of Formula (I).

### Compound of Formula (I), Mesylate Salt, IPA Solvate

In some embodiments, the disclosure provides an IPA (isopropyl alcohol) solvate of the mesylate salt of the compound of Formula (I) (“compound of Formula (I), mesylate salt, IPA solvate. In some embodiments, compound of Formula (I), mesylate salt, IPA solvate is crystalline.

In some embodiments, the present disclosure provides crystalline form of compound of Formula (I), mesylate salt, IPA solvate characterized by an XRPD pattern comprising 2θ-reflections (±0.2 degrees 2θ) at about 7.5°, 18.2°, and 25.0°. In some embodiments, the crystalline form of compound of Formula (I), mesylate salt, IPA solvate characterized by an XRPD pattern comprising 2θ-reflections (±0.2 degrees 2θ) at about 7.5°, 18.2°, and 25.0°, and one, two, or three of the 2θ-reflections (±0.2 degrees 2θ) at about 8.7°, 19.7°, and 29.7°. In some embodiments, the crystalline form of compound of Formula (I), mesylate salt, IPA solvate characterized by an XRPD pattern comprising 2θ-reflections (±0.2 degrees 2θ) at about 7.5°, 18.2°, and 25.0°, and one or two of the 2θ-reflections (±0.2 degrees 2θ) at about 8.7°, 19.7°, and 29.7°. In some embodiments, the crystalline form of compound of Formula (I), mesylate salt, IPA solvate characterized by an XRPD pattern comprising 2θ-reflections (±0.2 degrees 2θ) at about 7.5°, 18.2°, and 25.0°, and one of the 2θ-reflections (±0.2 degrees 2θ) at about 8.7°, 19.7°, and 29.7°. In some embodiments, the crystalline form of compound of Formula (I), mesylate salt, IPA solvate characterized by an XRPD pattern comprising 2θ-reflections (±0.2 degrees 2θ) at about 7.5°, 18.2°, and 25.0°, and two of the 2θ-reflections (±0.2 degrees 2θ) at about 8.7°, 19.7°, and 29.7°. In some embodiments, the crystalline form of compound of Formula (I), mesylate salt, IPA solvate characterized by an XRPD pattern comprising 2θ-reflections (±0.2 degrees 2θ) at about 7.5°, 8.7°, 18.2°, 19.7°, 25.0°, and 29.7°.

In some embodiments, the crystalline form of compound of Formula (I), mesylate salt, IPA solvate characterized by an XRPD pattern comprising 2θ-reflections (±0.2 degrees 2θ) at about 7.5°, 8.7°, 18.2°, 19.7°, 25.0°, and 29.7°, and one, two, or three of the 2θ-reflections (±0.2 degrees 2θ) at about 9.8°, 16.4°, and 27.2°. In some embodiments, the crystalline form of compound of Formula (I), mesylate salt, IPA solvate characterized by an XRPD pattern comprising 2θ-reflections (±0.2 degrees 2θ) at about 7.5°, 8.7°, 18.2°, 19.7°, 25.0°, and 29.7°, and one or two of the 2θ-reflections (±0.2 degrees 2θ) at about 9.8°, 16.4°, and 27.2°. In some embodiments, the crystalline form of compound of Formula (I), mesylate salt, IPA solvate characterized by an XRPD pattern comprising 2θ-reflections (±0.2 degrees 2θ) at about 7.5°, 8.7°, 18.2°, 19.7°, 25.0°, and 29.7°, and one of the 2θ-reflections (±0.2 degrees 2θ) at about 9.8°, 16.4°, and 27.2°. In some embodiments, the crystalline form of compound of Formula (I), mesylate salt, IPA solvate characterized by an XRPD pattern comprising 2θ-reflections (±0.2 degrees 2θ) at about 7.5°, 8.7°, 18.2°, 19.7°, 25.0°, and 29.7°, and two of the 2θ-reflections (±0.2 degrees 2θ) at about 9.8°, 16.4°, and 27.2°. In some embodiments, the crystalline form of compound of Formula (I), mesylate salt, IPA solvate characterized by an XRPD pattern comprising 2θ-reflections (±0.2 degrees 2θ) at about 7.5°, 8.7°, 9.8°, 16.4°, 18.2°, 19.7°, 25.0°, 27.2°, and 29.7°.

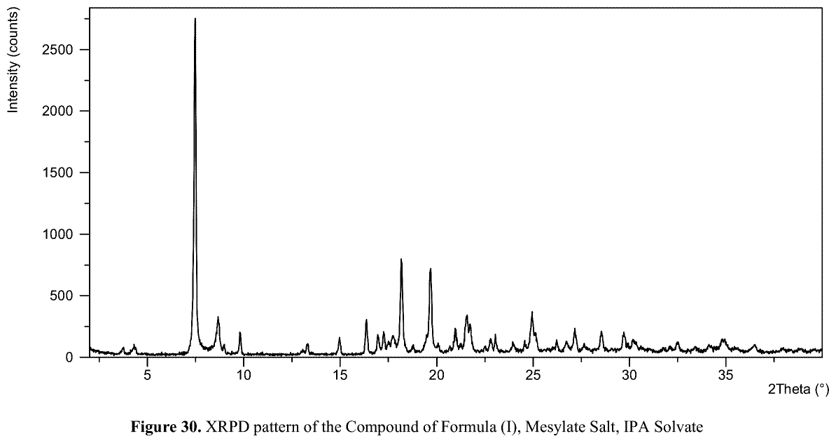

In some embodiments, the crystalline form of compound of Formula (I), mesylate salt, IPA solvate characterized by an XRPD pattern is substantially as shown in FIG. 30. In some embodiments, the crystalline form of compound of Formula (I), mesylate salt, IPA solvate characterized by an XRPD pattern as listed in Table 32.

### Compound of Formula (I), Mesylate Salt, Hydrate

In some embodiments, the present disclosure provides a hydrate of the mesylate salt of the compound of Formula (I) (“compound of Formula I, mesylate salt, hydrate”). In some embodiments, compound of Formula I, mesylate salt, hydrate is crystalline.

In some embodiments, the crystalline form of compound of Formula I, mesylate salt, hydrate is characterized by an XRPD pattern comprising 2θ-reflections (±0.2 degrees 2θ) at about 8.7°, 17.7°, and 24.8°. In some embodiments, the crystalline form of compound of Formula I, mesylate salt, hydrate is characterized by an XRPD pattern comprising 2θ-reflections (±0.2 degrees 2θ) at about 8.7°, 17.7°, and 24.8°, and one, two, or three of the 2θ-reflections (±0.2 degrees 2θ) at about 4.4°, 19.4°, and 28.2°. In some embodiments, the crystalline form of compound of Formula I, mesylate salt, hydrate is characterized by an XRPD pattern comprising 2θ-reflections (±0.2 degrees 2θ) at about 8.7°, 17.7°, and 24.8°, and one or two of the 2θ-reflections (±0.2 degrees 2θ) at about 4.4°, 19.4°, and 28.2°. In some embodiments, the crystalline form of compound of Formula I, mesylate salt, hydrate is characterized by an XRPD pattern comprising 2θ-reflections (±0.2 degrees 2θ) at about 8.7°, 17.7°, and 24.8°, and one of the 2θ-reflections (±0.2 degrees 2θ) at about 4.4°, 19.4°, and 28.2°. In some embodiments, the crystalline form of compound of Formula I, mesylate salt, hydrate is characterized by an XRPD pattern comprising 2θ-reflections (±0.2 degrees 2θ) at about 8.7°, 17.7°, and 24.8°, and two of the 2θ-reflections (±0.2 degrees 2θ) at about 4.4°, 19.4°, and 28.2°. In some embodiments, the crystalline form of compound of Formula I, mesylate salt, hydrate is characterized by an XRPD pattern comprising 2θ-reflections (±0.2 degrees 2θ) at about 4.4°, 8.7°, 17.7°, 19.4°, 24.8°, and 28.2°.

In some embodiments, the crystalline form of compound of Formula I, mesylate salt, hydrate is characterized by an XRPD pattern comprising 2θ-reflections (±0.2 degrees 2θ) at about 4.4°, 8.7°, 17.7°, 19.4°, 24.8°, and 28.2°, and one, two, or three of the 2θ-reflections (±0.2 degrees 2θ) at about 13.1°, 21.1°, and 30.2°. In some embodiments, the crystalline form of compound of Formula I, mesylate salt, hydrate is characterized by an XRPD pattern comprising 2θ-reflections (±0.2 degrees 2θ) at about 4.4°, 8.7°, 17.7°, 19.4°, 24.8°, and 28.2°, and one or two of the 2θ-reflections (±0.2 degrees 2θ) at about 13.1°, 21.1°, and 30.2°. In some embodiments, the crystalline form of compound of Formula I, mesylate salt, hydrate is characterized by an XRPD pattern comprising 2θ-reflections (±0.2 degrees 2θ) at about 4.4°, 8.7°, 17.7°, 19.4°, 24.8°, and 28.2°, and one of the 2θ-reflections (±0.2 degrees 2θ) at about 13.1°, 21.1°, and 30.2°. In some embodiments, the crystalline form of compound of Formula I, mesylate salt, hydrate is characterized by an XRPD pattern comprising 2θ-reflections (±0.2 degrees 2θ) at about 4.4°, 8.7°, 17.7°, 19.4°, 24.8°, and 28.2°, and two of the 2θ-reflections (±0.2 degrees 2θ) at about 13.1°, 21.1°, and 30.2°. In some embodiments, the crystalline form of compound of Formula I, mesylate salt, hydrate is characterized by an XRPD pattern comprising 2θ-reflections (±0.2 degrees 2θ) at about 4.4°, 8.7°, 13.1°, 17.7°, 19.4°, 21.1°, 24.8°, 28.2° and 30.2°.

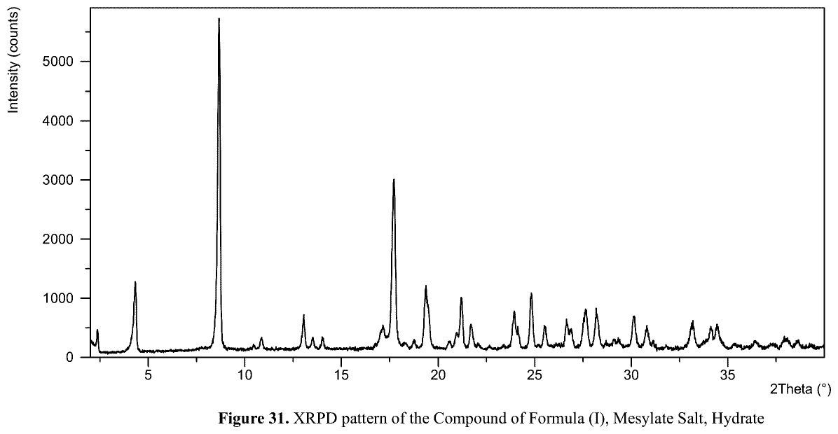

In some embodiments, the crystalline form of compound of Formula I, mesylate salt, hydrate is characterized by an XRPD pattern substantially as shown in FIG. 31. In some embodiments, the crystalline form of compound of Formula I, mesylate salt, hydrate is characterized by an XRPD pattern as listed in Table 30.

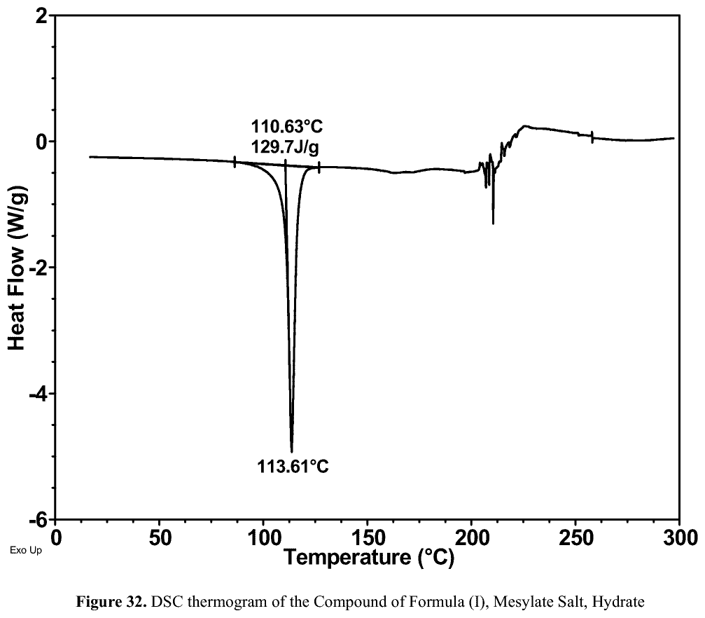

In some embodiments, the crystalline form of compound of Formula I, mesylate salt, hydrate is characterized by a DSC pattern comprising an endothermic transition with onset at about 111° C. In some embodiments, the crystalline form of compound of Formula I, mesylate salt, hydrate is characterized by DSC pattern substantially as set forth in FIG. 32.

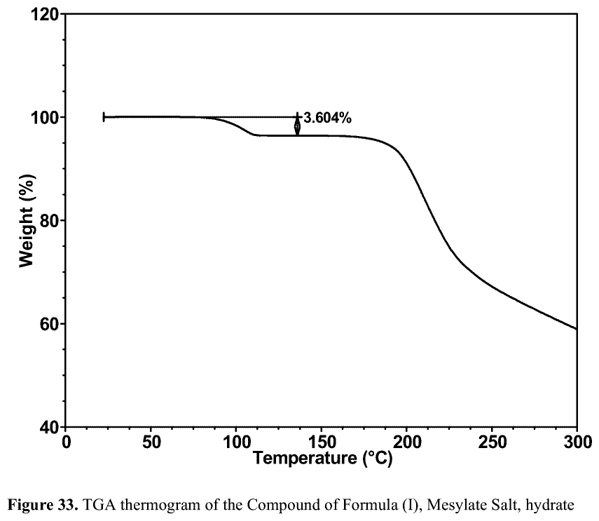

In some embodiments, the crystalline form of compound of Formula I, mesylate salt, hydrate is characterized by a TGA pattern substantially as set forth in FIG. 33.

### Compound of Formula (I), Mesylate Salt, Form I

In some embodiments, the current disclosure provides a crystalline Form I of the mesylate salt of the compound of Formula (I) (“compound of Formula (I), mesylate salt, Form I″). In some embodiments, the compound of Formula (I), mesylate salt, Form I is characterized by an XRPD pattern comprising 2θ-reflections (±0.2 degrees 2θ) at about at 6.3°, 13.8°, and 21.6°. In some embodiments, the compound of Formula (I), mesylate salt, Form I is characterized by an XRPD pattern comprising 2θ-reflections (±0.2 degrees 2θ) at about at 6.3°, 13.8°, and 21.6°, and one, two, or three of the 2θ-reflections (±0.2 degrees 2θ) at about 12.7°, 19.1°, and 24.5°. In some embodiments, the compound of Formula (I), mesylate salt, Form I is characterized by an XRPD pattern comprising 2θ-reflections (±0.2 degrees 2θ) at about at 6.3°, 13.8°, and 21.6°, and one or two of the 2θ-reflections (±0.2 degrees 2θ) at about 12.7°, 19.1°, and 24.5°. In some embodiments, the compound of Formula (I), mesylate salt, Form I is characterized by an XRPD pattern comprising 2θ-reflections (±0.2 degrees 2θ) at about at 6.3°, 13.8°, and 21.6°, and one of the 2θ-reflections (±0.2 degrees 2θ) at about 12.7°, 19.1°, and 24.5°. In some embodiments, the compound of Formula (I), mesylate salt, Form I is characterized by an XRPD pattern comprising 2θ-reflections (±0.2 degrees 2θ) at about at 6.3°, 13.8°, and 21.6°, and two of the 2θ-reflections (±0.2 degrees 2θ) at about 12.7°, 19.1°, and 24.5°. In some embodiments, the compound of Formula (I), mesylate salt, Form I is characterized by an XRPD pattern comprising 2θ-reflections (±0.2 degrees 2θ) at about at 6.3°, 12.7°, 13.8°, 19.1°, 21.6°, and 24.5°.

In some embodiments, the compound of Formula (I), mesylate salt, Form I is characterized by an XRPD pattern comprising 2θ-reflections (±0.2 degrees 2θ) at about at 6.3°, 12.7°, 13.8°, 19.1°, 21.6°, and 24.5°, and one, two, or three of the 2θ-reflections (±0.2 degrees 2θ) at about 14.6°, 23.4°, and 29.7°. In some embodiments, the compound of Formula (I), mesylate salt, Form I is characterized by an XRPD pattern comprising 2θ-reflections (±0.2 degrees 2θ) at about at 6.3°, 12.7°, 13.8°, 19.1°, 21.6°, and 24.5°, and one or two of the 2θ-reflections (±0.2 degrees 2θ) at about 14.6°, 23.4°, and 29.7°. In some embodiments, the compound of Formula (I), mesylate salt, Form I is characterized by an XRPD pattern comprising 2θ-reflections (±0.2 degrees 2θ) at about at 6.3°, 12.7°, 13.8°, 19.1°, 21.6°, and 24.5°, and one of the 2θ-reflections (±0.2 degrees 2θ) at about 14.6°, 23.4°, and 29.7°. In some embodiments, the compound of Formula (I), mesylate salt, Form I is characterized by an XRPD pattern comprising 2θ-reflections (±0.2 degrees 2θ) at about at 6.3°, 12.7°, 13.8°, 19.1°, 21.6°, and 24.5°, and two of the 2θ-reflections (±0.2 degrees 2θ) at about 14.6°, 23.4°, and 29.7°. In some embodiments, the compound of Formula (I), mesylate salt, Form I is characterized by an XRPD pattern comprising 2θ-reflections (±0.2 degrees 2θ) at about at 6.3°, 12.7°, 13.8°, 14.6°, 19.1°, 21.6°, 23.4°, 24.5°, and 29.7°.

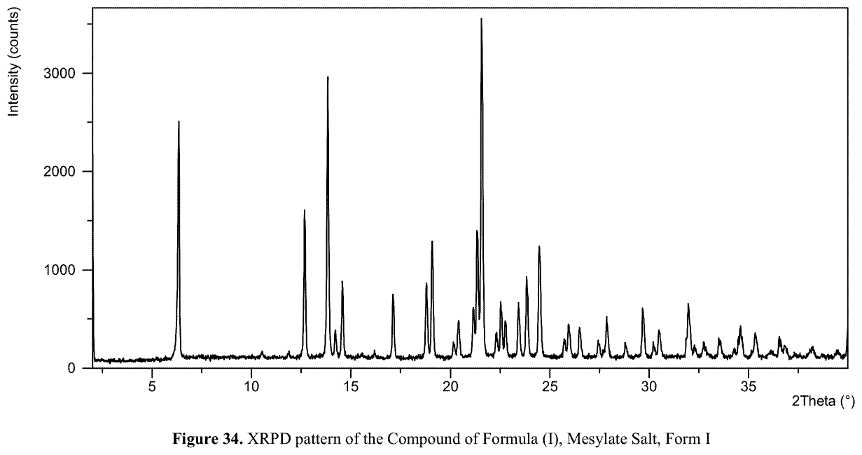

In some embodiments, the compound of Formula (I), mesylate salt, Form I is characterized by an XRPD pattern substantially as shown in FIG. 34. In some embodiments, the compound of Formula (I), mesylate salt, Form I is characterized by an XRPD pattern as listed in Table 34.

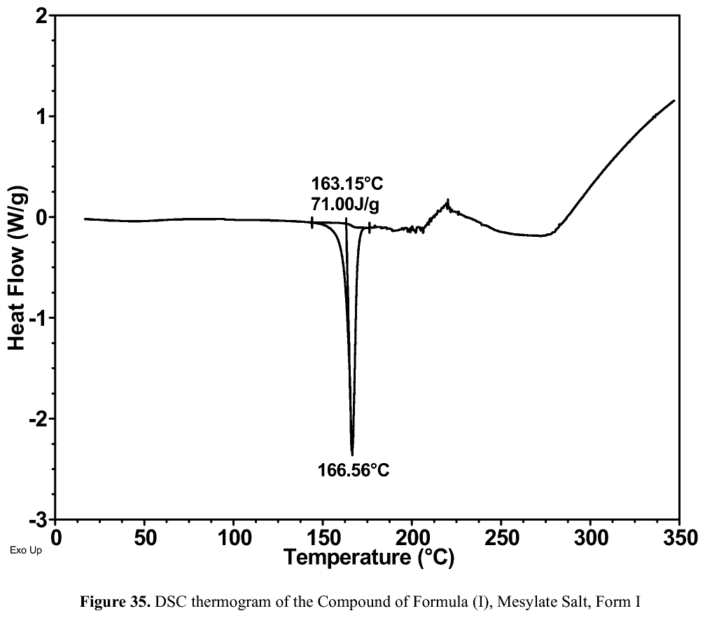

In some embodiments, the compound of Formula (I), mesylate salt, Form I is characterized by a DSC pattern comprising an endothermic transition with onset at about 163° C. In some embodiments, the compound of Formula (I), mesylate salt, Form I is characterized by a DSC pattern substantially as set forth in FIG. 35.

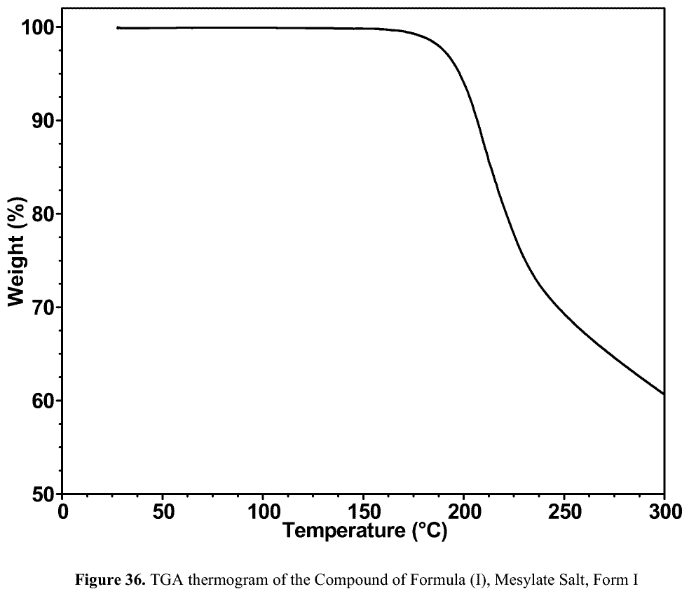

In some embodiments, the compound of Formula (I), mesylate salt, Form I is characterized by a TGA pattern substantially as set forth in FIG. 36.

### Preparation of the Crystalline Forms

In some embodiments, the present disclosure provides methods of making the crystalline forms disclosed herein.

In some embodiments, the present disclosure provides method of making the compound of Formula (I) hydrate Form H1, wherein the method comprises (i) slurrying the compound of Formula (I) in a solvent and (ii) isolating the compound of Formula (I) hydrate Form Hl. In some embodiments, isolating the compound of Formula (I) hydrate Form H1 comprises centrifugation. In some embodiments, about 10-200 mg of the compound of Formula (I) is slurried per mL of solvent. In some embodiments, about 75-150 mg of the compound of Formula (I) is slurried per mL of solvent. In some embodiments, about 25-75 mg of the compound of Formula (I) is slurried per mL of solvent. In some embodiments, the solvent comprises water, acetonitrile, or a mixture thereof. In some embodiments, the solvent comprises water and acetonitrile in a ratio of 98 to 2 (v/v). In some embodiments, the solvent comprises water and acetonitrile in a ratio of 95 to 5 (v/v).

In some embodiments, the present disclosure provides methods of making the compound of Formula (I) Form I, wherein the method comprises drying the compound of Formula (I) hydrate Form Hl. In some embodiments, the compound of Formula (I) hydrate Form H1 is dried at a temperature of about 20° C. to 100° C. In some embodiments, the compound of Formula (I) hydrate Form H1 is dried at a temperature of about 50° C. In some embodiments, the compound of Formula (I) hydrate Form H1 is dried under vacuum.

In some embodiments, the present disclosure provides methods of making a solvated crystalline form of the compound of Formula (I), wherein the method comprises (i) slurrying a compound of Formula (I) in a solvent and (ii) isolating the solvated crystalline form. In some embodiments, about 100-400 mg of the compound of Formula (I) is slurried per mL of solvent. In some embodiments, the solvent comprises methanol, ethanol, isopropanol, 1-butanol, 2-butanol (e.g. (S)-2-butanol or (R)-2-butanol), acetone, acetonitrile, ethyl acetate, butyl acetate, methyl t-butyl ether (MTBE), tetrahydrofuran, toluene, 2-butanone (methyl ethyl ketone), 2-methyl tetrahydrofuran, heptane (e.g. n-heptane), cyclohexane, cyclopentyl methyl ether, dichloromethane, N,N-dimethylacetamide, N,N-dimethylformamide, ethyleneglycol, hexane (e.g. n-hexane), propylene glycol, methyl butyl ketone, methylcyclohexane, methylisobutylketone, N-methylpyrrolidone, t-butyl alcohol, acetic acid, anisole, dimethyl sulfoxide, isobutyl acetate, methyl acetate, 2-methyl-1-propanol, ethyl ether, ethyl formate, formic acid, pentane (e.g. n-pentane), 1-pentanol, propyl acetate (e.g. isopropyl acetate), triethylamine, or a mixture thereof. In some embodiments, the solvent comprises acetonitrile.

In some embodiments, the present disclosure provides methods of making the compound of Formula (I) Form II, wherein the method comprises drying the of Formula I acetonitrile solvate Form ACN3.

### Pharmaceutical Compositions

The crystalline forms disclosed herein may be formulated with conventional carriers and excipients. For example, tablets will contain excipients, glidants, fillers, binders, and the like. Aqueous formulations are prepared in sterile form, and when intended for delivery by other than oral administration generally will be isotonic. All formulations may optionally comprise excipients such as those set forth in the “Handbook of Pharmaceutical Excipients” (1986). Pharmaceutically acceptable excipients include ascorbic acid and other antioxidants, chelating agents such as EDTA, carbohydrates such as dextran, hydroxyalkylcellulose, hydroxyalkylmethylcellulose, stearic acid and the like. In some embodiments, the formulations comprise one or more pharmaceutically acceptable excipients. The pH of the formulations ranges from about 3 to about 11, but is ordinarily about 7 to 10. In some embodiments, the pH of the formulations ranges from about 2 to about 5, but is ordinarily about 3 to 4.

While it is possible for the crystalline forms of the disclosure (“the active ingredients”) to be administered alone it may be preferable to present them as pharmaceutical formulations. The formulations, both for veterinary and for human use, of the invention comprise at least one active ingredient, as above defined, together with one or more acceptable carriers therefor and optionally other therapeutic ingredients, particularly those additional therapeutic ingredients as discussed herein. The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any appropriate method known in the art of pharmacy. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

In some embodiments, the pharmaceutical formulation is for subcutaneous, intramuscular, intravenous, oral, or inhalation administration.

In some embodiments, the crystalline forms described herein have optimized/improved pharmacokinetic properties and are amenable to oral administration. For example, the crystalline forms disclosed herein have improved bioavailability and can therefore be administered by oral administration.

In some embodiments, the formulations of the present invention are suitable for oral administration may be presented as discrete units such as capsules, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

In some embodiments, the tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent. Molded tablets may be made by molding in a suitable machine a mixture of the powdered active ingredient moistened with an inert liquid diluent. The tablets may optionally be coated or scored and optionally are formulated so as to provide slow or controlled release of the active ingredient therefrom.

For infections of the eye or other external tissues, e.g., mouth and skin, the formulations are applied as a topical ointment or cream containing the active ingredient(s) in an amount of, for example, 0.075 to 20% w/w (including active ingredient(s) in a range between 0.1% and 20% in increments of 0.1% w/w such as 0.6% w/w, 0.7% w/w, etc.), preferably 0.2 to 15% w/w and most preferably 0.5 to 10% w/w. When formulated in an ointment, the active ingredients may be employed with either a paraffinic or a water-miscible ointment base. Alternatively, the active ingredients may be formulated in a cream with an oil-in-water cream base.

If desired, the aqueous phase of the cream base may include, for example, at least 30% w/w of a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations may desirably include a compound which enhances absorption or penetration of the active ingredient through the skin or other affected areas. Examples of such dermal penetration enhancers include dimethyl sulphoxide and related analogs.

The oily phase of the emulsions of this invention may be constituted from known ingredients in a known manner. While the phase may comprise merely an emulsifier (otherwise known as an emulgent), it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations.

Emulgents and emulsion stabilizers suitable for use in the formulation of the invention include Tween® 60, Span® 80, cetostearyl alcohol, benzyl alcohol, myristyl alcohol, glyceryl mono-stearate and sodium lauryl sulfate. Further emulgents and emulsion stabilizers suitable for use in the formulation of the invention include Tween® 80.

The choice of suitable oils or fats for the formulation is based on achieving the desired cosmetic properties. The cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils are used.

Pharmaceutical formulations according to the present invention comprise a crystalline form disclosed herein together with one or more pharmaceutically acceptable carriers or excipients and optionally other therapeutic agents. Pharmaceutical formulations containing the active ingredient may be in any form suitable for the intended method of administration. When used for oral use for example, tablets, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcelluose, sodium alginate, polyvinylpyrrolidone, gum tragacanth and gum acacia, and dispersing or wetting agents such as a naturally-occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin. Further non-limiting examples of suspending agents include Cyclodextrin. In some examples, the suspending agent is Sulfobutyl ether beta-cyclodextrin (SEB-beta-CD), for example Captisol®.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth above, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions of the invention may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally-occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables. Among the acceptable vehicles and solvents that may be employed are water, Ringer's solution isotonic sodium chloride solution, and hypertonic sodium chloride solution.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 mg of the active ingredient per milliliter of solution in order that infusion of a suitable volume at a rate of about mL/hr can occur.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of 0.5 to 20%, advantageously 0.5 to 10%, and particularly about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration may be presented as a suppository with a suitable base comprising for example cocoa butter or a salicylate.

In some embodiments, the crystalline forms disclosed herein are administered by inhalation. In some embodiments, formulations suitable for intrapulmonary or nasal administration have a particle size for example in the range of 0.1 to 500 microns, such as 0.5, 1, 35 etc., which is administered by rapid inhalation through the nasal passage or by inhalation through the mouth so as to reach the alveolar sacs. Suitable formulations include aqueous or oily solutions of the active ingredient. Formulations suitable for aerosol or dry powder administration may be prepared according to conventional methods and may be delivered with other therapeutic agents. In some embodiments, the crystalline forms used herein are formulated and dosed as dry powder. In some embodiments, the crystalline forms used herein are formulated and dosed as a nebulized formulation. In some embodiments, the crystalline forms used herein are formulated for delivery by a face mask. In some embodiments, the crystalline forms used herein are formulated for delivery by a face tent.

Formulations suitable for vaginal administration may be presented as pessaries, tampons, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The invention further provides veterinary compositions comprising at least one active ingredient as above defined together with a veterinary carrier therefor.

Veterinary carriers are materials useful for the purpose of administering the composition and may be solid, liquid or gaseous materials which are otherwise inert or acceptable in the veterinary art and are compatible with the active ingredient. These veterinary compositions may be administered orally, parenterally or by any other desired route.

Crystalline forms of the invention are used to provide controlled release pharmaceutical formulations containing as active ingredient one or more crystalline forms of the invention (“controlled release formulations”) in which the release of the active ingredient are controlled and regulated to allow less frequency dosing or to improve the pharmacokinetic or toxicity profile of a given active ingredient.

### Kits

Also provided herein are kits that includes a crystalline form disclosed herein. In some embodiments the kits described herein may comprise a label and/or instructions for use of the crystalline form in the treatment of a disease or condition in a subject (e.g., human) in need thereof. In some embodiments, the disease or condition is viral infection.

In some embodiments, the kit may also comprise one or more additional therapeutic agents and/or instructions for use of additional therapeutic agents in combination with the crystalline form disclosed herein in the treatment of the disease or condition in a subject (e.g., human) in need thereof.

In some embodiments, the kits provided herein comprises individual dose units of a crystalline form as described herein. Examples of individual dosage units may include pills, tablets, capsules, prefilled syringes or syringe cartridges, IV bags, inhalers, nebulizers etc., each comprising a therapeutically effective amount of the crystalline form in question. In some embodiments, the kit may contain a single dosage unit and in others, multiple dosage units are present, such as the number of dosage units required for a specified regimen or period.

Also provided are articles of manufacture that include a crystalline form disclosed herein and a container. In some embodiments, the container of the article of manufacture is a vial, jar, ampoule, preloaded syringe, blister package, tin, can, bottle, box, an intravenous bag, an inhaler, or a nebulizer.

### Administration

One or more crystalline forms of the disclosure are administered by any route appropriate to the condition to be treated. Suitable routes include oral, rectal, inhalation, pulmonary, topical (including buccal and sublingual), vaginal and parenteral (including subcutaneous, intramuscular, intravenous, intradermal, intrathecal and epidural), and the like. In some embodiments, crystalline form disclosed herein are administered by inhalation or intravenously. In some embodiments, the crystalline form disclosed herein are administered orally. It will be appreciated that the preferred route may vary with for example the condition of the recipient.

In the methods of the present invention for the treatment of a viral infection, the crystalline form disclosed herein can be administered at any time to a human who may come into contact with the virus or is already suffering from the viral infection. In some embodiments, the crystalline form disclosed herein can be administered prophylactically to humans coming into contact with humans suffering from the viral infection or at risk of coming into contact with humans suffering from the viral infection, e.g., healthcare providers. In some embodiments, administration of the crystalline form disclosed herein can be to humans testing positive for the viral infection but not yet showing symptoms of the viral infection. In some embodiments, administration of the crystalline form disclosed herein can be to humans upon commencement of symptoms of the viral infection.

In some embodiments, the methods disclosed herein comprise event driven administration of the crystalline form disclosed herein to the subject.

As used herein, the terms “event driven” or “event driven administration” refer to administration of the crystalline form described herein, (1) prior to an event (e.g., 2 hours, 1 day, 2 days, 5 day, or 7 or more days prior to the event) that would expose the individual to the virus (or that would otherwise increase the individual's risk of acquiring the viral infection); and/or (2) during an event (or more than one recurring event) that would expose the individual to the virus (or that would otherwise increase the individual's risk of acquiring the viral infection); and/or (3) after an event (or after the final event in a series of recurring events) that would expose the individual to the virus (or that would otherwise increase the individual's risk of acquiring the viral infection). In some embodiments, the event driven administration is performed pre-exposure of the subject to the virus. In some embodiments, the event driven administration is performed post-exposure of the subject to the virus. In some embodiments, the event driven administration is performed pre-exposure of the subject to the virus and post-exposure of the subject to the virus.

In certain embodiments, the methods disclosed herein involve administration prior to and/or after an event that would expose the individual to the virus or that would otherwise increase the individual's risk of acquiring the viral infection, e.g., as pre-exposure prophylaxis (PrEP) and/or as post-exposure prophylaxis (PEP). In some embodiments, the methods disclosed herein comprise pre-exposure prophylaxis (PrEP). In some embodiments, methods disclosed herein comprise post-exposure prophylaxis (PEP).

In some embodiments, a crystalline form disclosed herein is administered before exposure of the subject to the virus.

In some embodiments, a crystalline form disclosed herein is administered before and after exposure of the subject to the virus.

In some embodiments, a crystalline form disclosed herein is administered after exposure of the subject to the virus.

An example of event driven dosing regimen includes administration a crystalline form disclosed herein within 24 to 2 hours prior to the virus, followed by administration of a crystalline form disclosed herein every 24 hours during the period of exposure, followed by a further administration of a crystalline form disclosed herein after the last exposure, and one last administration of a crystalline form disclosed herein 24 hours later.

A further example of an event driven dosing regimen includes administration of a crystalline form disclosed herein within 24 hours before the viral exposure, then daily administration during the period of exposure, followed by a last administration approximately 24 hours later after the last exposure (which may be an increased dose, such as a double dose).

The specific dose level of a crystalline form disclosed herein for any particular subject will depend upon a variety of factors including the activity of the specific crystalline form employed, the age, body weight, general health, sex, diet, time of administration, route of administration, and rate of excretion, drug combination and the severity of the particular disease in the subject undergoing therapy. For example, a dosage may be expressed as a number of milligrams of a crystalline form disclosed herein per kilogram of the subject's body weight (mg/kg). Dosages of between about 0.1 and 150 mg/kg may be appropriate. In some embodiments, about 0.1 and 100 mg/kg may be appropriate. In other embodiments a dosage of between 0.5 and 60 mg/kg may be appropriate. Normalizing according to the subject's body weight is particularly useful when adjusting dosages between subjects of widely disparate size, such as occurs when using the drug in both children and adult humans or when converting an effective dosage in a non-human subject such as dog to a dosage suitable for a human subject.

The daily dosage may also be described as a total amount of a crystalline form disclosed herein administered per dose or per day. Daily dosage of a crystalline form disclosed herein may be between about 1 mg and 4,000 mg, between about 2,000 to 4,000 mg/day, between about 1 to 2,000 mg/day, between about 1 to 1,000 mg/day, between about 10 to 500 mg/day, between about 20 to 500 mg/day, between about 50 to 300 mg/day, between about 75 to 200 mg/day, or between about 15 to 150 mg/day.

The dosage or dosing frequency of a crystalline form disclosed herein may be adjusted over the course of the treatment, based on the judgment of the administering physician.

The crystalline forms disclosed herein may be administered to an individual (e.g., a human) in a therapeutically effective amount. In some embodiments, the crystalline forms are administered once daily.

The crystalline forms disclosed herein can be administered by any useful route and means, such as by oral or parenteral (e.g., intravenous) administration. Therapeutically effective amounts of the crystalline forms may include from about 0.00001 mg/kg body weight per day to about 10 mg/kg body weight per day, such as from about 0.0001 mg/kg body weight per day to about 10 mg/kg body weight per day, or such as from about 0.001 mg/kg body weight per day to about 1 mg/kg body weight per day, or such as from about 0.01 mg/kg body weight per day to about 1 mg/kg body weight per day, or such as from about 0.05 mg/kg body weight per day to about 0.5 mg/kg body weight per day. In some embodiments, a therapeutically effective amount of the crystalline forms provided herein include from about 0.3 mg to about 30 mg per day, or from about 30 mg to about 300 mg per day, or from about 0.3 mg to about 30 mg per day, or from about 30 mg to about 300 mg per day.

A crystalline forms of the present disclosure may be combined with one or more additional therapeutic agents in any dosage amount of the crystalline form of the present disclosure (e.g., from 1 mg to 1000 mg of crystalline form). Therapeutically effective amounts may include from about 0.1 mg per dose to about 1000 mg per dose, such as from about 50 mg per dose to about 500 mg per dose, or such as from about 100 mg per dose to about 400 mg per dose, or such as from about 150 mg per dose to about 350 mg per dose, or such as from about 200 mg per dose to about 300 mg per dose, or such as from about 0.01 mg per dose to about 1000 mg per dose, or such as from about 0.01 mg per dose to about 100 mg per dose, or such as from about 0.1 mg per dose to about 100 mg per dose, or such as from about 1 mg per dose to about 100 mg per dose, or such as from about 1 mg per dose to about 10 mg per dose, or such as from about 1 mg per dose to about 1000 mg per dose. Other therapeutically effective amounts of the crystalline forms are about 1 mg per dose, or about 2, 3, 4, 5, 6, 7, 8, 9, 10, 15, 20, 25, 30, 40, 45, 50, 55, 60, 65, 70, 75, 80, 85, 90, 95, or about 100 mg per dose. Other therapeutically effective amounts of the crystalline forms of the present disclosure are about 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, or about 1000 mg per dose.

In some embodiments, the methods described herein comprise administering to the subject an initial daily dose of about 1 to 500 mg of a crystalline form provided herein and increasing the dose by increments until clinical efficacy is achieved. Increments of about 5, 10, 50, or 100 mg can be used to increase the dose. The dosage can be increased daily, every other day, twice per week, once per week, once every two weeks, once every three weeks, or once a month.

When administered orally, the total daily dosage for a human subject may be between about 1-4,000 mg/day, between about 1-3,000 mg/day, between 1-2,000 mg/day, about 1-1,000 mg/day, between about 10-500 mg/day, between about 50-300 mg/day, between about mg/day, or between about 100-150 mg/day. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000 mg/day administered in a single dose. In some embodiments, the total daily dosage for a human subject may be about 200, 300, 400, 500, 600, 700, or 800 mg/day administered in a single dose. In some embodiments, the total daily dosage for a human subject may be about 300, 400, 500, or 600 mg/day administered in a single dose. In some embodiments, the total daily dosage for a human subject may be about 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, or 4000 mg/day. In some embodiments, the total daily dosage for a human subject may be about 100-200, 100-300, 100-400, 100-500, 100-600, 100-700, 100-800, 100-900, 100-1000, 500-1100, 500-1200, 500-1300, 500-1400, 500-1500, 500-1600, 500-1700, 500-1800, 500-1900, 500-2000, 1500-2100, 1500-2200, 1500-2300, 1500-2400, 1500-2500, 2000-2600, 2000-2700, 2000-2800, 2000-2900, 2000-3000, 2500-3100, 2500-3200, 2500-3300, 2500-3400, 2500-3500, 3000-3600, 3000-3700, 3000-3800, 3000-3900, or 3000-4000 mg/day. In some embodiments, the total daily dosage for a human subject is about 500-1000 mg/day administered once or twice daily.

In some embodiments, the total daily dosage for a human subject may be about 100 mg/day administered in a single dose. In some embodiments, the total daily dosage for a human subject may be about 150 mg/day administered in a single dose. In some embodiments, the total daily dosage for a human subject may be about 200 mg/day administered in a single dose. In some embodiments, the total daily dosage for a human subject may be about 250 mg/day administered in a single dose. In some embodiments, the total daily dosage for a human subject may be about 300 mg/day administered in a single dose. In some embodiments, the total daily dosage for a human subject may be about 350 mg/day administered in a single dose. In some embodiments, the total daily dosage for a human subject may be about 400 mg/day administered in a single dose. In some embodiments, the total daily dosage for a human subject may be about 450 mg/day administered in a single dose. In some embodiments, the total daily dosage for a human subject may be about 500 mg/day administered in a single dose. In some embodiments, the total daily dosage for a human subject may be about 550 mg/day administered in a single dose. In some embodiments, the total daily dosage for a human subject may be about 600 mg/day administered in a single dose. In some embodiments, the total daily dosage for a human subject may be about 650 mg/day administered in a single dose. In some embodiments, the total daily dosage for a human subject may be about 700 mg/day administered in a single dose. In some embodiments, the total daily dosage for a human subject may be about 750 mg/day administered in a single dose. In some embodiments, the total daily dosage for a human subject may be about 800 mg/day administered in a single dose. In some embodiments, the total daily dosage for a human subject may be about 850 mg/day administered in a single dose. In some embodiments, the total daily dosage for a human subject may be about 900 mg/day administered in a single dose. In some embodiments, the total daily dosage for a human subject may be about 950 mg/day administered in a single dose. In some embodiments, the total daily dosage for a human subject may be about 1000 mg/day administered in a single dose. In some embodiments, the total daily dosage for a human subject may be about 1500 mg/day administered in a single dose. In some embodiments, the total daily dosage for a human subject may be about 2000 mg/day administered in a single dose. In some embodiments, the total daily dosage for a human subject may be about 2500 mg/day administered in a single dose. In some embodiments, the total daily dosage for a human subject may be about 3000 mg/day administered in a single dose. In some embodiments, the total daily dosage for a human subject may be about 4000 mg/day administered in a single dose.

In some embodiments, the total daily dosage for a human subject may be about 100 mg/day administered in two dose daily. In some embodiments, the total daily dosage for a human subject may be about 150 mg/day administered in two dose daily. In some embodiments, the total daily dosage for a human subject may be about 200 mg/day administered in two dose daily. In some embodiments, the total daily dosage for a human subject may be about 250 mg/day administered in two dose daily. In some embodiments, the total daily dosage for a human subject may be about 300 mg/day administered in two dose daily. In some embodiments, the total daily dosage for a human subject may be about 350 mg/day administered in two dose daily. In some embodiments, the total daily dosage for a human subject may be about 400 mg/day administered in two dose daily. In some embodiments, the total daily dosage for a human subject may be about 450 mg/day administered in two dose daily. In some embodiments, the total daily dosage for a human subject may be about 500 mg/day administered in two dose daily. In some embodiments, the total daily dosage for a human subject may be about 550 mg/day administered in two dose daily. In some embodiments, the total daily dosage for a human subject may be about 600 mg/day administered in two dose daily. In some embodiments, the total daily dosage for a human subject may be about 650 mg/day administered in two dose daily. In some embodiments, the total daily dosage for a human subject may be about 700 mg/day administered in two dose daily. In some embodiments, the total daily dosage for a human subject may be about 750 mg/day administered in two dose daily. In some embodiments, the total daily dosage for a human subject may be about 800 mg/day administered in two dose daily. In some embodiments, the total daily dosage for a human subject may be about 850 mg/day administered in two dose daily. In some embodiments, the total daily dosage for a human subject may be about 900 mg/day administered in two dose daily. In some embodiments, the total daily dosage for a human subject may be about 950 mg/day administered in two dose daily e. In some embodiments, the total daily dosage for a human subject may be about 1000 mg/day administered two dose daily. In some embodiments, the total daily dosage for a human subject may be about 1500 mg/day administered two dose daily. In some embodiments, the total daily dosage for a human subject may be about 2000 mg/day administered two dose daily. In some embodiments, the total daily dosage for a human subject may be about 2500 mg/day administered two dose daily. In some embodiments, the total daily dosage for a human subject may be about 3000 mg/day administered two dose daily. In some embodiments, the total daily dosage for a human subject may be about 4000 mg/day administered two dose daily.

A single dose can be administered hourly, daily, weekly, or monthly. For example, a single dose can be administered once every 1 hour, 2, 3, 4, 6, 8, 12, 16 or once every 24 hours. A single dose can also be administered once every 1 day, 2, 3, 4, 5, 6, or once every 7 days. A single dose can also be administered once every 1 week, 2, 3, or once every 4 weeks. In certain embodiments, a single dose can be administered once every week. A single dose can also be administered once every month. In some embodiments, a crystalline form disclosed herein is administered once daily in a method disclosed herein. In some embodiments, a crystalline form disclosed herein is administered twice daily in a method disclosed herein. In some embodiments, a crystalline form disclosed herein is administered three times daily in a method disclosed herein.

In some embodiments, a crystalline form disclosed herein is administered once daily in the total daily dose of 100-4000 mg/day. In some embodiments, a crystalline form disclosed herein is administered twice daily in the total daily dose of 100-4000 mg/day. In some embodiments, a crystalline form disclosed herein is administered three times daily in the total daily dose of 100-4000 mg/day. In some embodiments, a crystalline form disclosed herein is administered once daily in the total daily dose of 300-900 mg/day.

The frequency of dosage of the crystalline form of the present disclosure will be determined by the needs of the individual patient and can be, for example, once per day or twice, or more times, per day. Administration of the crystalline form continues for as long as necessary to treat the viral infection. For example, a crystalline form can be administered to a human being infected with the virus for a period of from 20 days to 180 days or, for example, for a period of from 20 days to 90 days or, for example, for a period of from 30 days to 60 days.

Administration can be intermittent, with a period of several or more days during which a patient receives a daily dose of a crystalline form of the present disclosure followed by a period of several or more days during which a patient does not receive a daily dose of the crystalline form. For example, a patient can receive a dose of the crystalline form every other day, or three times per week. Again by way of example, a patient can receive a dose of the crystalline form each day for a period of from 1 to 14 days, followed by a period of 7 to 21 days during which the patient does not receive a dose of the crystalline form, followed by a subsequent period (e.g., from 1 to 14 days) during which the patient again receives a daily dose of the crystalline form. Alternating periods of administration of the crystalline form, followed by non-administration of the crystalline form, can be repeated as clinically required to treat the patient.

The crystalline forms of the present disclosure or the pharmaceutical compositions thereof may be administered once, twice, three, or four times daily, using any suitable mode described above. Also, administration or treatment with the crystalline forms may be continued for a number of days; for example, commonly treatment would continue for at least 3 days, at least 5 days, at least 7 days, 14 days, or 28 days, for one cycle of treatment.

### Methods of Use

The present disclosure also provides a method of treating or preventing a viral infection in a subject (e.g., human) in need thereof, the method comprising administering to the subject a crystalline form described herein.

In some embodiments, the present disclosure provides a method of treating a viral infection in a subject (e.g., human) in need thereof, the method comprising administering to a subject in need thereof a crystalline form described herein.

In some embodiments, the crystalline form described herein is administered to the human via oral, intramuscular, intravenous, subcutaneous, or inhalation administration.

In some embodiments, the present disclosure provides for methods of treating or preventing a viral infection in a subject (e.g., human) in need thereof, the method comprising administering to the subject a crystalline form disclosed herein and at least one additional active therapeutic or prophylactic agent.

In some embodiments, the present disclosure provides for methods of treating a viral infection in a subject (e.g., human) in need thereof, the method comprising administering to the subject a crystalline form disclosed herein, and at least one additional active therapeutic or prophylactic agent.

In one embodiment, the present disclosure provides for methods of inhibiting a viral polymerase in a cell, the methods comprising contacting the cell infected by a virus with a crystalline form disclosed herein, whereby the viral polymerase is inhibited.

In one embodiment, the present disclosure provides for methods of inhibiting a viral polymerase in a cell, the methods comprising contacting the cell infected by a virus with a crystalline form disclosed herein, and at least one additional active therapeutic agent, whereby the viral polymerase is inhibited.

Also provided here are the uses of the crystalline forms disclosed herein for use in treating or preventing a viral infection in a subject in need thereof. For example, provided herein are uses of the crystalline forms disclosed herein for use in treating a viral infection in a subject in need thereof.

In some embodiments, the viral infection is a paramyxoviridae virus infection. As such, in some embodiments, the present disclosure provides methods for treating a paramyxoviridae infection in a subject (e.g., a human) in need thereof, the method comprising administering to the subject a crystalline form disclosed herein. Paramyxoviridae viruses include, but are not limited to Nipah virus, Hendra virus, measles, mumps, and parainfluenze virus.

In some embodiments, the viral infection is a human parainfluenza virus, Nipah virus, Hendra virus, measles, or mumps infection.

In some embodiments, the viral infection is a pneumoviridae virus infection. As such, in some embodiments, the present disclosure provides a method of treating a pneumoviridae virus infection in a human in need thereof, the method comprising administering to the human a crystalline form provided herein. Pneumoviridae viruses include, but are not limited to, respiratory snycytial virus and human metapneumovirus. In some embodiments, the pneumoviridae virus infection is a respiratory syncytial virus infection. In some embodiments, the pneumoviridae virus infection is human metapneumovirus infection.

In some embodiments, the present disclosure provides a crystalline form disclosed herein, for use in the treatment of a pneumoviridae virus infection in a human in need thereof. In some embodiments, the pneumoviridae virus infection is a respiratory syncytial virus infection. In some embodiments, the pneumoviridae virus infection is human metapneumovirus infection.

In some embodiments, the present disclosure provides methods for treating a RSV infection in a human in need thereof, the method comprising administering to the human a crystalline form provided herein. In some embodiments, the human is suffering from a chronic respiratory syncytial viral infection. In some embodiments, the human is acutely infected with RSV.

In some embodiments, a method of inhibiting RSV replication is provided, wherein the method comprises administering to a human in need thereof, a crystalline form disclosed herein, wherein the administration is by inhalation.

In some embodiments, the present disclosure provides a method for reducing the viral load associated with RSV infection, wherein the method comprises administering to a human infected with RSV a crystalline form disclosed herein.

In some embodiments, the viral infection is a picornaviridae virus infection. As such, in some embodiments, the present disclosure provides a method of treating a picornaviridae virus infection in a human in need thereof, the method comprising administering to the human a crystalline form of the present disclosure. Picornaviridae viruses are eneteroviruses causing a heterogeneous group of infections including herpangina, aseptic meningitis, a common-cold-like syndrome (human rhinovirus infection), a non-paralytic poliomyelitis-like syndrome, epidemic pleurodynia (an acute, febrile, infectious disease generally occurring in epidemics), hand-foot-mouth syndrome, pediatric and adult pancreatitis and serious myocarditis. In some embodiments, the Picornaviridae virus infection is human rhinovirus infection (HRV). In some embodiments, the Picornaviridae virus infection is HRV-A, HRV-B, or HRV-C infection.

In some embodiments, the viral infection is selected from the group consisting of Coxsackie A virus infection, Coxsackie A virus infection, enterovirus D68 infection, enterovirus B69 infection, enterovirus D70 infection, enterovirus A71 infection, and poliovirus infection.

In some embodiments, the present disclosure provides a crystalline form, for use in the treatment of a picornaviridae virus infection in a human in need thereof. In some embodiments, the picornaviridae virus infection is human rhinovirus infection.

In some embodiments, the viral infection is a flaviviridae virus infection. As such, in some embodiments, the present disclosure provides a method of treating a flaviviridae virus infection in a human in need thereof, the method comprising administering to the human a crystalline form described herein. Representative flaviviridae viruses include, but are not limited to, dengue, Yellow fever, West Nile, Zika, Japanese encephalitis virus, and Hepatitis C (HCV). In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a Japanese ensephalitis virus infection. In some embodiments, the flaviviridae virus infection is a hepatitis C virus infection.

In some embodiments, the flaviviridae virus infection is a dengue virus infection, yellow fever virus infection, West Nile virus infection, tick borne encephalitis, Kunjin Japanese encephalitis, St. Louis encephalitis, Murray valley encephalitis, Omsk hemorrhagic fever, bovine viral diarrhea, zika virus infection, or a HCV infection.

In some embodiments, the present disclosure provides use of a crystalline form disclosed herein for treatment of a flaviviridae virus infection in a human in need thereof. In some embodiments, the flaviviridae virus infection is a dengue virus infection. In some embodiments, the flaviviridae virus infection is a yellow fever virus infection. In some embodiments, the flaviviridae virus infection is a West Nile virus infection. In some embodiments, the flaviviridae virus infection is a zika virus infection. In some embodiments, the flaviviridae virus infection is a hepatitis C virus infection.

In some embodiments, the viral infection is a filoviridae virus infection. As such, in some embodiments, provided herein is a method of treating a filoviridae virus infection in a human in need thereof, the method comprising administering to the human a crystalline form disclosed herein. Representative filoviridae viruses include, but are not limited to, ebola (variants Zaire, Bundibugio, Sudan, Tai forest, or Reston) and marburg. In some embodiments, the filoviridae virus infection is an ebola virus infection. In some embodiments, the filoviridae virus infection is a marburg virus infection.

In some embodiments, the present disclosure provides a crystalline form for use in the treatment of a filoviridae virus infection in a human in need thereof. In some embodiments, the filoviridae virus infection is an ebola virus infection. In some embodiments, the filoviridae virus infection is a marburg virus infection.

In some embodiments, the viral infection is a coronavirus infection. As such, in some embodiments, provided herein is a method of treating a coronavirus infection in a human in need thereof, wherein the method comprises administering to the human a crystalline form provided herein. In some embodiments, the coronavirus infection is a Severe Acute Respiratory Syndrome (SARS-CoV) infection, Middle Eastern Respiratory Syndrome (MERS) infection, SARS-CoV-2 infection, other human coronavirus (229E, NL63, OC43, HKU1, or WIV1) infections, zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection. In some embodiments, the viral infection is a zoonotic coronavirus infection, In some embodiments, the viral infection is caused by a virus having at least 70% sequence homology to a viral polymerase selected from the group consisting of SARS-CoV polymerase, MERS-CoV polymerase and SARS-CoV-2. In some embodiments, the viral infection is caused by a virus having at least 80% sequence homology to a viral polymerase selected from the group consisting of SARS-CoV polymerase, MERS-CoV polymerase and SARS-CoV-2. In some embodiments, the viral infection is caused by a virus having at least 90% sequence homology to a viral polymerase selected from the group consisting of SARS-CoV polymerase, MERS-CoV polymerase and SARS-CoV-2. In some embodiments, the viral infection is caused by a virus having at least 95% sequence homology to a viral polymerase selected from the group consisting of SARS-CoV polymerase, MERS-CoV polymerase and SARS-CoV-2.

In some embodiments, the viral infection is caused by a variant of SARS-CoV-2, for example by the B.1.1.7 variant (the UK variant), B.1.351 variant (the South African variant), P.1 variant (the Brazil variant), B.1.1.7 with E484K variant, B.1.1.207 variant, B.1.1.317 variant, B.1.1.318 variant, B.1.429 variant, B.1.525 variant, or P.3 variant. In some embodiments, the viral infection is caused by the B.1.1.7 variant of SARS-CoV-2. In some embodiments, the viral infection is caused by the B.1.351 variant of SARS-CoV-2. In some embodiments, the viral infection is caused by the P.1 variant of SARS-CoV-2.

In some embodiments, the present disclosure provides a crystalline form for use in the treatment of a coronavirus virus infection in a human in need thereof. In some embodiments, the coronavirus infection is a Severe Acute Respiratory Syndrome (SARS) infection, Middle Eastern Respiratory Syndrome (MERS) infection, SARS-CoV-2 infection, other human coronavirus (229E, NL63, 0C43, HKU1, or WIV1) infections, and zoonotic coronavirus (PEDV or HKU CoV isolates such as HKU3, HKU5, or HKU9) infections. In some embodiments, the viral infection is a Severe Acute Respiratory Syndrome (SARS) infection. In some embodiments, the viral infection is a Middle Eastern Respiratory Syndrome (MERS) infection. In some embodiments, the viral infection is SARS-CoV-2 infection (COVID19).

In some embodiments, the viral infection is an arenaviridae virus infection. As such, in some embodiments, the disclosure provides a method of treating an arenaviridae virus infection in a human in need thereof, the method comprising administering to the human a crystalline form disclosed herein. In some embodiments, the arenaviridae virus infection is a Lassa infection or a Junin infection.

In some embodiments, the present disclosure provides a crystalline form for use in the treatment of an arenaviridae virus infection in a human in need thereof. In some embodiments, the arenaviridae virus infection is a Lassa infection or a Junin infection.

In some embodiments, the viral infection is an orthomyxovirus infection, for example, an influenza virus infection. In some embodiments, the viral infection is an influenza virus A, influenza virus B, or influenza virus C infection.

As described more fully herein, the crystalline forms described herein can be administered with one or more additional therapeutic agent(s) to an individual (e.g., a human) infected with a viral infection. The additional therapeutic agent(s) can be administered to the infected individual at the same time as the compound of the present disclosure or before or after administration of the compound of the present disclosure.

### Combination Therapy

The crystalline forms described herein can also be used in combination with one or more additional therapeutic agents. As such, also provided herein are methods of treatment of a viral infection in a subject in need thereof, wherein the methods comprise administering to a subject in need thereof a crystalline form of the disclosure and a therapeutically effective amount of one or more additional therapeutic agents.

In some embodiments, the additional therapeutic agent is an antiviral agent. Any suitable antiviral agent can be used in the methods described herein. In some embodiments, the antiviral agent is selected from the group consisting of 5-substituted 2′-deoxyuridine analogues, Cytochrome P450 3A4 inhibitors, Peptidyl-prolyl cis-trans isomerase A inhibitors, nucleoside analogues, pyrophosphate analogues, nucleoside reverse transcriptase inhibitors, non-nucleoside reverse transcriptase inhibitors, protease inhibitors, integrase inhibitors, entry inhibitors, acyclic guanosine analogues, acyclic nucleoside phosphonate analogues, HCV NS5A/NS5B inhibitors, influenza virus inhibitors, interferons, immunostimulators, oligonucleotides, antimitotic inhibitors, and combinations thereof.

In some embodiments, the additional therapeutic agent is a 5-substituted 2′-deoxyuridine analogue. For example, in some embodiments, the additional therapeutic agent is selected from the group consisting of idoxuridine, trifluridine, brivudine [BVDU], and combinations thereof.

In some embodiments, the additional therapeutic agent is a nucleoside analogue. For example, in some embodiments, the additional therapeutic agent is selected from the group consisting of vidarabine, entecavir (ETV), telbivudine, lamivudine, adefovir dipivoxil, tenofovir disoproxil fumarate (TDF) and combinations thereof. In some embodiments, the additional therapeutic agent is favipiravir, ribavirin, galidesivir, or a combination thereof. In some embodiments, the additional therapeutic agent is β-D-N4-hydroxycytidine.

In some embodiments, the additional therapeutic agent is a pyrophosphate analogue. For example, in some embodiments, the additional therapeutic agent is foscarnet or phosphonoacetic acid.

In some embodiments, the additional therapeutic agent is nucleoside reverse transcriptase inhibitor. In some embodiments, the antiviral agent is zidovudine, didanosine zalcitabine, stavudine, lamivudine, abacavir, emtricitabine, and combinations thereof. In some embodiments, the additional therapeutic agent is sangivamycin, β-d-N4-Hydroxycytidine (NHC), MK-4482 (EIDD-2801), EIDD-1931, or a combination thereof.

In some embodiments, the additional therapeutic agent is a non-nucleoside reverse transcriptase inhibitor. In some embodiments, the antiviral agent is selected from the group consisting of nevirapine, delavirdine, efavirenz, etravirine, rilpivirine, and combinations thereof.

In some embodiments, the additional therapeutic agent is a protease inhibitor. In some embodiments, the protease inhibitor is a HIV protease inhibitor. For example, in some embodiments, the antiviral agent is selected from the group consisting of saquinavir, ritonavir, indinavir, nelfinavir, amprenavir, lopinavir, atazanavir, fosamprenavir, darunavir, tipranavir, cobicistat, and combinations thereof. In some embodiments, the protease inhibitor is a HCV NS3/4A protease inhibitor. For example, in some embodiments, the additional therapeutic agent is selected from the group consisting of voxilaprevir, asunaprevir, boceprevir, paritaprevir, simeprevir, telaprevir, vaniprevir, grazoprevir, ribavirin, danoprevir, faldaprevir, vedroprevir, sovaprevir, deldeprevir, narlaprevir and combinations thereof. In some embodiments, the additional therapeutic agent is selected from the group consisting of voxilaprevir, asunaprevir, boceprevir, paritaprevir, simeprevir, telaprevir, vaniprevir, grazoprevir, and combinations thereof.

In some embodiments, the additional therapeutic agent is an integrase inhibitor. For example, in some embodiments, the additional therapeutic agent is selected from the group consisting of raltegravir, dolutegravir, elvitegravir, abacavir, lamivudine, and combinations thereof. In some embodiments, the additional therapeutic agent is selected from the group consisting of bictegravir, raltegravir, dolutegravir, cabotegravir, elvitegravir, and combinations thereof. In some embodiments, the additional therapeutic agent is selected from the group consisting of bictegravir, dolutegravir, and cabotegravir, and combinations thereof. In some embodiments, the additional therapeutic agent is bictegravir.

In some embodiments, the additional therapeutic agent is an entry inhibitor. For example, in some embodiments, the additional therapeutic agent is selected from the group consisting of docosanol, enfuvirtide, maraviroc, ibalizumab, fostemsavir, leronlimab, palivizumab, respiratory syncytial virus immune globulin, intravenous [RSV-IGIV], varicella-zoster immunoglobulin [VariZIG], varicella-zoster immune globulin [VZIG]), and combinations thereof.

In some embodiments, the additional therapeutic agent is an acyclic guanosine analogue. For example, in some embodiments, the additional therapeutic agent is selected from the group consisting of acyclovir, ganciclovir, valacyclovir (also known as valaciclovir), valganciclovir, penciclovir, famciclovir, and combinations thereof.

In some embodiments, the additional therapeutic agent is an acyclic nucleoside phosphonate analogues. For example, in some embodiments, the additional therapeutic agent is selected from a group consisting of cidofovir, adefovir, adefovir dipivoxil, tenofovir, TDF, emtricitabine, efavirenz, rilpivirine, elvitegravir, and combinations thereof. In some embodiment, the additional therapeutic agent is selected from the group consisting of cidofovir, adefovir, adefovir dipivoxil, tenofovir, TDF, and combinations thereof. In some embodiment, the additional therapeutic agent is selected from the group consisting of cidofovir, adefovir dipivoxil, TDF, and combinations thereof.

In some embodiments, the additional therapeutic agent is a HCV NS5A/NS5B inhibitor. In some embodiments, the additional therapeutic agent is a NS3/4A protease inhibitor. In some embodiments, the additional therapeutic agent is a NS5A protein inhibitor. In some embodiments, the additional therapeutic agent is a NS5B polymerase inhibitor of the nucleoside/nucleotide type. In some embodiments, the additional therapeutic agent is a NS5B polymerase inhibitor of the nonnucleoside type. In some embodiments, the additional therapeutic agent is selected from the group consisting of daclatasvir, ledipasvir, velpatasvir, ombitasvir, elbasvir, sofosbuvir, dasabuvir, ribavirin, asunaprevir, simeprevir, paritaprevir, ritonavir, elbasvir, grazoprevir, and combinations thereof. In some embodiments, the additional therapeutic agent is selected from the group consisting of AT-527, daclatasvir, ledipasvir, velpatasvir, ombitasvir, elbasvir, sofosbuvir, dasabuvir, and combinations thereof.

In some embodiments, the additional therapeutic agent is an influenza virus inhibitor. In some embodiments, the additional therapeutic agent is a matrix 2 inhibitor. For example, in some embodiments, the additional therapeutic agent is selected from the group consisting of amantadine, rimantadine, and combinations thereof. In some embodiments, the additional therapeutic agent is a neuraminidase inhibitor. For example, in some embodiments, the additional therapeutic agent is selected from the group consisting of zanamivir, oseltamivir, peramivir, laninamivir octanoate, and combinations thereof. In some embodiments, the additional therapeutic agent is a polymerase inhibitor. For example, in some embodiments, the additional therapeutic agent is selected from the group consisting of ribavirin, favipiravir, and combinations thereof. In some embodiments, the additional therapeutic agent is selected from the group consisting of amantadine, rimantadine, arbidol (umifenovir), baloxavir marboxil, oseltamivir, peramivir, ingavirin, laninamivir octanoate, zanamivir, favipiravir, ribavirin, DAS-181, XC-221 and combinations thereof. In some embodiments, the additional therapeutic agent is selected from the group consisting of amantadine, rimantadine, zanamivir, oseltamivir, peramivir, laninamivir octanoate, ribavirin, favipiravir, and combinations thereof.

In some embodiments, the additional therapeutic agent is an interferon. In some embodiments, the additional therapeutic agent is selected from the group consisting of interferon alfacon 1, interferon alfa 1b, interferon alfa 2a, interferon alfa 2b, pegylated interferon alfacon 1, pegylated interferon alfa 1b, pegylated interferon alfa 2a (PegIFNα-2a), and PegIFNα-2b. e embodiments, the additional therapeutic agent is selected from the group consisting of interferon alfacon 1, interferon alfa 1b, interferon alfa 2a, interferon alfa 2b, interferon alfa 2 ligand, pegylated interferon alfa 2a (PegIFNα-2a), and PegIFNα-2b. In some embodiments, the additional therapeutic agent is selected from the group consisting of interferon alfacon 1, pegylated interferon alfa 2a (PegIFNα-2a), PegIFNα-2b, and ribavirin. In some embodiments, the additional therapeutic agent is pegylated interferon alfa-2a, pegylated interferon alfa-2b, or a combination thereof. In some examples, the additional therapeutic agent is interferon-beta. For example, the additional therapeutic agent is interferon-beta-1a, such as SNG-001. In some embodiments, the additional therapeutic agent is an interferon-inducing agent, such as tilorone hydrochloride. In some embodiments, the additional therapeutic agent is IL-17 antagonist such as ixekizumab, secukinumab, IMU-838, and vidofludimus.

In some embodiments, the additional therapeutic agent is an immunostimulatory agent. In some embodiments, the additional therapeutic agent is an oligonucleotide. In some embodiments, the additional therapeutic agent is an antimitotic inhibitor. For example, in some embodiments, the additional therapeutic agent is selected from the group consisting of fomivirsen, podofilox imiquimod, sinecatechins, azoximer bromide, IMM-101 and combinations thereof.

In some embodiments, the additional therapeutic agent is selected from the group consisting of besifovir, nitazoxanide, REGN2222, doravirine, sofosbuvir, velpatasvir, daclatasvir, asunaprevir, beclabuvir, FV100, and letermovir, and combinations thereof.

In some embodiments, the additional therapeutic agent is an agent for treatment of RSV. For example, in some embodiments, the antiviral agent is ribavirin, ALS-8112 or presatovir. For example, in some embodiments, the antiviral agent is ALS-8112 or presatovir.

In some embodiments, the antiviral agent is DFV890. In some embodiments, the antiviral agent is MAS825. In some embodiments, the antiviral agent is emetine. In some embodiments, the antiviral agent is virafin. In some embodiments, the antiviral agent is berdazimer sodium. In some embodiments, the antiviral agent is KT-07. In some embodiments, the antiviral agent is iorta-carrageenan. In some embodiments, the antiviral agent is polyoxidonium. In some embodiments, the antiviral agent is bitespiramycin. In some embodiments, the antiviral agent is an anti-Adrenomedullin antibody, such as enibarcimab. In some embodiments, the antiviral agent is an annexin A5 stimulator, such as SY-005.spyke. In some embodiments, the antiviral agent is a COVID19 replicase polyprotein lab inhibitor, such as DC-402234. In some embodiments, the antiviral agent is a host cell factor modulator, such as GBV-006.

In some embodiments, the antiviral agent is protoporphyrin IX, stannous, SnPP protoporphyrin and verteporfin. In some embodiments, the antiviral agent is RBT-9. In some embodiments, the antiviral agent is thymosin.

In some embodiments, the additional therapeutic agent is ivermectin.

In some embodiments, the additional therapeutic agent is an agent for treatment of picornavirus. In some embodiments, the additional therapeutic agent is selected from the group consisting of hydantoin, guanidine hydrochloride, L-buthionine sulfoximine, Py-11, and combinations thereof. In some embodiments, the additional therapeutic agent is a picornavirus polymerase inhibitor. In some embodiments, the additional therapeutic agent is rupintrivir.

In some embodiments, the additional therapeutic agent is an agent for treatment of malaria. For example, the additional therapeutic agent is dihydroartemisinin piperaquine, Pyramax.

In some embodiments, the additional therapeutic agent is selected from the group consisting of hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, cipargamin, artemisone, and combinations thereof.

In some embodiments, the additional therapeutic agent is an agent for treatment of coronavirus. In some embodiments, the additional therapeutic agent is selected from a group consisting of IFX-1, FM-201, CYNK-001, DPP4-Fc, ranpimase, LB-2, AM-1, anti-viroporins, and combinations thereof.

In some embodiments, the additional therapeutic agent is an agent for treatment of ebola virus. For example, in some embodiments, the additional therapeutic agent is selected from the group consisting of ribavirin, palivizumab, motavizumab, RSV-IGIV (RespiGam®), MEDI-557, A-60444, MDT-637, BMS-433771, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), favipiravir (also known as T-705 or Avigan), T-705 monophosphate, T-705 diphosphate, T-705 triphosphate, FGI-106 (1-N,7-N-bis [3-(dimethylamino)propyl]-3,9-dimethylquinolino[8,7-h] quinolone-1,7-diamine), JK-05, TKM-Ebola, ZMapp, rNAPc2, VRC-EBOADC076-00-VP, OS-2966, MVA-BN filo, brincidofovir, Vaxart adenovirus vector 5-based ebola vaccine, Ad26-ZEBOV, FiloVax vaccine, GOVX-E301, GOVX-E302, ebola virus entry inhibitors (NPC1 inhibitors), rVSV-EBOV, and combinations thereof. In some embodiments, the additional therapeutic agent is ZMapp, mAB114, REGEN-EB3, and combinations thereof.

In some embodiments, the additional therapeutic agent is an agent for treatment of HCV. In some embodiments, the additional therapeutic agent is a HCV polymerase inhibitor. For example, in some embodiments, the additional therapeutic agent is selected from the group consisting of sofosbuvir, AT-527, GS-6620, PSI-938, ribavirin, tegobuvir, radalbuvir, MK-0608, and combinations thereof. In some embodiments, the additional therapeutic agent is a HCV protease inhibitor. For example, in some embodiments, the additional therapeutic agent is selected from the group consisting of such as GS-9256, vedroprevir, voxilaprevir, and combinations thereof.

In some embodiments, the additional therapeutic agent is a NS5A inhibitor. For example, in some embodiments, the additional therapeutic agent is selected from the group consisting of ledipasvir, velpatasvir, and combinations thereof.

In some embodiments, the additional therapeutic agent is an anti HBV agent. For example, in some embodiments, the additional therapeutic agent is tenofovir disoproxil fumarate and emtricitabine, or a combination thereof. Examples of additional anti HBV agents include but are not limited to AIC-649, alpha-hydroxytropolones, amdoxovir, antroquinonol, beta-hydroxycytosine nucleosides, ARB-199, CCC-0975, ccc-R08, elvucitabine, ezetimibe, cyclosporin A, gentiopicrin (gentiopicroside), HH-003, hepalatide, JNJ-56136379, CRV-431, nitazoxanide, birinapant, NJK14047, NOV-205 (molixan, BAM-205), oligotide, mivotilate, feron, GST-HG-131, levamisole, Ka ShuNing, alloferon, WS-007, Y-101 (Ti Fen Tai), rSIFN-co, PEG-IIFNm, KW-3, BP-Inter-014, oleanolic acid, HepB-nRNA, cTP-5 (rTP-5), HSK-II-2, HEISCO-106-1, HEISCO-106, Hepbarna, IBPB-0061A, Hepuyinfen, DasKloster 0014-01, ISA-204, Jiangantai (Ganxikang), MIV-210, OB-AI-004, PF-06, picroside, DasKloster-0039, hepulantai, IMB-2613, TCM-800B, reduced glutathione, RO-6864018, RG-7834, QL-007sofosbuvir, ledipasvir, UB-551, and ZH-2N, and the compounds disclosed in US20150210682, (Roche), US 2016/0122344 (Roche), WO2015173164, WO2016023877, US2015252057A (Roche), WO16128335A1 (Roche), WO16120186A1 (Roche), US2016237090A (Roche), WO16107833A1 (Roche), WO16107832A1 (Roche), US2016176899A (Roche), WO16102438A1 (Roche), WO16012470A1 (Roche), US2016220586A (Roche), and US2015031687A (Roche). In some embodiments, the additional therapeutic agent is a HBV polymerase inhibitor. Examples of HBV DNA polymerase inhibitors include, but are not limited to, adefovir (HEPSERA®), emtricitabine (EMTRIVA®), tenofovir disoproxil fumarate (VIREAD®), tenofovir alafenamide, tenofovir, tenofovir disoproxil, tenofovir alafenamide fumarate, tenofovir alafenamide hemifumarate, tenofovir dipivoxil, tenofovir dipivoxil fumarate, tenofovir octadecyloxyethyl ester, CMX-157, tenofovir exalidex, besifovir, entecavir (BARACLUDE®), entecavir maleate, telbivudine (TYZEKA®), filocilovir, pradefovir, clevudine, ribavirin, lamivudine (EPIVIR-HBV®), phosphazide, famciclovir, fusolin, metacavir, SNC-019754, FMCA, AGX-1009, AR-II-04-26, HIP-1302, thymalfasin, tenofovir disoproxil aspartate, tenofovir disoproxil orotate, and HS-10234. In some embodiments, the additional therapeutic agent is a HBV capsid inhibitor.

In some embodiments, the additional therapeutic agent is an agent for treatment of HIV. In some embodiments, the additional therapeutic agent is selected from the group consisting of HIV protease inhibitors, HIV integrase inhibitors, entry inhibitors, HIV nucleoside reverse transcriptase inhibitors, HIV nonnucleoside reverse transcriptase inhibitors, acyclic nucleoside phosphonate analogues, and combinations thereof.

In some embodiments, the additional therapeutic agent is selected from the group consisting of HIV protease inhibitors, HIV non-nucleoside or non-nucleotide inhibitors of reverse transcriptase, HIV nucleoside or nucleotide inhibitors of reverse transcriptase, HIV integrase inhibitors, HIV non-catalytic site (or allosteric) integrase inhibitors, HIV entry inhibitors, HIV maturation inhibitors, immunomodulators, immunotherapeutic agents, antibody-drug conjugates, gene modifiers, gene editors (such as CRISPR/Cas9, zinc finger nucleases, homing nucleases, synthetic nucleases, TALENs), and cell therapies (such as chimeric antigen receptor T-cell, CAR-T, and engineered T cell receptors, TCR-T, autologous T cell therapies, autologous stem cell therapies). In some embodiments, the additional therapeutic agent is an immunotherapeutic peptides such as tertomotide. In some embodiments, the additional therapeutic agent is a CCL26 gene inhibitor, such as mosedipimod. In some embodiments, the additional therapeutic agent is FT-516.

In some embodiments, the additional therapeutic agent is selected from the group consisting of combination drugs for HIV, other drugs for treating HIV, HIV protease inhibitors, HIV reverse transcriptase inhibitors, HIV integrase inhibitors, HIV non-catalytic site (or allosteric) integrase inhibitors, HIV entry (fusion) inhibitors, HIV maturation inhibitors, latency reversing agents, capsid inhibitors, immune-based therapies, PI3K inhibitors (i.e idelalisib, duvelisib), HIV antibodies, and bispecific antibodies, and “antibody-like” therapeutic proteins, and combinations thereof.

In some examples, the additional therapeutic agent is a HIV combination drug. Examples of the HIV combination drugs include, but are not limited to ATRIPLA® (efavirenz, tenofovir disoproxil fumarate, and emtricitabine); BIKTARVY® (bictegravir, emtricitabine, and tenofovir alafenamide); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); SYMTUZA® (darunavir, tenofovir alafenamide hemifumarate, emtricitabine, and cobicistat); SYMFI'm (efavirenz, lamivudine, and tenofovir disoproxil fumarate); CIMDU™ (lamivudine and tenofovir disoproxil fumarate); tenofovir and lamivudine; tenofovir alafenamide and emtricitabine; tenofovir alafenamide hemifumarate and emtricitabine; tenofovir alafenamide hemifumarate, emtricitabine, and rilpivirine; tenofovir alafenamide hemifumarate, emtricitabine, cobicistat, and elvitegravir; COMBIVIR® (zidovudine and lamivudine; AZT+3TC); EPZICOM® (LIVEXA®; abacavir sulfate and lamivudine; ABC+3TC); KALETRA® (ALUVIA®; lopinavir and ritonavir); TRIUMEQ® (dolutegravir, abacavir, and lamivudine); TRIZIVIR® (abacavir sulfate, zidovudine, and lamivudine; ABC+AZT+3TC); atazanavir and cobicistat; atazanavir sulfate and cobicistat; atazanavir sulfate and ritonavir; darunavir and cobicistat; dolutegravir and rilpivirine; dolutegravir and rilpivirine hydrochloride; dolutegravir, abacavir sulfate, and lamivudine; lamivudine, nevirapine, and zidovudine; raltegravir and lamivudine; doravirine, lamivudine, and tenofovir disoproxil fumarate; doravirine, lamivudine, and tenofovir disoproxil; dapivirine+levonorgestrel, dolutegravir+lamivudine, dolutegravir+emtricitabine+tenofovir alafenamide, elsulfavirine+emtricitabine+tenofovir disoproxil, lamivudine+abacavir+zidovudine, lamivudine+abacavir, lamivudine+tenofovir disoproxil fumarate, lamivudine+zidovudine+nevirapine, lopinavir+ritonavir, lopinavir+ritonavir+abacavir+lamivudine, lopinavir+ritonavir+zidovudine+lamivudine, tenofovir+lamivudine, and tenofovir disoproxil fumarate+emtricitabine+rilpivirine hydrochloride, lopinavir, ritonavir, zidovudine and lamivudine.

In some embodiments, the additional therapeutic agent is a HIV protease inhibitor. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat, ASC-09, AEBL-2, MK-8718, GS-9500, GS-1156, and combinations thereof. For example, in some embodiments the additional therapeutic agent is selected from the group consisting of saquinavir, ritonavir, indinavir, nelfinavir, amprenavir, lopinavir, atazanavir, fosamprenavir, darunavir, tipranavir, cobicistat. In some examples, the additional therapeutic agent is selected from the group consisting of amprenavir, atazanavir, brecanavir, darunavir, fosamprenavir, fosamprenavir calcium, indinavir, indinavir sulfate, lopinavir, nelfinavir, nelfinavir mesylate, ritonavir, saquinavir, saquinavir mesylate, tipranavir, GC-376, DG-17, TMB-657 (PPL-100), T-169, BL-008, MK-8122, TMB-607, TMC-310911, and combinations thereof.

In some embodiments, the additional therapeutic agent is a HIV integrase inhibitor. For example, in some embodiment, the additional therapeutic agent is selected from the group consisting of raltegravir, elvitegravir, dolutegravir, abacavir, lamivudine, bictegravir and combinations thereof. In some embodiment, the additional therapeutic agent is bictegravir. In some examples, the additional therapeutic agent is selected from a group consisting of bictegravir, elvitegravir, curcumin, derivatives of curcumin, chicoric acid, derivatives of chicoric acid, 3,5-dicaffeoylquinic acid, derivatives of 3,5-dicaffeoylquinic acid, aurintricarboxylic acid, derivatives of aurintricarboxylic acid, caffeic acid phenethyl ester, derivatives of caffeic acid phenethyl ester, tyrphostin, derivatives of tyrphostin, quercetin, derivatives of quercetin, isoquercetin, raltegravir, dolutegravir, JTK-351, bictegravir, AVX-15567, BMS-986197, cabotegravir (long-acting injectable), diketo quinolin-4-1 derivatives, integrase-LEDGF inhibitor, ledgins, M-522, M-532, NSC-310217, NSC-371056, NSC-48240, NSC-642710, NSC-699171, NSC-699172, NSC-699173, NSC-699174, stilbenedisulfonic acid, T-169, VM-3500, cabotegravir, and combinations thereof.

In some embodiments, the additional therapeutic agent is a HIV entry inhibitor. For example, in some embodiments, the additional therapeutic agent is selected from the group consisting of enfuvirtide, maraviroc, and combinations thereof. Further examples of HIV entry inhibitors include, but are not limited to, cenicriviroc, CCRS inhibitors, gp41 inhibitors, CD4 attachment inhibitors, DS-003 (BMS-599793), gp120 inhibitors, and CXCR4 inhibitors. Examples of CCRS inhibitors include aplaviroc, vicriviroc, maraviroc, cenicriviroc, leronlimab (PRO-140), adaptavir (RAP-101), nifeviroc (TD-0232), EOM-613 anti-GP120/CD4 or CCR5 bispecific antibodies, B-07, MB-66, polypeptide C25P, TD-0680, and vMIP (Haimipu). Examples of CXCR4 inhibitors include plerixafor, ALT-1188, N15 peptide, balixafortide, motixafortide, and vMIP (Haimipu).

In some embodiments, the additional therapeutic agent is a HIV nucleoside reverse transcriptase inhibitors. In some embodiments, the additional therapeutic agent is a HIV nonnucleoside reverse transcriptase inhibitors. In some embodiments, the additional therapeutic agent is an acyclic nucleoside phosphonate analogue. In some embodiments, the additional therapeutic agent is a HIV capsid inhibitor.

In some embodiments, the additional therapeutic agent is a HIV nucleoside or nucleotide inhibitor of reverse transcriptase. For example, the additional therapeutic agent is selected from the group consisting of adefovir, adefovir dipivoxil, azvudine, emtricitabine, tenofovir, tenofovir alafenamide, tenofovir alafenamide fumarate, tenofovir alafenamide hemifumarate, tenofovir disoproxil, tenofovir disoproxil fumarate, tenofovir disoproxil hemifumarate, VIDEX® and VIDEX EC® (didanosine, ddl), abacavir, abacavir sulfate, alovudine, apricitabine, censavudine, didanosine, elvucitabine, festinavir, fosalvudine tidoxil, CMX-157, dapivirine, doravirine, etravirine, OCR-5753, tenofovir disoproxil orotate, fozivudine tidoxil, islatravir, lamivudine, phosphazid, stavudine, zalcitabine, zidovudine, rovafovir etalafenamide (GS-9131), GS-9148, MK-8504, MK-8591, MK-858, VM-2500, KP-1461, and combinations thereof.

In some examples, the additional therapeutic agent is a HIV non-nucleoside or non-nucleotide inhibitor of reverse transcriptase. For example, the additional agent is selected from the group consisting of dapivirine, delavirdine, delavirdine mesylate, doravirine, efavirenz, etravirine, lentinan, MK-8583, nevirapine, rilpivirine, TMC-278LA, ACC-007, AIC-292, KM-023, PC-1005, elsulfavirine rilp (VM-1500), combinations thereof. In some examples, the additional therapeutic agent is a HIV vaccine, such as DermaVir.

In some embodiments, the additional therapeutic agents are selected from ATRIPLA® (efavirenz, tenofovir disoproxil fumarate, and emtricitabine); COMPLERA® (EVIPLERA®; rilpivirine, tenofovir disoproxil fumarate, and emtricitabine); STRIBILD® (elvitegravir, cobicistat, tenofovir disoproxil fumarate, and emtricitabine); TRUVADA® (tenofovir disoproxil fumarate and emtricitabine; TDF+FTC); DESCOVY® (tenofovir alafenamide and emtricitabine); ODEFSEY® (tenofovir alafenamide, emtricitabine, and rilpivirine); GENVOYA® (tenofovir alafenamide, emtricitabine, cobicistat, and elvitegravir); adefovir; adefovir dipivoxil; cobicistat; emtricitabine; tenofovir; tenofovir disoproxil; tenofovir disoproxil fumarate; tenofovir alafenamide; tenofovir alafenamide hemifumarate; TRIUMEQ® (dolutegravir, abacavir, and lamivudine); dolutegravir, abacavir sulfate, and lamivudine; raltegravir; raltegravir and lamivudine; maraviroc; enfuvirtide; ALUVIA® (KALETRA®; lopinavir and ritonavir); COMBIVIR® (zidovudine and lamivudine; AZT+3TC); EPZICOM® (LIVEXA®; abacavir sulfate and lamivudine; ABC+3TC); TRIZIVIR® (abacavir sulfate, zidovudine, and lamivudine; ABC+AZT+3TC); rilpivirine; rilpivirine hydrochloride; atazanavir sulfate and cobicistat; atazanavir and cobicistat; darunavir and cobicistat; atazanavir; atazanavir sulfate; dolutegravir; elvitegravir; ritonavir; atazanavir sulfate and ritonavir; darunavir; lamivudine; prolastin; fosamprenavir; fosamprenavir calcium efavirenz; etravirine; nelfinavir; nelfinavir mesylate; interferon; didanosine; stavudine; indinavir; indinavir sulfate; tenofovir and lamivudine; zidovudine; nevirapine; saquinavir; saquinavir mesylate; aldesleukin; zalcitabine; tipranavir; amprenavir; delavirdine; delavirdine mesylate; Radha-108 (receptol); lamivudine and tenofovir disoproxil fumarate; efavirenz, lamivudine, and tenofovir disoproxil fumarate; phosphazid; lamivudine, nevirapine, and zidovudine; abacavir; and abacavir sulfate.

In some embodiments, the additional therapeutic agent is selected from the group consisting of colistin, valrubicin, icatibant, bepotastine, epirubicin, epoprosetnol, vapreotide, aprepitant, caspofungin, perphenazine, atazanavir, efavirenz, ritonavir, acyclovir, ganciclovir, penciclovir, prulifloxacin, bictegravir, nelfinavir, tegobuvi, nelfinavir, praziquantel, pitavastatin, perampanel, eszopiclone, and zopiclone.

In some embodiments, the additional therapeutic agent is a COVID19 Spike glycoprotein inhibitor, such as tafoxiparin.

In some embodiments, the additional therapeutic agent is a Furin inhibitor, such as BOS-857, BOS-981, alpha defensins.

In some embodiments, the additional therapeutic agent is a CD73 agonist, such as FP-1201.

In some embodiments, the additional therapeutic agent is a CGRP receptor antagonist, such as BHV-3500.

In some embodiments, the additional therapeutic agent is a Cytochrome P450 3A4 inhibitor/Peptidyl-prolyl cis-trans isomerase A inhibitor, such as alisporivir.

In some embodiments, the additional therapeutic agent is a progesterone receptor agonist, such as Progesterone-IBSA.

In some embodiments, the additional therapeutic agent is a GABA A receptor modulator, such as brexanolone.

In some embodiments, the additional therapeutic agent is an inhibitor of Bruton tyrosine kinase (BTK, AGMX1, AT, ATK, BPK, IGHD3, IMD1, PSCTK1, XLA; NCBI Gene ID: 695). For example, in some embodiments, the additional therapeutic agent is selected from the group consisting of (S)-6-amino-9-(1-(but-2-ynoyl)pyrrolidin-3-yl)-7-(4-phenoxyphenyl)-7H-purin-8(9H)-one, acalabrutinib (ACP-196), abivertinib maleate (STI-5656), zanubrutinib (BGB-3111), CB988, HM71224, ibrutinib (Imbruvica), M-2951 (evobrutinib), M7583, tirabrutinib (ONO-4059), PRN-1008, spebrutinib (CC-292), TAK-020, vecabrutinib, ARQ-531, SHR-1459, DTRMWXHS-12, TAS-5315, TL-895, AZD6738, calquence, danvatirsen, and combinations thereof. In some embodiments, the additional therapeutic agent is selected from a group consisting of tirabrutinib, ibrutinib, acalabrutinib, and combinations thereof. In some embodiments, the additional therapeutic agent is selected from a group consisting of tirabrutinib, ibrutinib, and combinations thereof. In some embodiments, the additional therapeutic agent is a receptor tyrosine kinase inhibitor (RTKI). In some embodiments, the additional therapeutic agent is tyrphostin A9 (A9). In some embodiments, the additional therapeutic agent is a TEK receptor tyrosine kinase inhibitor.

In some embodiments, the additional therapeutic agent is a tyrosine kinase inhibitor, such as masitinib.

In some embodiments, the additional therapeutic agent is a sphingosine kinase-2 (sk2) inhibitor, such as opaganib.

In some embodiments, the additional therapeutic agent is a Syk tyrosine kinase inhibitor, such as fostamatinib disodium.

In some embodiments, the additional therapeutic agent is a cholesterol ester transfer protein inhibitor, such as dalcetrapib.

In some embodiments, the additional therapeutic agent is a kinase inhibitor such as pacritinib.

In some embodiments, the additional therapeutic agent is an Axl tyrosine kinase receptor inhibitor, such as bemcentinib.

In some embodiments, the additional therapeutic agent is a FYVE finger phosphoinositide kinase inhibitor.

In some embodiments, the additional therapeutic agent is a checkpoint kinase inhibitor, such as prexasertib.

In some embodiments, the additional therapeutic agent is a MAP kinase inhibitor, such as KTH-222, ATI-450.

In some embodiments, the additional therapeutic agent is a casein kinase II inhibitor, such as silmitasertib.

In some embodiments, the additional therapeutic agent is a Bcr-Abl tyrosine kinase inhibitor, such as radotinib.

In some embodiments, the additional therapeutic agent is a phospholipase A2 inhibitor, such as icosapent ethyl.

In some embodiments, the additional therapeutic agent is a mTOR inhibitor, such as sirolimus.

In some embodiments, the additional therapeutic agent is a pi3k/mTOR inhibitor such as dactolisib.

In some embodiments, the additional therapeutic agent is a Hsp90 inhibitor, such as ganetespib, ADX-1612.

In some embodiments, the additional therapeutic agent is a MEK inhibitor such as ATR-002.

In some embodiments, the additional therapeutic agent is a topoisomerase II inhibitor, such as etoposide.

In some embodiments, the additional therapeutic agent is an exportin 1 inhibitor, such as selinexor, verdinexor.

In some embodiments, the additional therapeutic agent is a dual inhibitor of PARP1/2 and Tankyrase 1/2, such as stenoparib (2X-121).

In some embodiments, the additional therapeutic agent is a cyclin dependent kinase inhibitor, such as CYC-065, CYC-202, fadraciclib, seliciclib.

In some embodiments, the additional therapeutic agent is a cytosine DNA methyltransferase inhibitor, such as decitabine, azacytidine, DUR-928.

In some embodiments, the additional therapeutic agent is a DHFR inhibitor, such as methotrexate.

In some embodiments, the additional therapeutic agent is a Deoxyribonuclease stimulator, such as Descartes-30.

In some embodiments, the additional therapeutic agent is a Ribonuclease stimulator, such as ranpimase.

In some embodiments, the additional therapeutic agent is an eukaryotic initiation factor 4A1 (eIF4A1) inhibitor, such as zotatifin.

In some embodiments, the additional therapeutic agent is a small ubiquitin related modifier inhibitor, such as TAK-981.

In some embodiments, the additional therapeutic agent is a Ubiquitin ligase modulator, such as KPG-818.

In some embodiments, the additional therapeutic agent is an integrin agonist such as 7HP-349.

In some embodiments, the additional therapeutic agent is a BET inhibitor, such as apabetalone.

In some embodiments, the additional therapeutic agent is a BRD4 inhibitor, such as CPI-0610, ABBV-744.

In some embodiments, the additional therapeutic agent is an ER1 inhibitor, such as toremifene.

In some embodiments, the additional therapeutic agent is a KRAS inhibitor. For example, in some embodiments, the additional therapeutic agent is selected from the group consisting of AMG-510, COTI-219, MRTX-1257, ARS-3248, ARS-853, WDB-178, BI-3406, BI-1701963, ARS-1620 (G12C), SML-8-73-1 (G12C), Compound 3144 (G12D), Kobe0065/2602 (Ras GTP), RT11, MRTX-849 (G12C) and K-Ras(G12D)-selective inhibitory peptides, including KRpep-2 (Ac-RRCPLYISYDPVCRR-NH2), KRpep-2d (Ac-RRRRCPLYISYDPVCRRRR-NH2), and combinations thereof.

In some embodiments, the additional therapeutic agent is a proteasome inhibitor. For example, in some embodiments, the additional therapeutic agent is selected from a group consisting of ixazomib, carfilzomib, marizomib, bortezomib, disulfiram+copper gluconate, and combinations thereof in some embodiments, the additional therapeutic agent is carfilzomib. In some embodiments, the additional therapeutic agent is an alkylating agent, such as melphalan.

In some embodiments, the additional therapeutic agent is a vaccine. For example, in some embodiments, the additional therapeutic agent is a DNA vaccine, RNA vaccine, live-attenuated vaccine, inactivated vaccine (i.e. inactivated SARS-CoV-2 vaccine), therapeutic vaccine, prophylactic vaccine, protein based vaccine, viral vector vaccine, cellular vaccine, dendritic cell vaccine (i.e. LV-SMENP-DC, LV-SMENP-DC, or AV-COVID-19) or a combination thereof. In some embodiments, the additional therapeutic agent is mRNA-1273, mRNA-1273.211, mRNA-1273.351, mRNA-1283, CVnCoV, DS-56707, SP-0254, ARCoV, Nanocovax-. In some embodiments, the additional therapeutic agent is INO-4800 or INO-4700. In some embodiments, the therapeutic agent is a DNA vaccine, such as AG301-COVID19, bacTRL-Spike, GX-19, AG-0301-COVID19, ZyCoC-D, GLS-5310, CORVax. In some embodiments, the additional therapeutic agent is live-attenuated RSV vaccine MEDI-559, human monoclonal antibody REGN2222 against RSV, palivizumab, respiratory syncytial virus immune globulin, intravenous [RSV-IGIV], and combinations thereof. In some embodiments, the additional therapeutic agent is an HBV vaccine, for example pediarix, engerix-B, and recombivax HB. In some embodiments, the additional therapeutic agent is a VZV vaccine, for example zostavax and varivax. In some embodiments, the additional therapeutic agent is a HPV vaccine, for example cervarix, gardasil 9, and gardasil. In some embodiments, the additional therapeutic agent is an influenza virus vaccine. For example, a (i) monovalent vaccine for influenza A (e.g. influenza A [H5N1] virus monovalent vaccine and influenza A [H1N1] 2009 virus monovalent vaccines), (ii) trivalent vaccine for influenza A and B viruses (e.g. Afluria, Agriflu, Fluad, Fluarix, Flublok, Flucelvax, FluLaval, Fluvirin, and Fluzone), and (iii) quadrivalent vaccine for influenza A and B viruses (FluMist, Fluarix, Fluzone, and FluLaval). In some embodiments, the additional therapeutic agent is a human adenovirus vaccine (e.g. Adenovirus Type 4 and Type 7 Vaccine, Live, Oral). In some embodiments, the additional therapeutic agent is a rotavirus vaccine (e.g. Rotarix for rotavirus serotype G1, G3, G4, or G9 and RotaTeq for rotavirus serotype G1, G2, G3, or G4). In some embodiments, the additional therapeutic agent is a hepatitis A virus vaccine (e.g. Havrix and Vaqta). In some embodiments, the additional therapeutic agent is poliovirus vaccines (e.g. Kinrix, Quadracel, OPV, and Ipol). In some embodiments, the additional therapeutic agent is a yellow fever virus vaccine (e.g. YF-Vax). In some embodiments, the additional therapeutic agent is a Japanese encephalitis virus vaccines (e.g. Ixiaro and JE-Vax). In some embodiments, the additional therapeutic agent is a measles vaccine (e.g. M-M-R II and ProQuad). In some embodiments, the additional therapeutic agent is a mumps vaccine (e.g. M-M-R II and ProQuad). In some embodiments, the additional therapeutic agent is a rubella vaccine (e.g. M-M-R II and ProQuad). In some embodiments, the additional therapeutic agent is a varicella vaccine (e.g. ProQuad). In some embodiments, the additional therapeutic agent is a rabies vaccine (e.g. Imovax and RabAvert). In some embodiments, the additional therapeutic agent is a variola virus (smallpox) vaccine (ACAM2000). In some embodiments, the additional therapeutic agent is a and hepatitis E virus (HEV) vaccine (e.g. HEV239). In some embodiments, the additional therapeutic agent is a MERS vaccine (e.g. MVA-MERS-S, VTP-500). In some embodiments, the additional therapeutic agent is a BCG vaccine. In some embodiments, the additional therapeutic agent is a recombinant protein subunit vaccine (e.g. ZF-2001), EuCorVAc-19, GBP-510, Sinopharma vaccine, SpyCatcher vaccine, SP-0253, VBI-2902, UB-612, MVC-COV1901. In some embodiments, the additional therapeutic agent is a live attenuated bacterial vaccine (e.g. MV-130). In some embodiments, the additional therapeutic agent is a recombinant non-replicating vaccine (e.g. JNJ78436735 (Ad26 SARS-CoV-2)). In some embodiments, the additional therapeutic agent is poly-TLR agonist polyantigenic vaccine (e.g. Mycobacterium w).

In some embodiments, the additional therapeutic agent is a QAZCOVID-IN vaccine. In some embodiments, the additional therapeutic agent is a GRAd-COV2 vaccine. In some embodiments, the additional therapeutic agent is a EpiVacCorona vaccine. In some embodiments, the additional therapeutic agent is a 2019-nCov vaccine. In some embodiments, the additional therapeutic agent is Ad5-nCoV. In some embodiments, the additional therapeutic agents is the mRNA vaccine CV-07050101, BNT-162, BNT162a1, BNT162b1, BNT162b2, BNT-162b3, BNT-162b1, BNT162c2 (prime/boost, single or multiple doses), SW-0123, CoV-2 SAM (LNP) vaccine, PTX-COVID19-B. In some embodiments, the additional agent is a self-replicating RNA vaccine, such as EXG-5003. In some embodiment, the additional agent is AZD1222 (ChAdOxl nCov-19) vaccine. In some embodiments, the additional agent is Gam-COVID-Vac (Ad26), Gam-COVID-Vac (Ad5), Gam-COVID-Vac (Ad26 Prime-boost), Sputnik-Light vector vaccine (rAd26), Covax-19, NasoVAX, NDV-HXP-S vaccine, AdCOVID, VSV-vector based vaccine. In some embodiments, the additional therapeutic agents is TiterQuil-1055 adjuvanted vaccine. In some embodiments, the additional therapeutic agents is LUNAR-COV19 (ARCT-021), TerraCoV2. In some embodiments, the additional agent is COVID-19 S-Trimer. In some embodiments, the additional agent is TNX-1810, and/or TNX-1820, and/or TNX-1830. In some embodiments, the additional agent is VaxiPatch COVID-19 vaccine. In some embodiments, the additional agent is VBI-2901. In some embodiments, the additional agent is VLA-2001. In some embodiments, the additional agent is exoVACC-SARS-CoV2. In some embodiments, the additional agent is SCB-2019. In some embodiments, the additional agent is MV-SARS-CoV-2. In some embodiments, the additional agent is NVX-CoV2373, Matrix-M and NVX-CoV2373. In some embodiments, the additional agent is BBV152A, B, C, PicoVacc, KBP-COVID-19, MF59 adjuvanted SARS-CoV-2 Sclamp, MVC-COV1901, SCB-2019 (COVID-19 S-Trimer+CpG1018+AS03), TMV-083, V-591, VPM1002, V-SARS7, AdCLD-Cov19, AKS-452, BVRS-GamVac, BVRS-GamVac-Combi, CIGB-2020, COVAC-2, FINLAY-FR-1, KD-414, S-268019, T-COVID, CDX-005, COH-0451, ABNCoV2, ERUCOV-VAC, fakhravac, Kocak-19 inaktif adjuvanli COVID-19 vaccine, NBP-2001, CoVepiT, VXA-CoV2-1, CoVac-1, AT-301, LNP-nCoVsaRNA, AdimrSC-2f, BBV-154, COVID-19 XWG-03, FINLAY-FR-2, MV-014-212, MVA-SARS-2-S, RAZI Cov Pars, SPFN_1B-06-PL, V-590, Ad5-Covid-S/N, CORAL.

In some embodiments, the additional therapeutic agent is an antibody, for example a monoclonal antibody. For example, the additional therapeutic agent is an antibody against 2019-nCov selected from the group consisting of the Regeneron antibodies, the Wuxi Antibodies, the Vir Biotechnology Antibodies, antibodies that target the SARS-CoV-2 spike protein, antibodies that can neutralize SARS-CoV-2 (SARS-CoV-2 neutralizing antibodies), and combinations thereof. In some embodiments, the additional therapeutic agent is anti-SARS CoV antibody CR-3022. In some embodiments, the additional therapeutic agent is aPD-1 antibody. In some embodiments, the additional therapeutic agent is anti-IL-6R mAb. For example, the additional therapeutic agent is TZLS-501 or siltuximab. In some embodiments, the additional therapeutic agent is an antibody that targets specific sites on ACE2. In some embodiments, the additional therapeutic agent is a polypeptide targeting SARS-CoV-2 spike protein (S-protein). In some embodiments, the additional therapeutic agent is a virus suppressing factor (VSF, HzVSFv13).

In some embodiments, the additional therapeutic agent is an anti-CD147 antibody. For example, the additional therapeutic agent is meplazumab.

In some embodiments, the additional therapeutic agent is a phosphodiesterase type 4 (PDE4) or phosphodiesterase type 5 (PDE5) inhibitor. In some embodiments, the additional therapeutic agent is a PDE5 inhibitor, for example, the additional therapeutic agent is sildenafil. In some embodiments, the additional therapeutic agent is a PDE3/PDE4 inhibitor, for example, the additional therapeutic agent is brilacidin, ensifentrine.

In some embodiments, the additional therapeutic agent is an agent targeting NKGA2. In some embodiments, the additional therapeutic agent is a checkpoint inhibitor. In some embodiments, the additional therapeutic agent is NKG2 A B activating NK receptor antagonist, such as monalizumab. In some examples, the additional therapeutic agent is a CTLA-4 checkpoint inhibitor, such as BPI-002.

In some embodiments, the additional therapeutic agent is a CD73 antagonist, such as CPI-006, AK-119.

In some embodiments, the additional therapeutic agent is recombinant cytokine gene-derived protein injection. In some embodiments, the additional therapeutic agent is amnion-derived cellular cytokine solution, such as ST-266.

In some embodiments, the additional therapeutic agent is a polymerase inhibitor. In some embodiments, the additional therapeutic agent is a DNA polymerase inhibitor. For example, in some embodiments, the additional therapeutic agent is cidofovir. In some embodiments, the additional therapeutic agent is a RNA polymerase inhibitor. For example, in some embodiments, the additional therapeutic agent is selected from the group consisting of AT-527, ribavirin, favipiravir, lamivudine, galidesivir, pimodivir and combination thereof.

In some embodiments, the additional therapeutic agent is selected from the group consisting of lopinavir, ritonavir, interferon-alpha-2b, ritonavir, arbidol, hydroxychloroquine, darunavir and cobicistat, abidol hydrochloride, oseltamivir, litonavir, emtricitabine, tenofovir alafenamide fumarate, baloxavir marboxil, ruxolitinib, and combinations thereof.

In some embodiments, the additional therapeutic agent is a beta-catenin inhibitor. For example, the additional therapeutic agent is tetrandrine.

In some embodiments, the additional therapeutic agent is a trypsin inhibitor, for example the additional therapeutic agent is ulinastatin, TAK-671.

In some embodiments, the additional therapeutic agent is selected from the group consisting of ABBV-744, dBET6, MZ1, CPI-0610, Sapanisertib, Rapamycin, Zotatifin, Verdinexor, Chloroquine, Dabrafenib, WDB002, Sanglifehrin A, FK-506, Pevonedistat, Ternatin 4, 4E2RCat, Tomivosertib, PS3061, IHVR-19029, Captopril, Lisinopril, Camostat, Nafamostat, Chloramphenicol, Tigecycline, Linezolid, and combinations thereof.

In some embodiments, the additional therapeutic agent is selected form the group consisting of JQ-1, RVX-208, silmitasertib, TMCB, apicidin, valproic acid, Bafilomycin A1, E-52862, PD-144418, RS-PPCC, PD28, haloperidol, entacapone, indomethacin, LTX-109, MAS-825, Metformin, Metformin glycinate, MRG-001, Medregen, MRx-0004, thimerosal, Ponatinib, H-89, Merimepodib, Migalastat, Mycophenolic acid, Ribavirin, XL413, CCT 365623, Midostaurin, Ruxolitinib, ZINC1775962367, ZINC4326719, ZINC4511851, ZINC95559591, AC-55541, AZ8838, Daunorubicin, GB110, S-verapamil, AZ3451, and combinations thereof.

In some embodiments, the additional therapeutic agent is a drug targeting the coronavirus main protease 3CLpro (e.g. lopinavir). In some embodiments the additional therapeutic agent is a drug targeting the papain-like protease PLpro (e.g. lopinavir). In some examples, the additional therapeutic agent is a drug that functions as a virus-host cell fusion inhibitor to prevent viral entry into host cells (e.g. arbidol). In some embodiments, the additional therapeutic agent is a TMPRSS2 inhibitor (e.g. camostat mesylate).

In some embodiments, the additional therapeutic agent is a serine protease inhibitor, such as LB1148, upamostat, RHB-107, alpha-1 antitrypsin, tranexamic acid.

In some embodiments, the additional therapeutic agent is a replicase polyprotein 1a inhibitor/replicase polyprotein lab inhibitor/protease inhibitor/coronavirus 3C protease like inhibitor, such as PF-07304814.

In some embodiments, the additional therapeutic agent is a SARS coronavirus 3C protease like inhibitor, such as PF-07321332.

In some embodiments, the additional therapeutic agent is a serine protease inhibitor, such as DS-2319, repurposed nafamostat mesylate.

In some embodiments, the additional therapeutic agent is a serine protease inhibitor/Transmembrane serine protease 2 inhibitor, such as nafamostat.

In some embodiments, the additional therapeutic agent is a cysteine protease inhibitor, such as SLV-213.

In some embodiments, the additional therapeutic agent is a serine threonine protein kinase ATR inhibitor, such as berzosertib.

In some embodiments, the additional therapeutic agent is an inhibitor of neutrophil elastase, such as lonodelestat.

In some embodiments, the additional therapeutic agent is an α-ketoamide.

In some examples, the additional therapeutic agent is a poly-ADP-ribose polymerase 1 (PARP1) inhibitor, for example, the additional therapeutic agent is CVL218.

In some embodiments, the additional therapeutic agent is selected from the group consisting of 6′-fluorinated aristeromycin analogues, acyclovir fleximer analogues, disulfiram, thiopurine analogues, ASCO9F, CNM-AgZn-17, genistein, JAN-101, nitric oxide (inhalant), nitric oxide based antiviral formulation (oral), RTD-1, PrEP-001, QBKPN, RUTI, GC376, GC813, phenylisoserine derivatives, neuroiminidase inhibitor analogues, pyrithiobac derivatives, bananins and 5-hydroxychromone derivatives, SSYA10-001, griffithsin, HR2P-M1, HR2P-M2, P21S10, Dihydrotanshinone E-64-C and E-64-D, OC43-HR2P, MERS-SHB, 229E-HR1P, 229E-HR2P, resveratrol, 1-thia-4-azaspiro[4.5] decan-3-one derivatives, S-1226, gemcitabine hydrochloride, loperamide, recombinant interferons, cyclosporine A, alisporivir, imatinib mesylate, dasatinib, selumetinib, trametinib, rapamycin, saracatinib, chlorpromazine, triflupromazine, fluphenazine, thiethylperazine, promethazine, cyclophilin inhibitors, K11777, camostat, k22, teicoplanin derivatives, benzo-heterocyclic amine derivatives N30, mycophenolic acid, silvestrol, and combinations thereof.

In some embodiments, the additional therapeutic agent is an anti-CD147 antibody. For example, the additional therapeutic agent is meplazumab.

In some embodiments, the additional therapeutic agent is an antibody that binds to a coronavirus, for example an antibody that binds to SARS or MERS. In some embodiments, the additional therapeutic agent is a 2019-nCoV virus antibody. In some embodiments, the antibody is ABBV-47D11. In some embodiments, the antibody is COVI-GUARD. In some embodiments, the antibody is C144-LS+C135-LS. In some embodiments, the antibody is DXP-604. In some embodiments, the antibody is JMB-2002. In some embodiments, the antibody is LY-CovMab. In some embodiments, the antibody is LY-CoV555. In some embodiments, the antibody is 5309. In some embodiments, the antibody is SAB-185. In some embodiments, the antibody is SI-F019. In some embodiments, the antibody is CB6. In some embodiments, the antibody is COR-101. In some embodiments, the antibody is STI-1499. In some embodiments, the antibody is JS016. In some embodiments, the antibody is VNAR. In some embodiments, the antibody is VIR-7832 and/or VIR-7831. In some embodiments, the antibody is REGN-COV2 (casirivimab+imdevimab or REGN10933+RGN10987). In some embodiments, the antibody is BAT2020, BAT2019. In some embodiments, the antibody is 47D11. In some embodiments, the antibody cocktail is COVI-SHIELD. In some embodiments, the antibody is BRII-196, BRII-198. In some embodiments, the antibody is ADG-20. In some embodiments, the antibody is ABP-300. In some embodiments, the antibody is BI-767551. In some embodiments, the antibody is GSK-4182136. In some embodiments, the antibody is AZD-7442. In some embodiments, the antibody is regdanvimab. In some embodiments, the antibody is etesevimab. In some embodiments, the antibody is SAB-301. In some embodiments, the antibody is A0D-01. In some embodiments, the antibody is COVI-AMG. In some embodiments, the antibody is MW-33. In some embodiments, the antibody is DXP-593. In some embodiments, the antibody is BSVEQAb. In some embodiments, the antibody is anti-SARS-CoV-2 IgY. In some embodiments, the antibody is COVID-EIG. In some embodiments, the antibody is CSL-760. In some embodiments, the antibody is REGN-3048-3051. In some embodiments, the antibody is ADM-03820. In some embodiments, the antibody is HFB-30132A. In some embodiments, the additional therapeutic agent is an anti-Hemolysin alpha antibody, such as tosatoxumab. In some embodiments, the additional therapeutic agent is an anti-LPS antibody IMM-124-E. In some embodiments, the antibody is INM-005, SCTA01, TY-027, XAV-19.

Compositions of the invention are also used in combination with other active ingredients. For the treatment of 2019-nCoV virus infections, preferably, the other active therapeutic agent is active against coronavirus infections, for example 2019-nCoV virus infections. The crystalline forms and compositions of the present invention are also intended for use with general care provided patients with 2019-nCoV viral infections, including parenteral fluids (including dextrose saline and Ringer's lactate) and nutrition, antibiotic (including metronidazole, amphotericin B, amoxicillin/clavulanate, trimethoprim/sulfamethoxazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K, vitamin D, cholecalciferol, vitamin C and zinc sulfate), anti-inflammatory agents (such as ibuprofen or steroids), corticosteroids such as methylprednisolone, prednisone, mometasone, immonumodulatory medications (e.g. interferon), other small molecule or biologics antiviral agents targeting 2019-nCoV (such as but not limited to lopinavir/ritonavir, EIDD-1931, favipiravir, ribavirine, neutralizing antibodies, etc.), vaccines, pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis. In some embodiments, the additional therapeutic agent is dihydroartemisinin/piperaquine. In some examples, the additional therapeutic agent is a corticosteroid, for example the additional therapeutic agent is ciclesonide, budesonide. In some embodiments, the additional therapeutic agent is EIDD-2801 (MH-4482, Molnupiravir).

In some embodiments, the crystalline forms disclosed herein are used in combination with inhibitors such as Panaphix (PAX-1), which inhibit production of pro-inflammatory cytokines. In some embodiments, the crystalline forms disclosed herein are used in combination with inhibitors such as NCP-112 which inhibit excessive immune response such as cytokine storm.

In some embodiments, the additional therapeutic agent is an antifungal agent, for example itraconazole or 17-OH-itraconazole.

In some examples, the additional therapeutic agent is an immunomodulator. Examples of immune-based therapies include toll-like receptors modulators such as tlr1, tlr2, tlr3, tlr4, tlr5, tlr6, tlr7, tlr8, tlr9, tlr10, tlr11, tlr12, and tlr13; programmed cell death protein 1 (Pd-1) modulators (i.e. nivolumab); programmed death-ligand 1 (Pd-L1) modulators (i.e. camrelizumab, pembrolizumab); IL-15 modulators; interleukin-7 modulators (i.e. efineptakin alfa, plaquenil (hydroxychloroquine), CYT-107); proleukin (aldesleukin, IL-2); interferon alfa; interferon alfa-2b; interferon alfa-n3; lactoferrin, ozanimod, pegylated interferon alfa; interferon gamma; hydroxyurea; mycophenolate mofetil (MPA) and its ester derivative mycophenolate mofetil (MMF); ribavirin; polymer polyethyleneimine (PEI); gepon; IL-12; WF-10; VGV-1; MOR-22; BMS-936559; interleukin-15/Fc fusion protein, AM-0015, ALT-803, NIZ-985, NKTR-255, NKTR-262, NKTR-214, normferon, peginterferon alfa-2a, peginterferon alfa-2b, peginterferon lambda-1a, recombinant interleukin-15, Xmab-24306, RPI-MN, STING modulators, RIG-I modulators, NOD2 modulators, SB-9200, and IR-103. In some embodiments, the additional therapeutic agent is fingolimod, leflunomide, or a combination thereof. In some embodiments, the additional therapeutic agent is thalidomide. In some embodiments, the additional therapeutic agent is CD24Fc. In some embodiments, the additional therapeutic agent is a type I IL-1 receptor antagonists, such as anakinra, astegolimab (MSTT1041A, RG-6149), UTTR1147A.

In some embodiments, the additional therapeutic agent is Ampligen.

In some embodiments, the additional therapeutic agent is lefitolimod.

In some embodiments, the additional therapeutic agent is gamunex.

In some embodiments, the additional therapeutic agent is a CD3 antagonist, such as foralumab.

In some embodiments, the additional therapeutic agent is a KEAP1 modulator, such as SFX-01.

In some embodiments, the additional therapeutic agent is a PARP inhibitor, such as BGP-15.

In some embodiments, the additional therapeutic agent is octagam.

In some embodiments, the additional therapeutic agent is RPH-104. In some embodiments, the additional therapeutic agent is canakinumab.

In some embodiments, the additional therapeutic agent is a leukocyte Ig like receptor A4 modulator, such as daxdilimab.

In some embodiments, the additional therapeutic agent is a Melanocortin MC1 receptor agonist, such as PL-8177.

In some embodiments, the additional therapeutic agent is an IL-33 ligand inhibitor such as MEDI3506.

In some embodiments, the additional therapeutic agent is an IL-5 receptor antagonist, such as mepolizumab.

In some embodiments, the additional therapeutic agent is an IL-12/IL23 inhibitor, such as apilimod, apilimod dimesylate.

In some embodiments, the additional therapeutic agent is a IL-15 receptor agonist, such as N-803.

In some embodiments, the additional therapeutic agent is an IL-18 ligand inhibitor, such as tadekinig-alfa.

In some embodiments, the additional therapeutic agent is an IL-22 agonist, such as efmarodocokin alfa, F-652.

In some embodiments, the additional therapeutic agent is an interferon gamma ligand inhibitor, such as emapalumab.

In some embodiments, the additional therapeutic agent is an IL-6 inhibitor, for example tocilizumab, sarilumab, olokizumab, sirukumab, clazakizumab, levilimab or a combination thereof. In some embodiments, the additional therapeutic agent is tocilizumab biosimilar (e.g. CMAB-806).

In some embodiments, the additional therapeutic agent is Apolipoprotein B modulator/IL-6 receptor antagonist/Serum amyloid A protein modulator/Transthyretin modulator. For example, the additional agent is Amilo-5MER.

In some embodiments, the additional therapeutic agent is a Melanocortin MC1/MC3 receptor agonist. For example, the additional therapeutic agent is AP-1189.

In some embodiments, the additional therapeutic agent is a NLRP3 inflammasome inhibitor. In some embodiments, the additional therapeutic agent is dapansutrile, DFV-890.

In some embodiments, the additional therapeutic agent is a nicotinamide phosphoribosyltransferase inhibitors. For example, the additional therapeutic agent is enamptcumab.

In some embodiments, the additional therapeutic agent is a dipeptidase 1 (DPEP-1) inhibitor. For example, the additional therapeutic agent is Metablok (LSALT peptide).

In some embodiments, the additional therapeutic agent is an anti-TNF inhibitor. For example, the additional therapeutic agent is adalimumab, etanercept, golimumab, infliximab, or a combination thereof.

In some embodiments, the additional therapeutic agent is a TNF alpha ligand inhibitor, such as XPro1595.

In some embodiments, the additional therapeutic agent is a JAK inhibitor, for example the additional therapeutic agent is baricitinib, filgotinib, tofacitinib, olumiant, TD-0903 or a combination thereof. In some examples, the additional therapeutic agent is jaktinib.

In some embodiments, the additional therapeutic agent is an inflammation inhibitor, for example pirfenidon, LYT-100.

In some embodiments, the additional therapeutic agent is anti-inflammatory agent, such as dociparstat sodium, eicosapentaenoic acid, didodecyl methotrexate, rabeximod, EG-009.

In some embodiments, the additional agent is a TREM receptor 1 antagonist.

In some embodiments, the additional therapeutic agent is a CCR1 antagonist, such as MLN-3897.

In some embodiments, the additional therapeutic agent is a Complement C3 inhibitor, such as NGM-621, AMY-101.

In some embodiments, the additional therapeutic agent is a Complement C1s subcomponent inhibitor, such as RLS-0071.

In some embodiments, the additional therapeutic agent is a Complement factor C2 modulator, such as ARGX-117.

In some embodiments, the additional therapeutic agent is a Galectin-3 inhibitor, such as belapectin.

In some embodiments, the additional therapeutic agent is a heparanase inhibitor, such as tridecasodium pixatimod.

In some embodiments, the additional therapeutic agent is an anti-MASP2 antibody, such as narsoplimab.

In some embodiments, the additional therapeutic agent is a calcium channel modulator, such as dantrolene sodium.

In some embodiments, the additional therapeutic agent is a sodium channel stimulator, such as solnatide.

In some embodiments, the additional therapeutic agent is a alkaline phosphatase stimulator such as bovine alkaline phosphatase.

In some embodiments, the additional therapeutic agent is a complement factor D inhibitor, such as ACH-0144471.

In some embodiments, the additional therapeutic agent is a NK1 antagonist, such as LY-686017.

In some embodiments, the additional therapeutic agent is a Zonulin inhibitor, such as larazotide acetate.

In some embodiments, the additional therapeutic agent is a stem cell antigen-1 inhibitor, such as ampion.

In some embodiments, the additional therapeutic agent is a dual complement C5 factor/Leukotriene BLT receptor antagonist, such as nomacopan.

In some embodiments, the additional therapeutic agent is a superoxide dismutase stimulator, such as avasopasem manganese.

In some embodiments, the additional therapeutic agent is an opioid receptor antagonist, such as naltrexone.

In some embodiments, the additional therapeutic agent is an opioid receptor agonist, such as metenkefalin.

In some embodiments, the additional therapeutic agent is a BMP10/BMP15 gene inhibitor, such as lucinactant.

In some embodiments, the additional therapeutic agent is an actin antagonist, such as gelsolin.

In some embodiments, the additional therapeutic agent is a CD95 antagonist, such as asunercept.

In some embodiments, the additional therapeutic agent is a Fractalkine ligand (CX3CL1) inhibitor, such as quetmolimab.

In some embodiments, the additional therapeutic agent is a Platelet glycoprotein VI (GPVI) inhibitor, such as glenzocimab.

In some embodiments, the additional therapeutic agent targets IKKβ and NFκβ, such as OP-101.

In some embodiment, the additional therapeutic agent is a glucocorticoid receptor agonist, such as hydrocortisone, dexamethasone, dexamethasone phosphate.

In some embodiment, the additional therapeutic agent is a PDGF receptor antagonist/TGF beta receptor antagonist/p38 MAP kinase inhibitor, such as deupirfenidone.

In some embodiment, the additional therapeutic agent is a PGD2 antagonist, such as asapiprant.

In some embodiment, the additional therapeutic agent is a prostaglandin E synthase-1 inhibitor, such as sonlicromanol hydrochloride.

In some embodiment, the additional therapeutic agent is a superoxide dismutase modulator, such as Tempol.

In some embodiment, the additional therapeutic agent is a TLR-4 agonist, such as REVTx-99.

In some embodiment, the additional therapeutic agent is a TLR-2/TLR-4 antagonist, such as VB-201.

In some embodiment, the additional therapeutic agent is a TLR-7/TLR-8 antagonist, such as M-5049.

In some embodiments, the additional therapeutic agent is an immunosuppressant, such as tacrolimus, BXT-10, ibudilast, FP-025, apremilast, abatacept, crizanlizumab, itolizumab, bardoxol one methyl.

In some embodiments, the additional therapeutic agent is a RIP-1 kinase inhibitor, such as DNL-758.

In some embodiments, the additional therapeutic agent is an IL-8 receptor antagonist, such as BMS-986253 (HuMax-IL8), DF-1681 (reparixin).

In some embodiments, the additional therapeutic agent is a CD14 inhibitor, such as IC-14, atibuclimab.

In some embodiments, the additional therapeutic agent is a cyclophilin A inhibitor, such as CRV-431.

In some embodiments, the additional therapeutic agent is a Dihydroorotate dehydrogenase (DHODH) inhibitor, such as brequinar, PCT-299, ASLAN-003.

In some embodiments, the additional therapeutic agent is a G-protein coupled bile acid receptor 1 agonist (GPCR19) agonist, such as HY-209.

In some embodiments, the additional therapeutic agent is a Grp78 calcium binding protein inhibitor/Jun N terminal kinase inhibitor/Transferrin modulator/p38 MAP kinase modulator, such as IT-139.

In some embodiments, the additional therapeutic agent is a Histone deacetylase-6 (HDAC-6) inhibitor, such as CKD-506.

In some embodiments, the additional therapeutic agent is a Lyn tyrosine kinase stimulator, such as tolimidone.

In some embodiments, the additional therapeutic agent is a Tek tyrosine kinase receptor stimulator, such as AV-001.

In some embodiments, the additional therapeutic agent is an Integrin alpha-V/beta-1 and alpha-V/beta-6 antagonist, such as PLN-74809.

In some embodiments, the additional therapeutic agent is an IRAK-4 protein kinase inhibitor, such as PF-06650833.

In some embodiments, the additional therapeutic agent is a plasma kallikrein inhibitor/KLKB1 gene inhibitor, such as IONIS-PKK-LRx.

In some embodiments, the additional therapeutic agent is a Leukocyte elastase inhibitor, such as alvelestat, lonodelestat acetate.

In some embodiments, the additional therapeutic is a Maxi K potassium channel inhibitor, such as ENA-001.

In some embodiments, the additional therapeutic is a Nuclear factor kappa B inhibitor/p38 MAP kinase inhibitor, such as GLS-1027.

In some embodiments, the additional therapeutic is a Nuclear factor kappa B inhibitor such as timbetasin, liposomal curcumin.

In some embodiments, the additional therapeutic is anti-fibrotic, such as RT-1840, nintedanib, GB-0139, nintedanib, pamrevlumab.

In some embodiments, the additional therapeutic is a hepatocyte growth factor (HGF) mimetic, such as SNV-003 (ANG-3777).

In some embodiments, the additional therapeutic agent is an A3 adenosine receptor (A3AR) antagonist, for example the additional therapeutic agent is piclidenoson.

In some embodiments, the additional therapeutic agent is an antibiotic for secondary bacterial pneumonia. For example, the additional therapeutic agent is macrolide antibiotics (e.g. azithromycin, clarithromycin, and Mycoplasma pneumoniae), fluoroquinolones (e.g. ciprofloxacin, besifloxacin and levofloxacin), tetracyclines (e.g. doxycycline and tetracycline), or a combination thereof. In some embodiments, the antibiotic is XEL 1004. In some embodiments, the antibiotic is eravacycline.

In some embodiments, the additional therapeutic agent is a bactericidal permeability protein inhibitor/Outer membrane protein inhibitor, such as RECCE-327.

In some embodiments, the crystalline forms disclosed herein are used in combination with pneumonia standard of care (see e.g. Pediatric Community Pneumonia Guidelines, CID 2011:53 (1 October)). Treatment for pneumonia generally involves curing the infection and preventing complications. Specific treatment will depend on several factors, including the type and severity of pneumonia, age and overall health of the individuals. The options include: (i) antibiotics, (ii) cough medicine, and (iii) fever reducers/pain relievers (for e.g. aspirin, ibuprofen (Advil, Motrin IB, others) and acetaminophen (Tylenol, others)). In some embodiments, the additional therapeutic agent is bromhexine anti-cough.

In some embodiments, the crystalline forms disclosed herein are used in combination with immunoglobulin from cured COVID-19 patients. In some embodiments, the crystalline forms disclosed herein are used in combination with plasma transfusion. In some embodiments, the crystalline forms disclosed herein are used in combination with plasma-derived anti-SARS-CoV-2 IgG. In some embodiments, the crystalline forms disclosed herein are used in combination with TAK-888, NP-028 (anti-SARS-CoV-2 polyclonal hyperimmune globulin (H-IG)), or GC-5131A. In some embodiments, the crystalline forms disclosed herein are used in combination with COVID-19 convalescent plasma or immunoglobulin. In some embodiments, the crystalline forms disclosed herein are used in combination with stem cells. For example, in some embodiments, the crystalline forms disclosed herein are used in combination with AdMSCs, ADR-001, Allo-hMSCs, CAP-1002, hCT-MSC, HB-adMSCs, itMSCs, MultiStem, Pluristem, Remestemcel-L (mesenchymal stem cells), NurOwn®, Rexlemestrocel-L, UCMSCs, or ACT-20.

In some examples, the additional therapeutic agent is an TLR agonist. Examples of TLR agonists include, but are not limited to, vesatolimod (GS-9620), GS-986, IR-103, lefitolimod, tilsotolimod, rintatolimod, polyinosinic-polycytidylic Acid (poly I:C), DSP-0509, AL-034, G-100, MT-2766, cobitolimod, AST-008, motolimod, GSK-1795091, GSK-2245035, VTX-1463, GS-9688, LHC-165, BDB-001, RG-7854, telratolimod.RO-7020531. In some embodiments the additional therapeutic agent is PUL-042.

In some examples, the additional therapeutic agent is selected from the group consisting of AVM-0703, bortezomid, flurazepam, ponatinib, sorafenib, paramethasone, clocortolone, flucloxacillin, sertindole, clevidipine, atorvastatin, simvastatin, trimodulin, rosuvastatin, cinolazepam, clofazimine, fosaprepitant, and combinations thereof.

In some examples, the additional therapeutic agent is carrimycin, suramin, triazavirin, dipyridamole, bevacizumab, meplazumab, GD31 (rhizobium), NLRP inflammasome inhibitor, or α-ketoamine.

In some embodiments, the additional therapeutic agent is viral macrophage inflammatory protein (vMIP).

In some embodiments, the additional therapeutic agent is a recombinant human angiotensin-converting enzyme 2 (rhACE2), for example alunacedase alfa (APN-01), HLX-71. In some embodiments, the additional therapeutic agent is an angiotensin II receptor agonist. In some examples, the additional therapeutic agent is a partial agonist of AT2 or a partial antagonist of AT1. In some embodiments, the additional therapeutic agent is L-163491. In some embodiments, the additional therapeutic agent is valsartan, losartan, candesartan, eprosartan, irbesartan, olmesartan. In some embodiments, the additional therapeutic agent is VP-01, TXA-127. In some embodiments, the additional therapeutic agent is telmisartan.

In some embodiments, the additional therapeutic agent is an ACE inhibitor, such as ramipril, captopril, enalapril, lisonopril.

In some embodiments, the additional therapeutic agent is an Angiotensin II AT-1 receptor antagonist/Beta-arrestin stimulator, such as TRV-027.

In some embodiments, the additional therapeutic agent is an ACE2 inhibitor/COVID19 Spike glycoprotein inhibitor, such as MP-0420.

In some embodiments, the additional therapeutic agent is a caspase inhibitor, such as emricasan.

In some embodiments, the additional therapeutic agent is an acetaldehyde dehydrogenase inhibitor, such as ADX-629.

In some embodiments, the additional therapeutic agent is a dihydroorotate dehydrogenase inhibitor, such as RP-7214.

In some embodiments, the additional therapeutic agent is a dihydroorotate dehydrogenase inhibitor; Protein tyrosine kinase inhibitor, such as repurposed leflunomide.

In some embodiments, the additional therapeutic agent is an aldose reductase inhibitor, such as AT-001.

In some embodiments, the additional therapeutic agent is a platelet inhibitor. For example, the additional therapeutic agent is dipyridamole.

In some embodiments, the additional therapeutic agent is an anti-coagulant, such as heparins (heparin and low molecular weight heparin), aspirin, apixaban, dabigatran, edoxaban, argatroban, enoxaparin, fondaparinux.

In some embodiments, the additional therapeutic agent is a tissue factor inhibitor, such as AB-201.

In some embodiments, the additional therapeutic is a Factor XIIa antagonist, such as garadacimab.

In some embodiments, the additional therapeutic is a Factor XIa antagonist, such as EP-7041.

In some embodiments, the additional therapeutic agent is a VE-PTP inhibitor, such as razuprotafib.

In some embodiments, the additional therapeutic agent is a VIP 2 receptor agonist, such as PB-1046.

In some embodiments, the additional therapeutic agent is an anti-thrombotic, such as defibrotide, rivaroxaban, alteplase, tirofiban, clopidogrel, prasugrel, bemiparin, bivalirudin, sulodexide, tenecteplase.

In some embodiments, the additional therapeutic agent is a vasodilator, such as iloprost, ventaprost, vazegepant, angiotensin 1-7, ambrisentan, NORS, pentoxifylline, propranolol, RESP301, sodium nitrite.

In some embodiments, the additional therapeutic agent is a blood clotting modulator, such as lanadelumab.

In some embodiments, the additional therapeutic agent is a diuretic, such as an aldosterone antagonist, such as spironolactone.

In some embodiments, the additional therapeutic agent is antihypoxic, such as trans-sodium crocetinate.

In some embodiments, the additional therapeutic agent is MK-5475.

In some embodiments, the additional therapeutic agent is a hypoxia-inducible factor (HF) prolyl hydroxylase-2 (PHD-2) inhibitor such as desidustat, vadadustat.

In some embodiments, the additional therapeutic agent is a renin inhibitor, such as aliskiren.

In some embodiments, the additional therapeutic agent is a calcium channel inhibitor such as nifedipine.

In some embodiments, the additional therapeutic agent is a chelating agent, such as desferal, deferiprone, deferoxamine.

In some embodiments, the additional therapeutic agent is a Retinoic acid receptor agonist, such as isotretinoin, or fenretinide.

In some embodiments, the additional therapeutic agent is an AMPA receptor modulator, such as traneurocin (Nanomedivir).

In some embodiments, the additional therapeutic agent is a human antimicrobial peptide, such as LL-37i.

In some embodiments, the additional therapeutic agent is a microbiome modulator, such as EDP-1815, KB-109.

In some embodiments, the additional therapeutic agent is an estrogen receptor antagonist, such as tamoxifen.

In some embodiments, the additional therapeutic agent is an estrogen receptor modulator, such as estetrol.

In some embodiments, the additional therapeutic agent is an androgen receptor antagonist such as bicalutamide, enzalutamide, proxalutamide.

In some embodiments, the additional therapeutic agent is a GNRH receptor antagonist, such as degarelix.

In some embodiments, the additional therapeutic agent is a sex hormone modulator, such as dutasteride.

In some embodiments, the additional therapeutic agent is a thyroid hormone receptor, such as sobetirome.

In some embodiments, the additional therapeutic agent is a calpain inhibitor, such as BLD-2660.

In some embodiments, the additional therapeutic agent is a GM-CSF ligand inhibitor such as gimsilumab, lenzilumab, namilumab, TJM2, otilimab, plonmarlimab.

In some embodiments, the additional therapeutic agent is a GM-CSF receptor antagonist, such as mavrilimumab.

In some embodiments, the additional therapeutic agent is a GM-CSF receptor agonist, such as sargramostim.

In some embodiments, the additional therapeutic agent is an alpha 1 adrenoreceptor antagonist such as prazosin.

In some embodiments, the additional therapeutic agent is a neuropilin 2 inhibitor, such as ATYR-1923.

In some embodiments, the additional therapeutic agent is an activated calcium (CRAC) channel inhibitor, such as CM-4620.

In some embodiments, the additional therapeutic agent is a calcium activated chloride channel (CACC) inhibitor, such as crofelemer.

In some embodiments, the additional therapeutic agent is a proto-oncogene Mas agonist, such as BIO-101.

In some embodiments, the additional therapeutic agent is a DPP4 inhibitor, such as saxagliptin, sitagliptin, alogliptin, linagliptin.

In some embodiments, the additional therapeutic agent is a sodium glucose cotransporter type 2 (SGLT-2) inhibitor such as dapagliflozin propanediol.

In some embodiments, the additional therapeutic agent is a fractalkine receptor inhibitor such as KAND-567.

In some embodiments, the additional therapeutic agent is an alpha2-receptor agonist. For example, the additional therapeutic agent is dexmedetomidine.

In some embodiments, the additional therapeutic agent is a mCBM40 (multivalent carbohydrate-binding module Family 40 domain) product, for example the additional therapeutic agent is Neumifil.

In some embodiments, the additional therapeutic agent is a histamine H1 receptor antagonist, such as ebastine, tranilast.

In some embodiments, the additional therapeutic agent is a histamine H2 receptor antagonist, such as famotidine.

In some embodiments, the additional therapeutic agent is anti-histamine such as cloroperastine, and clemastine.

In some embodiments, the additional therapeutic agent is a vasoactive intestinal peptide receptor 1 agonists, such as aviptadil.

In some embodiments, the additional therapeutic agent is a drug that treats acute respiratory distress syndrome (ARDS), such as FX-06.

In some embodiments, the additional therapeutic agent is BIO-11006.

In some embodiments, the additional therapeutic agent is sodium pyruvate.

In some embodiments, the additional therapeutic agent is LEAF-4L6715, LEAF-4L7520.

In some embodiments, the additional therapeutic agent is a respiratory stimulant, such as almitrine.

In some embodiments, the additional therapeutic agent is a bronchodilator, such as brensocatib, formoterol.

In some embodiments, the additional therapeutic agent is a beta 2 adrenoceptor agonist, such as salmeterol.

In some embodiments, the additional therapeutic agent is hyaluronidase inhibitor such as astodrimer.

In some embodiments, the additional therapeutic agent is an anti-LIGHT antibody, such as CERC-002.

In some embodiments, the additional therapeutic agent is a CRAC (calcium release-activated calcium) channel inhibitor, such as CM-4620-IE.

In some embodiments, the additional therapeutic agent is a TLR4 antagonist, such as EB-05, NI-0101, or E-5564.

In some embodiments, the additional therapeutic agent is a deoxyribonuclease I stimulator, such as GNR-039.

In some embodiments, the additional therapeutic agent is an ornithine decarboxylase inhibitor, such as eflornithine.

In some embodiments, the crystalline forms described herein are used in combination with respiratory-specific small interfering RNA therapies. In some embodiments, these therapies are delivered by a nebulizer.

In some embodiments, the additional therapeutic agent is a vimentin modulator. For example, the additional therapeutic agent is pritumumab, hzVSF-v13.

In some embodiments, the additional therapeutic agent is a modulator of Nsp15 (nonstructural protein 15) such as benzopurpurin B, C-467929, C-473872, AB001, NSC-306711 and N-65828.

In some embodiments, the additional therapeutic agent is a xanthine dehydrogenase inhibitor, such as oxypurinol (XRx-101).

In some embodiments, the additional therapeutic agent is a xanthine oxidase inhibitor, such as bucillamine, Xrx-101.

In some embodiments, the additional therapeutic agent is a cathepsin inhibitor, such as VBY-825, ONO-5334.

In some embodiments, the additional therapeutic agent is a Transforming growth factor beta (TGF-β) inhibitor. For example, the additional therapeutic agent is OT-101.

In some embodiments, the additional therapeutic agent is a N-methyl-D-aspartate (NMDA) receptor antagonist. For example, the additional therapeutic agent is ifenprodil, transcrocetin.

In some embodiments, the additional therapeutic agent is a glycolysis inhibitor. For example, the additional therapeutic agent is WP-1122.

In some embodiments, the additional therapeutic is a Leukotriene D4 antagonist, such as montelukast.

In some embodiments, the additional therapeutic is a Leukotriene BLT receptor antagonist, such as ebselen.

In some embodiments, the additional therapeutic is a tubulin inhibitor, such as VERU-111, colchicine.

In some embodiments, the additional therapeutic agent is a glucosylceramide synthase inhibitor such as miglustat.

In some embodiments, the additional therapeutic agent is a Nrf2 activator, such as PB125.

In some embodiments, the additional therapeutic agent is a Rev protein modulator, such as ABX464.

In some embodiments, the additional therapeutic agent is a nuclear import inhibitor, such as iCP-NI (CV-15).

In some embodiments, the additional therapeutic agent is a cannabinoid CB2 receptor agonist, such as PPP003.

In some embodiments, the additional therapeutic agent is a dehydropeptidase-1 modulator, such as LSALT peptide.

In some embodiments, the additional therapeutic agent is a cyclooxygenase inhibitor, such as celecoxib, naproxen, aspirin/dipyridamole.

In some embodiments, the additional therapeutic agent is an antitoxin such as CAL02.

In some embodiments, the additional therapeutic agent is a nitric oxide stimulant, such as GLS-1200.

In some embodiments, the additional therapeutic agent is an apelin receptor agonist, such as CB-5064.

In some embodiments, the additional therapeutic agent is a complement inhibitor, such as ravulizumab.

In some embodiments, the additional therapeutic agent is a Colony-stimulating factor 1 receptor (CSF1R) inhibitor, such as axatilimab.

In some embodiments, the additional therapeutic agent is a complement C5 factor inhibitor, such as eculizumab, zilucoplan, and C5a such as BDB-001, IFX-1, advoralimab.

In some embodiments, the additional therapeutic agent is a complement C1s inhibitor, such as conestat alpha.

In some embodiment, the additional therapeutic agent is a C3 inhibitor, such as APL-9, AMY-101.

In some embodiments, the additional therapeutic agent is an anti-C5aR antibody, such as advoralimab or vilobelimab.

In some embodiments, the additional therapeutic agent is an anti elongation factor 1 alpha 2 inhibitor, such as plitidepsin.

In some embodiments, the additional therapeutic agent is an angiopoietin ligand-2 inhibitor, such as LY-3127804.

In some embodiments, the additional therapeutic agent is a lysine specific histone demethylase 1 inhibitor, such as vafidemstat.

In some embodiments, the additional therapeutic agent is a histone inhibitor, such as STC-3141.

In some embodiments, the additional therapeutic agent is a hyaluronan inhibitor.

In some embodiments, the additional therapeutic agent is dopamine D2 receptor antagonist, such as chlorpromazine.

In some embodiments, the additional therapeutic agent is a proton pump inhibitor, such as omeprazole.

In some embodiments, the additional therapeutic agent is a PGI2 agonist, such as epoprostenol.

In some embodiments, the additional therapeutic agent is a plasminogen activator inhibitor 1 inhibitor, such asTM-5614.

In some embodiments, the additional therapeutic agent is a Ubiquinol cytochrome C reductase 14 kDa inhibitor, such as telacebec.

In some embodiments, the additional therapeutic agent is an anti-viroporin therapeutic. For example, the additional therapeutic agent is BIT-314 or BIT-225. In some embodiments, the additional therapeutic agent is coronavirus E protein inhibitor. For example, the additional therapeutic agent is BIT-009. Further examples of additional therapeutic agents include those described in WO-2004112687, WO-2006135978, WO-2018145148, and WO-2009018609.

In some embodiments, the additional therapeutic or prophylactic agent is molnupiravir, oseltamivir, nirmatrelvir, or ritonavir. In some embodiments, the additional therapeutic or prophylactic agent is ritonavir or cobicistat. In some embodiments, the additional therapeutic or prophylactic agent is molnupiravir. In some embodiments, the additional therapeutic or prophylactic agent is molnupiravir, oseltamivir, nirmatrelvir, or ritonavir. In some embodiments, the additional therapeutic or prophylactic agent comprises nirmatrelvi and ritonavir. In some embodiments, the additional therapeutic or prophylactic agent comprises nirmatrelvi and cobicistat. In some embodiments, the additional therapeutic or prophylactic agent comprises S-217622.

In some embodiments, the additional therapeutic agent is a cell therapy, such as allogeneic natural killer cells, antigen presenting cells (APC), invariant natural killer T (iNKT) cells, induced pluripotent stem cell (iPSC), allogeneic T-cells, autologous adipose-derived mesenchymal stem cells, allogeneic bone marrow-derived mesenchymal stem cells, allogeneic mesenchymoangioblast-derived mesenchymal stem cells, regulatory T cells (Tregs), dendritic cells. In some embodiments, the additional therapeutic agent is SARS-CoV-2 specific cytotoxic T lymphocyte. In some embodiments, the additional therapeutic agent is agenT-797, Allocetra, ALVR-109, BM-Allo-MSC, CAStem, Cellgram-AKI, CK-0802, CL-2020, IL-15-NK cells, NKG2D-CAR-NK cells, ACE2 CAR-NK cells, DWP-710, partially HLA-matched Virus Specific T cells (VSTs), FT-516, RAPA-501, SARS-CoV-2 Specific T Cells, HLCM-051, ExoFlo, HCR-040, it-hMSC, KI-MSC-PL-205, ORBCEL-C, pathogen-specific aAPC, ProTrans, SBI-101, StemVacs, STI-8282, taniraleucel, UMSC-01.

In some embodiments, the additional therapeutic agent is selected from the group consisting of ABBV-744, dBET6, MZ1, CPI-0610, Sapanisertib, Rapamycin, Zotatifin, Verdinexor, Chloroquine, Dabrafenib, WDB002, Sanglifehrin A, FK-506, Pevonedistat, Ternatin 4, 4E2RCat, Tomivosertib, PS3061, IHVR-19029, XC-7, long-acting injectable ivermectin, Captopril, Lisinopril, Camostat, Chloramphenicol, Tigecycline, Linezolid, and combinations thereof.

In some embodiments, the additional therapeutic agent is selected form a group consisting of tilorone, cannabidiol, cyclosporine, loperamide, mefloquine, amodiaquine, proscillaridin, digitoxin, digoxin, hexachlorophene, hydroxyprogesterone caproate, salinomycin, ouabain, cepharanthine, ciclesonide, oxyclozanide, anidulafungin, gilteritinib, berbamine, tetrandrine, abemaciclib, ivacaftor, bazedoxifene, niclosamide, eltrombopag, and combinations thereof.

In some embodiments, the additional therapeutic agent is selected from the group consisting of hydroxychloroquine, chloroquine, artemether, lumefantrine, atovaquone, proguanil, tafenoquine, pyronaridine, artesunate, artenimol, piperaquine, artesunate, amodiaquine, pyronaridine, artesunate, halofantrine, quinine sulfate, mefloquine, solithromycin, pyrimethamine, MMV-390048, ferroquine, artefenomel mesylate, ganaplacide, DSM-265, ISPM-19, cipargamin, artemisone, and combinations thereof.

It is also possible to combine any crystalline form of the invention with one or more additional active therapeutic agents in a unitary dosage form for simultaneous or sequential administration to a patient. The combination therapy may be administered as a simultaneous or sequential regimen. When administered sequentially, the combination may be administered in two or more administrations.

Co-administration of a crystalline form of the disclosure with one or more other active therapeutic agents generally refers to simultaneous or sequential administration of a crystalline form of the disclosure and one or more other active therapeutic agents, such that therapeutically effective amounts of the crystalline form of the disclosure and one or more other active therapeutic agents are both present in the body of the patient.

Co-administration includes administration of unit dosages of the crystalline forms of the disclosure before or after administration of unit dosages of one or more other active therapeutic agents, for example, administration of the crystalline forms of the disclosure within seconds, minutes, or hours of the administration of one or more other active therapeutic agents. For example, a unit dose of a crystalline form of the disclosure can be administered first, followed within seconds or minutes by administration of a unit dose of one or more other active therapeutic agents. Alternatively, a unit dose of one or more other therapeutic agents can be administered first, followed by administration of a unit dose of a crystalline form of the disclosure within seconds or minutes. In some cases, it may be desirable to administer a unit dose of a crystalline form of the disclosure first, followed, after a period of hours (e.g., 1-12 hours), by administration of a unit dose of one or more other active therapeutic agents. In other cases, it may be desirable to administer a unit dose of one or more other active therapeutic agents first, followed, after a period of hours (e.g., 1-12 hours), by administration of a unit dose of a crystalline form of the disclosure.

The combination therapy may provide “synergy” and “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined formulation; (2) delivered by alternation or in parallel as separate formulations; or (3) by some other regimen. When delivered in alternation therapy, a synergistic effect may be attained when the compounds are administered or delivered sequentially, e.g., in separate tablets, pills or capsules, or by different injections in separate syringes. In general, during alternation therapy, an effective dosage of each active ingredient is administered sequentially, i.e., serially, whereas in combination therapy, effective dosages of two or more active ingredients are administered together. A synergistic anti-viral effect denotes an antiviral effect, which is greater than the predicted purely additive effects of the individual compounds of the combination.

### Combination Therapy for the Treatment of Pneumoviridae

The crystalline forms provided herein are also used in combination with other active therapeutic agents. For the treatment of Pneumoviridae virus infections, preferably, the other active therapeutic agent is active against Pneumoviridae virus infections, particularly respiratory syncytial virus infections and/or metapneumovirus infections. Non-limiting examples of these other active therapeutic agents active against RSV are ribavirin, palivizumab, motavizumab, RSV-IGIV (RespiGam®), MEDI-557, A-60444 (also known as RSV604), MDT-637, BMS-433771, ALN-RSVO, ALX-0171 and mixtures thereof. Other non-limiting examples of other active therapeutic agents active against respiratory syncytial virus infections include respiratory syncytial virus protein F inhibitors, such as AK-0529; RV-521, ALX-0171, JNJ-53718678, BTA-585, and presatovir; RNA polymerase inhibitors, such as lumicitabine and ALS-8112; anti-RSV G protein antibodies, such as anti-G-protein mAb; viral replication inhibitors, such as nitazoxanide.

In some embodiments, the other active therapeutic agent may be a vaccine for the treatment or prevention of RSV, including but not limited to MVA-BN RSV, RSV-F, MEDI-8897, JNJ-64400141, DPX-RSV, SynGEM, GSK-3389245A, GSK-300389-1A, RSV-MEDI deltaM2-2 vaccine, VRC-RSVRGP084-00VP, Ad35-RSV-FA2, Ad26-RSV-FA2, and RSV fusion glycoprotein subunit vaccine.

Non-limiting examples of other active therapeutic agents active against metapneumovirus infections include sialidase modulators such as DAS-181; RNA polymerase inhibitors, such as ALS-8112; and antibodies for the treatment of Metapneumovirus infections, such as EV-046113.

In some embodiments, the other active therapeutic agent may be a vaccine for the treatment or prevention of metapneumovirus infections, including but not limited to mRNA-1653 and rHMPV-Pa vaccine.

### Combination Therapy for the Treatment of Picornaviridae

The crystalline forms provided herein are also used in combination with other active therapeutic agents. For the treatment of Picornaviridae virus infections, preferably, the other active therapeutic agent is active against Picornaviridae virus infections, particularly Enterovirus infections. Non-limiting examples of these other active therapeutic agents are capsid binding inhibitors such as pleconaril, BTA-798 (vapendavir) and other compounds disclosed by Wu, et al. (U.S. Pat. No. 7,078,403) and Watson (U.S. Pat. No. 7,166,604); fusion sialidase protein such as DAS-181; a capsid protein VP1 inhibitor such as VVX-003 and AZN-001; a viral protease inhibitor such as CW-33; a phosphatidylinositol 4 kinase beta inhibitor such as GSK-480 and GSK-533; anti-EV71 antibody.

In some embodiments, the other active therapeutic agent may be a vaccine for the treatment or prevention of Picornaviridae virus infections, including but not limited to EV71 vaccines, TAK-021, and EV-D68 adenovector-based vaccine.

### Combination Therapy for Respiratory Infections

Many of the infections of the Pneumoviridae, Picornaviridae, and Coronaviridae viruses are respiratory infections. Therefore, additional active therapeutics used to treat respiratory symptoms and sequelae of infection may be used in combination with the crystalline forms provided herein. The additional agents are preferably administered orally or by direct inhalation. For example, other preferred additional therapeutic agents in combination with the crystalline forms provided herein for the treatment of viral respiratory infections include, but are not limited to, bronchodilators and corticosteroids.

### Glucocorticoids

Glucocorticoids, which were first introduced as an asthma therapy in 1950 (Carryer, Journal of Allergy, 21, 282-287, 1950), remain the most potent and consistently effective therapy for this disease, although their mechanism of action is not yet fully understood (Morris, J. Allergy Clin. Immunol., 75 (1 Pt) 1-13, 1985). Unfortunately, oral glucocorticoid therapies are associated with profound undesirable side effects such as truncal obesity, hypertension, glaucoma, glucose intolerance, acceleration of cataract formation, bone mineral loss, and psychological effects, all of which limit their use as long-term therapeutic agents (Goodman and Gilman, 10th edition, 2001). A solution to systemic side effects is to deliver steroid drugs directly to the site of inflammation. Inhaled corticosteroids (ICS) have been developed to mitigate the severe adverse effects of oral steroids. Non-limiting examples of corticosteroids that may be used in combinations with the crystalline forms provided herein are dexamethasone, dexamethasone sodium phosphate, fluorometholone, fluorometholone acetate, loteprednol, loteprednol etabonate, hydrocortisone, prednisolone, fludrocortisones, triamcinolone, triamcinolone acetonide, betamethasone, beclomethasone diproprionate, methylprednisolone, fluocinolone, fluocinolone acetonide, flunisolide, fluocortin-21-butylate, flumethasone, flumetasone pivalate, budesonide, halobetasol propionate, mometasone furoate, fluticasone, AZD-7594, ciclesonide; or a pharmaceutically acceptable salts thereof.

### Anti-Inflammatory Agents

Other anti-inflammatory agents working through anti-inflammatory cascade mechanisms are also useful as additional therapeutic agents in combination with the crystalline forms provided herein for the treatment of viral respiratory infections. Applying “anti-inflammatory signal transduction modulators” (referred to in this text as AISTM), like phosphodiesterase inhibitors (e.g., PDE-4, PDE-5, or PDE-7 specific), transcription factor inhibitors (e.g., blocking NEKB through IKK inhibition), or kinase inhibitors (e.g., blocking P38 MAP, JNK, PI3K, EGFR or Syk) is a logical approach to switching off inflammation as these small molecules target a limited number of common intracellular pathways—those signal transduction pathways that are critical points for the anti-inflammatory therapeutic intervention (see review by P. J. Barnes, 2006). These non-limiting additional therapeutic agents include: 5-(2,4-Difluoro-phenoxy)-1-isobutyl-1H-indazole-6-carboxylic acid (2-dimethylamino-ethyl)-amide (P38 Map kinase inhibitor ARRY-797); 3-Cyclopropylmethoxy-N-(3,5-dichloro-pyridin-4-yl)-4-difluorormethoxy-benzamide (PDE-4 inhibitor Roflumilast); 4-[2-(3-cyclopentyloxy-4-methoxyphenyl)-2-phenyl-ethyl]-pyridine (PDE-4 inhibitor CDP-840); N-(3,5-dichloro-4-pyridinyl)-4-(difluoromethoxy)-8-[(methylsulfonyl)amino]-1-dibenzofurancarboxamide (PDE-4 inhibitor Oglemilast); N-(3,5-Dichloro-pyridin-4-yl)-2-[1-(4-fluorobenzyl)-5-hydroxy-1H-indol-3-yl]-2-oxo-acetamide (PDE-4 inhibitor AWD 12-281); 8-Methoxy-2-trifluoromethyl-quinoline-acid (3,5-dichloro-1-oxy-pyridin-4-yl)-amide (PDE-4 inhibitor Sch 351591); 4-[5-(4-Fluorophenyl)-2-(4-methanesulfinyl-phenyl)-1H-imidazol-4-yl]-pyridine (P38 inhibitor SB-203850); 4-[4-(4-Fluoro-phenyl)-1-(3-phenyl-propyl)-5-pyridin-4-yl-1H-imidazol-2-yl]-but-3-yn-1-ol (P38 inhibitor RWJ-67657); 4-Cyano-4-(3-cyclopentyloxy-4-methoxy-phenyl)-cyclohexanecarboxylic acid 2-diethylamino-ethyl ester (2-diethyl-ethyl ester prodrug of Cilomilast, PDE-4 inhibitor); (3-Chloro-4-fluorophenyl)-[7-methoxy-6-(3-morpholin-4-yl-propoxy)-quinazolin-4-yl]-amine (Gefitinib, EGFR inhibitor); and 4-(4-Methyl-piperazin-1-ylmethyl)-N-[4-methyl-3-(4-pyridin-3-yl-pyrimidin-2-ylamino)-phenyl]-benzamide (Imatinib, EGFR inhibitor).

### β2-Adrenoreceptor Agonist Bronchodilators

Combinations comprising inhaled β2-adrenoreceptor agonist bronchodilators such as formoterol, albuterol or salmeterol with the crystalline forms provided herein are also suitable, but non-limiting, combinations useful for the treatment of respiratory viral infections.

Combinations of inhaled β2-adrenoreceptor agonist bronchodilators such as formoterol or salmeterol with ICS's are also used to treat both the bronchoconstriction and the inflammation (Symbicort® and Advair®, respectively). The combinations comprising these ICS and 02-adrenoreceptor agonist combinations along with the crystalline forms provided herein are also suitable, but non-limiting, combinations useful for the treatment of respiratory viral infections.

Other examples of Beta 2 adrenoceptor agonists are bedoradrine, vilanterol, indacaterol, olodaterol, tulobuterol, formoterol, abediterol, salbutamol, arformoterol, levalbuterol, fenoterol, and TD-5471.

### Anticholinergics

For the treatment or prophylaxis of pulmonary broncho-constriction, anticholinergics are of potential use and, therefore, useful as an additional therapeutic agent in combination with the crystalline forms provided herein for the treatment of viral respiratory infections. These anticholinergics include, but are not limited to, antagonists of the muscarinic receptor (particularly of the M3 subtype) which have shown therapeutic efficacy in man for the control of cholinergic tone in COPD (Witek, 1999); 1-{4-Hydroxy-1-[3,3,3-tris-(4-fluoro-phenyl)-propionyl]-pyrrolidine-2-carbonyl}-pyrrolidine-2-carboxylic acid (1-methyl-piperidin-4-ylmethyl)-amide; 3-[3-(2-Diethylamino-acetoxy)-2-phenyl-propionyloxy]-8-isopropyl-8-methyl-8-azonia-bicyclo[3.2.1]octane (Ipratropium-N,N-diethylglycinate); 1-Cyclohexyl-3,4-dihydro-1H-isoquinoline-2-carboxylic acid 1-aza-bicyclo[2.2.2]oct-3-yl ester (Solifenacin); 2-Hydroxymethyl-4-methanesulfinyl-2-phenyl-butyric acid 1-aza-bicyclo[2.2.2]oct-3-yl ester (Rev atropate); 2-{1-[2-(2,3-Dihydro-benzofuran-5-yl)-ethyl]-pyrrolidin-3-yl}-2,2-diphenyl-acetamide (Darifenacin); 4-Azepan-1-yl-2,2-diphenyl-butyramide (Buzepide); 7-[3-(2-Diethylamino-acetoxy)-2-phenyl-propionyloxy]-9-ethyl-9-methyl-3-oxa-9-azonia-tricyclo[3.3.1.02,4]nonane (Oxitropium-N,N-diethylglycinate); 7-[2-(2-Diethylamino-acetoxy)-2,2-di-thiophen-2-yl-acetoxyl-9,9-dimethyl-3-oxa-9-azonia-tricyclo[3.3.1.02,4]nonane (Tiotropium-N,N-diethylglycinate); Dimethylamino-acetic acid 2-(3-diisopropylamino-1-phenyl-propyl)-4-methyl-phenyl ester (Tolterodine-N,N-dimethylglycinate); 3-[4,4-Bis-(4-fluoro-phenyl)-2-oxo-imidazolidin-1-yl]-1-methyl-1-(2-oxo-2-pyridin-2-yl-ethyl)-pyrrolidinium; 1-[1-(3-Fluoro-benzyl)-piperidin-4-yl]-4,4-bis-(4-fluoro-phenyl)-imidazolidin-2-one; 1-Cyclooctyl-3-(3-methoxy-1-aza-bicyclo[2.2.2]oct-3-yl)-1-phenyl-prop-2-yn-1-ol; 3-[2-(2-Diethylamino-acetoxy)-2,2-di-thiophen-2-yl-acetoxy]-1-(3-phenoxy-propyl)-1-azonia-bicyclo[2.2.2]octane (Aclidinium-N,N-diethylglycinate); or (2-Diethylamino-acetoxy)-di-thiophen-2-yl-acetic acid 1-methyl-1-(2-phenoxy-ethyl)-piperidin-4-yl ester; revefenacin, glycopyrronium bromide, umeclidinium bromide, tiotropium bromide, aclidinium bromide, bencycloquidium bromide.

### Mucolytic Agents

The crystalline forms provided herein may also be combined with mucolytic agents to treat both the infection and symptoms of respiratory infections. A non-limiting example of a mucolytic agent is ambroxol. Similarly, the crystalline forms may be combined with expectorants to treat both the infection and symptoms of respiratory infections. A non-limiting example of an expectorant is guaifenesin.

Nebulized hypertonic saline is used to improve immediate and long-term clearance of small airways in patients with lung diseases (Kuzik, J. Pediatrics 2007, 266). Thus, the crystalline forms provided herein may also be combined with nebulized hypertonic saline particularly when the virus infection is complicated with bronchiolitis. The combination of the crystalline forms provided herein with hypertonic saline may also comprise any of the additional agents discussed above. In one embodiment, nebulized about 3% hypertonic saline is used.

### Combination Therapy for the Treatment of Flaviviridae Virus Infections

The crystalline forms provided herein are also used in combination with other active therapeutic agents. For the treatment of Flaviviridae virus infections, preferably, the other active therapeutic agent is active against Flaviviridae virus infections.

For treatment of the dengue virus infection, non-limiting examples of the other active therapeutic agents are host cell factor modulators, such as GBV-006; fenretinide ABX-220, BRM-211; alpha-glucosidase 1 inhibitors, such as celgosivir; platelet activating factor receptor (PAFR) antagonists, such as modipafant; cadherin-5/Factor Ia modulators, such as FX-06; NS4B inhibitors, such as JNJ-8359; viral RNA splicing modulators, such as ABX-202; a NS5 polymerase inhibitor; a NS3 protease inhibitor; and a TLR modulator.

In some embodiments, the other active therapeutic agent may be a vaccine for the treatment or prevention of dengue, including but not limited to TetraVax-DV, Dengvaxia®, DPIV-001, TAK-003, live attenuated dengue vaccine, tetravalent dengue fever vaccine, tetravalent DNA vaccine, rDEN2delta30-7169; and DENV-1 PIV.

### Combination Therapy for the Treatment of Filoviridae Virus Infections

The crystalline forms provided herein are also used in combination with other active therapeutic agents. For the treatment of Filoviridae virus infections, preferably, the other active therapeutic agent is active against Filoviridae virus infections, particularly Marburg virus, Ebola virus and Cueva virus infections. Non-limiting examples of these other active therapeutic agents are: ribavirin, amiodarone, dronedarone, verapamil, Ebola Convalescent Plasma (ECP), TKM-100201, BCX4430 ((2S,3S,4R,5R)-2-(4-amino-5H-pyrrolo[3,2-d]pyrimidin-7-yl)-5-(hydroxymethyl)pyrrolidine-3,4-diol), TKM-Ebola, T-705 monophosphate, T-705 diphosphate, T-705 triphosphate, FGI-106 (1-N,7-N-bis[3-(dimethylamino)propyl]-3,9-dimethylquinolino[8,7-h]quinolone-1,7-diamine), rNAPc2, OS-2966, brincidofovir, remdesivir; RNA polymerase inhibitors, such as galidesivir, favipiravir (also known as T-705 or Avigan), JK-05; host cell factor modulators, such as GMV-006; cadherin-5/factor Ia modulators, such as FX-06; and antibodies for the treatment of Ebola, such as REGN-3470-3471-3479 and ZMapp.

Other non-limiting active therapeutic agents active against Ebola include an alpha-glucosidase 1 inhibitor, a cathepsin B inhibitor, a CD29 antagonist, a dendritic ICAM-3 grabbing nonintegrin 1 inhibitor, an estrogen receptor antagonist, a factor VII antagonist HLA class II antigen modulator, a host cell factor modulator, a Interferon alpha ligand, a neutral alpha glucosidase AB inhibitor, a niemann-Pick C1 protein inhibitor, a nucleoprotein inhibitor, a polymerase cofactor VP35 inhibitor, a Serine protease inhibitor, a tissue factor inhibitor, a TLR-3 agonist, a viral envelope glycoprotein inhibitor, and an Ebola virus entry inhibitors (NPC1 inhibitors).

In some embodiments, the other active therapeutic agent may be a vaccine for the treatment or prevention of Ebola, including but not limited to VRC-EBOADC076-00-VP, adenovirus-based Ebola vaccine, rVSV-EBOV, rVSVN4CT1-EBOVGP, MVA-BN Filo+Ad26-ZEBOV regimen, INO-4212, VRC-EBODNA023-00-VP, VRC-EBOADC069-00-VP, GamEvac-combi vaccine, SRC VB Vector, HPIV3/EboGP vaccine, MVA-EBOZ, Ebola recombinant glycoprotein vaccine, Vaxart adenovirus vector 5-based Ebola vaccine, FiloVax vaccine, GOVX-E301, and GOVX-E302.

The crystalline forms provided herein may also be used in combination with phosphoramidate morpholino oligomers (PMOs), which are synthetic antisense oligonucleotide analogs designed to interfere with translational processes by forming base-pair duplexes with specific RNA sequences. Examples of PMOs include but are not limited to AVI-7287, AVI-7288, AVI-7537, AVI-7539, AVI-6002, and AVI-6003.

The crystalline forms provided herein are also intended for use with general care provided to patients with Filoviridae viral infections, including parenteral fluids (including dextrose saline and Ringer's lactate) and nutrition, antibiotic (including metronidazole and cephalosporin antibiotics, such as ceftriaxone and cefuroxime) and/or antifungal prophylaxis, fever and pain medication, antiemetic (such as metoclopramide) and/or antidiarrheal agents, vitamin and mineral supplements (including Vitamin K and zinc sulfate), anti-inflammatory agents (such as ibuprofen), pain medications, and medications for other common diseases in the patient population, such anti-malarial agents (including artemether and artesunate-lumefantrine combination therapy), typhoid (including quinolone antibiotics, such as ciprofloxacin, macrolide antibiotics, such as azithromycin, cephalosporin antibiotics, such as ceftriaxone, or aminopenicillins, such as ampicillin), or shigellosis.

The invention will be described in greater detail by way of specific examples. The following examples are offered for illustrative purposes and are not intended to limit the invention in any manner. Those of skill in the art will readily recognize a variety of non-critical parameters, which can be changed or modified to yield essentially the same results.

### EXAMPLES

### General Procedures

XRPD patterns were collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640e) was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b.

DSC data were collected using a TA Instruments Q2000 differential scanning calorimeter. Temperature calibration was performed using NIST-traceable indium metal. The sample was placed into a Tzero aluminum DSC pan, covered with a lid pierced using a needle. The weight was then accurately recorded. A weighed aluminum pan configured as the sample pan was placed on the reference side of the cell. The sample was heated from 20° C. to 300° C. at ° C./minute.

Thermogravimetric Analysis (TGA) data were collected using a TA Instruments Q5000 thermogravimetric analyzer. Temperature calibration was performed using nickel and Alumel™. Each sample was placed in an aluminum pan and inserted into the TG furnace. The furnace was heated under a nitrogen purge. The sample was heated from ambient to 300° C. at 10° C./minute.

### Example 1. Crystalline Form Hydrate Form H1 of a Compound of Formula (I)

Crystalline form hydrate Form H1 of a compound of Formula (I) was first prepared by slurrying about 50 mg of amorphous compound of Formula (I) in about 0.4 mL of water at ambient temperature for about 2 days. The mixture was then isolated by centrifugation and the wet solids were characterized by X-Ray.

Crystalline form hydrate Form H1 of a compound of Formula (I) was also prepared by slurrying amorphous compound of Formula (I) in water. To about 100 mg of amorphous compound of Formula (I) was added about 1 mL of water. The resulting mixture was stirred at ambient temperature for about 1 day. The solid (wet cake) was filtered by centrifuge and characterized by X-ray.

Crystalline form hydrate Form H1 of a compound of Formula (I) can also be prepared by slurrying amorphous compound of Formula (I) in acetonitrile/water 2/98 (v/v) or 5/95 (v/v) mixtures. To about 50 mg of amorphous compound of Formula (I) was added about 1 mL of acetonitrile/water mixtures. The resulting mixture was stirred at ambient temperature for about 1 day. The solid (wet cake) was filtered by centrifuge and characterized by X-ray.

Crystalline form hydrate Form H1 of a compound of Formula (I) is a hydrate phase. Its XRPD pattern is shown in FIG. 1 and is characterized by Tier 1 reflections at 9.9, 15.9, 19.7 ° 2θ, but also Tier 2 at 13.3, 19.0, 21.7 ° 2θ, and Tier 3 at 17.7, 12.1, 24.4 ° 2θ. A list of 2θ peaks is provided below:

TABLE 1
XRPD peak list for crystalline form hydrate
Form H1 of a compound of Formula (I) by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 9.9 | 61 | 13.3 | 18 | 12.1 | 5 |
| 15.9 | 33 | 19.0 | 94 | 17.7 | 44 |
| 19.7 | 100 | 21.7 | 34 | 24.4 | 36 |

TABLE 2
Complete XRPD peak list for crystalline form
hydrate Form H1 of a compound of Formula (I)

| Pos. [°2θ] | Rel. Int. [%] |
|---|---|
| 9.9 | 61 |
| 10.1 | 24 |
| 10.3 | 14 |
| 12.1 | 5 |
| 13.1 | 10 |
| 13.3 | 18 |
| 15.9 | 33 |
| 17.1 | 1 |
| 17.7 | 44 |
| 18.1 | 20 |
| 19.0 | 94 |
| 19.7 | 100 |
| 20.0 | 17 |
| 20.7 | 5 |
| 21.7 | 34 |
| 21.9 | 6 |
| 23.0 | 13 |
| 24.0 | 6 |
| 24.4 | 36 |
| 24.6 | 29 |
| 24.9 | 28 |
| 26.5 | 5 |
| 27.0 | 10 |
| 27.3 | 8 |
| 27.6 | 3 |
| 28.3 | 7 |
| 29.0 | 10 |
| 29.5 | 2 |
| 30.0 | 12 |
| 31.0 | 10 |
| 31.3 | 13 |
| 31.8 | 6 |
| 32.4 | 8 |
| 32.8 | 8 |
| 33.1 | 3 |
| 33.6 | 5 |
| 34.3 | 13 |
| 34.7 | 4 |
| 34.8 | 5 |
| 35.9 | 5 |
| 36.9 | 7 |
| 37.8 | 10 |
| 38.6 | 1 |

### Example 2. Crystalline Form I of the Compound of Formula (I)

Dry solid (Form I) was obtained after wet cake (from Example 1) was subjected to vacuum drying at about 50° C.

Form I is a dehydrated phase after water is removed from Crystalline form hydrate of a compound of Formula (I) (Example 1) through drying. Its XRPD pattern is shown in FIG. 2 and is characterized by Tier 1 reflections at 10.1, 16.0, 19.8 ° 2θ, but also Tier 2 at 9.9, 13.5, 19.1 ° 2θ, and Tier 3 at 18.2, 20.1, 24.4 ° 2θ. A list of 2θ peaks is provided below:

TABLE 3
XRPD peak list for crystalline Form I
of the compound of Formula (I) by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 10.1 | 54 | 9.9 | 40 | 18.2 | 58 |
| 16.0 | 37 | 13.5 | 17 | 20.1 | 43 |
| 19.8 | 100 | 19.1 | 76 | 24.4 | 48 |

TABLE 4
Complete XRPD peak list for crystalline
Form I of the compound of Formula (I)

| Pos. [°2θ] | Rel. Int. [%] |
|---|---|
| 2.2 | 3 |
| 9.9 | 40 |
| 10.1 | 54 |
| 10.3 | 20 |
| 12.1 | 4 |
| 13.2 | 8 |
| 13.3 | 10 |
| 13.5 | 17 |
| 16.0 | 37 |
| 17.7 | 38 |
| 17.8 | 20 |
| 18.2 | 58 |
| 19.1 | 76 |
| 19.8 | 100 |
| 20.1 | 43 |
| 20.7 | 6 |
| 21.7 | 29 |
| 22.1 | 10 |
| 22.5 | 1 |
| 23.0 | 9 |
| 23.5 | 2 |
| 24.1 | 11 |
| 24.4 | 48 |
| 24.7 | 23 |
| 25.0 | 44 |
| 26.6 | 7 |
| 27.0 | 10 |
| 27.4 | 8 |
| 28.3 | 10 |
| 28.6 | 5 |
| 29.0 | 9 |
| 29.6 | 6 |
| 30.0 | 10 |
| 31.1 | 7 |
| 31.3 | 10 |
| 31.5 | 6 |
| 31.9 | 9 |
| 32.4 | 6 |
| 32.8 | 8 |
| 33.7 | 6 |
| 34.3 | 12 |
| 34.6 | 3 |
| 34.9 | 4 |
| 35.3 | 2 |
| 36.0 | 5 |
| 37.0 | 8 |
| 37.8 | 13 |
| 38.5 | 2 |
| 38.8 | 3 |

The DSC thermogram is shown in FIG. 3 and exhibits a first endothermic transition at about 151° C. followed by a second endotherm with onset at about 179° C. The TGA thermogram is shown in FIG. 4 and indicates that this phase is dehydrated (free of water).

### Example 3. Compound of Formula (I) Acetonitrile Solvate Form ACN1

Compound of Formula (I) acetonitrile solvate Form ACN1 was first prepared by slurrying amorphous compound of Formula (I) in acetonitrile. About 0.4 mL of acetonitrile was added to about 40 mg of amorphous compound of Formula (I). This resulted in a clear solution. Approximately 60 mg of amorphous compound of Formula (I) was then added to that solution. No change was observed. The mixture was stirred for about a day at ambient temperature. After that time a slurry was observed. The slurry was left to stir for another day, then it was filtered by centrifugation and the wet solids were analyzed by X-ray.

Compound of Formula (I), Acetonitrile solvate Form ACN1 is a solvated phase. Its XRPD pattern is shown in FIG. 5 and is characterized by Tier 1 reflections at 6.4, 11.8, 16.1° 2θ, but also Tier 2 at 10.0, 14.1, 20.0 ° 2θ, and Tier 3 at 15.8, 18.1, 25.5 ° 2θ. A list of 2θ peaks is provided below:

TABLE 5
XRPD peak list for compound of Formula (I)
acetonitrile solvate Form ACN1 by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.4 | 23 | 10.0 | 6 | 15.8 | 41 |
| 11.8 | 12 | 14.1 | 34 | 18.1 | 27 |
| 16.1 | 100 | 20.0 | 37 | 25.5 | 32 |

TABLE 6
Complete XRPD peak list for compound of Formula
(I) acetonitrile solvate Form ACN1

| Pos. [°2θ] | Rel. Int. [%] |
|---|---|
| 6.4 | 23 |
| 6.6 | 13 |
| 10.0 | 6 |
| 10.5 | 2 |
| 11.8 | 12 |
| 12.6 | 6 |
| 13.3 | 4 |
| 13.8 | 18 |
| 14.1 | 34 |
| 14.4 | 32 |
| 15.8 | 41 |
| 16.1 | 100 |
| 16.5 | 8 |
| 17.9 | 19 |
| 18.1 | 27 |
| 18.3 | 15 |
| 18.7 | 5 |
| 19.0 | 10 |
| 19.3 | 13 |
| 20.0 | 37 |
| 20.2 | 22 |
| 21.0 | 8 |
| 21.5 | 13 |
| 22.1 | 9 |
| 23.1 | 21 |
| 23.8 | 13 |
| 24.5 | 26 |
| 25.1 | 10 |
| 25.5 | 32 |
| 27.0 | 11 |
| 28.1 | 7 |
| 29.5 | 3 |
| 30.4 | 4 |
| 30.8 | 9 |
| 31.7 | 4 |
| 32.4 | 5 |
| 33.8 | 2 |
| 34.4 | 6 |
| 35.3 | 3 |
| 36.2 | 5 |
| 37.4 | 6 |
| 38.9 | 4 |

### Example 4. Compound of Formula (I) Acetonitrile Solvate Form ACN2

Compound of Formula (I) acetonitrile solvate Form ACN2 was prepared from amorphous compound of Formula (I) in acetonitrile. To about 340 mg of amorphous compound of Formula (I) was added about 1 mL of acetonitrile. The resulting mixture was heated to about 50° C. to afford a clear solution. The solution was cooled to ambient temperature. Small amount of seeds from Form ACN1 (Example 3) was added to the clear solution and the mixture was left stirring for about 1 day. The resulting solid (wet cake, ACN2) was filtered by centrifuge and characterized by X-ray.

Compound of Formula (I) acetonitrile solvate Form ACN2 is a solvated phase. Its XRPD pattern is shown in FIG. 6 and is characterized by Tier 1 reflections at 7.9, 16.2, 23.8° 2θ, but also Tier 2 at 7.1, 11.6, 17.5 ° 2θ, and Tier 3 at 18.3, 25.2, 26.3 ° 2θ. A list of 2θ peaks is provided below:

TABLE 7
XRPD peak list for compound of Formula (I)
Acetonitrile solvate Form ACN2 by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 7.9 | 100 | 7.1 | 3 | 18.3 | 6 |
| 16.2 | 61 | 11.6 | 2 | 25.2 | 8 |
| 23.8 | 15 | 17.5 | 8 | 26.3 | 18 |

TABLE 8
Complete XRPD peak list for compound of Formula
(I), acetonitrile solvate Form ACN2

| Pos. [°2θ] | Rel. Int. [%] |
|---|---|
| 7.1 | 3 |
| 7.9 | 100 |
| 11.6 | 2 |
| 13.6 | 1 |
| 13.9 | 1 |
| 14.5 | 1 |
| 15.7 | 3 |
| 16.2 | 61 |
| 17.5 | 8 |
| 17.9 | 1 |
| 18.3 | 6 |
| 18.8 | 0 |
| 19.3 | 1 |
| 20.3 | 1 |
| 20.7 | 2 |
| 21.1 | 2 |
| 21.8 | 4 |
| 22.7 | 3 |
| 23.8 | 15 |
| 25.2 | 8 |
| 26.3 | 18 |
| 26.9 | 2 |
| 27.7 | 2 |
| 28.6 | 2 |
| 29.1 | 3 |
| 29.9 | 5 |
| 31.2 | 1 |
| 32.7 | 1 |
| 33.9 | 1 |
| 34.5 | 1 |
| 34.9 | 1 |
| 35.4 | 0 |
| 36.6 | 1 |
| 37.2 | 1 |
| 37.8 | 1 |
| 38.8 | 1 |

### Example 5. Compound of Formula (I) Acetonitrile Solvate Form ACN3

Compound of Formula I acetonitrile solvate Form ACN3 was first prepared by mixing a comparable amount (approximately 200 mg) of Form ACN1 (Example 3) slurry and Form ACN2 (Example 4) slurry in acetonitrile. The mixture was stirred under ambient condition with a magnetic stirrer for at least 6 hours. The resulting solid (wet cake, ACN3) was filtered by centrifuge and characterized by X-ray.

Compound of Formula I acetonitrile solvate Form ACN3 was also be prepared by holding Acetonitrile Form ACN2 slurry at ambient condition without agitation for 3 weeks. The resulting solid (wet cake, ACN3) was filtered by centrifuge and characterized by X-ray.

Compound of Formula I acetonitrile solvate Form ACN3 is a solvated phase. Its XRPD pattern is shown in FIG. 7 and is characterized by Tier 1 reflections at 6.3, 7.9, 16.2° 2θ, but also Tier 2 at 7.1, 13.1, 18.0 ° 2θ, and Tier 3 at 15.3, 19.3, 21.3 ° 2θ. A list of 2θ peaks is provided below:

TABLE 9
XRPD peak list for compound of Formula I
acetonitrile solvate Form ACN3 by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.3 | 100 | 7.1 | 70 | 15.3 | 8 |
| 7.9 | 56 | 13.1 | 9 | 19.3 | 17 |
| 16.2 | 52 | 18.0 | 26 | 21.3 | 11 |

TABLE 10
Complete XRPD peak list for compound of
Formula I acetonitrile solvate Form ACN3

| Pos. [°2θ] | Rel. Int. [%] |
|---|---|
| 6.3 | 100 |
| 7.1 | 70 |
| 7.9 | 56 |
| 10.5 | 0 |
| 11.5 | 2 |
| 12.6 | 3 |
| 13.1 | 9 |
| 13.6 | 1 |
| 14.2 | 8 |
| 14.6 | 4 |
| 15.3 | 8 |
| 15.6 | 2 |
| 16.2 | 52 |
| 16.8 | 10 |
| 17.5 | 1 |
| 18.0 | 26 |
| 18.3 | 2 |
| 18.9 | 5 |
| 19.3 | 17 |
| 19.9 | 2 |
| 21.3 | 11 |
| 22.4 | 2 |
| 22.7 | 2 |
| 23.2 | 3 |
| 23.8 | 2 |
| 24.1 | 8 |
| 24.8 | 3 |
| 25.2 | 9 |
| 25.6 | 6 |
| 26.3 | 17 |
| 27.3 | 13 |
| 27.7 | 4 |
| 28.6 | 5 |
| 29.9 | 6 |
| 30.9 | 2 |
| 31.9 | 1 |
| 32.7 | 1 |
| 33.8 | 1 |
| 34.8 | 1 |
| 37.0 | 1 |
| 37.5 | 1 |
| 38.8 | 2 |

### Example 6. Compound of Formula (I) Form II

Compound of Formula (I) Form II was obtained after wet cake (Form ACN3, Example 5) was subjected to vacuum drying at about 50° C.

Compound of Formula (I) Form II is a desolvated phase after acetonitrile is removed from Form ACN3 (Example 5) through drying. Its XRPD pattern is shown in FIG. 8 and is characterized by Tier 1 reflections at 7.9, 16.2, 23.8 ° 2θ, but also Tier 2 at 13.6, 18.3, 26.3 ° 2θ, and Tier 3 at 17.5, 25.2, 29.9 ° 2θ. A list of 2θ peak is shown below.

TABLE 11
XRPD peak list for compound of Formula (I) Form II by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 7.9 | 100 | 13.6 | 1 | 17.5 | 2 |
| 16.2 | 60 | 18.3 | 4 | 25.2 | 9 |
| 23.8 | 17 | 26.3 | 18 | 29.9 | 5 |

TABLE 12
Complete XRPD peak list for compound of Formula (I) Form II

| Pos. [°2θ] | Rel. Int.[%] |
|---|---|
| 7.1 | 0 |
| 7.9 | 100 |
| 11.6 | 1 |
| 13.6 | 1 |
| 13.9 | 1 |
| 14.5 | 1 |
| 15.8 | 3 |
| 16.2 | 60 |
| 17.5 | 2 |
| 18.0 | 1 |
| 18.3 | 4 |
| 19.3 | 1 |
| 20.3 | 0 |
| 20.7 | 1 |
| 21.1 | 1 |
| 21.6 | 1 |
| 21.8 | 2 |
| 22.1 | 0 |
| 22.7 | 3 |
| 23.5 | 3 |
| 23.8 | 17 |
| 25.2 | 9 |
| 26.3 | 18 |
| 26.6 | 1 |
| 27.7 | 2 |
| 28.6 | 2 |
| 29.1 | 2 |
| 29.9 | 5 |
| 31.3 | 0 |
| 31.9 | 0 |
| 32.7 | 1 |
| 33.8 | 1 |
| 34.5 | 0 |
| 34.9 | 1 |
| 35.5 | 0 |
| 37.2 | 0 |
| 37.8 | 1 |
| 38.2 | 1 |
| 38.8 | 1 |

The DSC thermogram of compound of Formula (I) Form II is shown in FIG. 9 and exhibits a first endothermic transition at about 125° C. followed by an immediate exotherm, indicating a melting/recrystallization transition. A second endotherm is observed with onset at about 180° C. The TGA thermogram is of compound of Formula (I) Form II shown in FIG. 10 and indicates that this phase is desolvated (free of solvent).

### Example 7. Compound of Formula (I) Solvate in Other Organic Solvents and Other Desolvated Forms

Examples 3-6 are examples of compound of Formula (I) freebase forming multiple solvated forms in a particular process solvent (acetonitrile). Solvated forms have been observed in other common organic solvents such as ethyl acetate and ethanol. The labile nature of the solvents in the solvated forms means the solvents can be readily removed by drying operation (vacuum or temperature or both). Each solvated form could lead to a specific desolvated form. For instance, Form II (Example 6) is the corresponding desolvated form of ACN3 (Example 5).

Due to the unique chemical structure of the compound of Formula (I), the molecule has a propensity to form many forms of hydrates and solvates, as well as the corresponding desolvated and dehydrated forms. These include, but are not limited to, organic solvents such as methanol, ethanol, isopropanol, 1-butanol, 2-butanol (e.g. (S)-2-butanol and (R)-2-butanol), acetone, acetonitrile, ethyl acetate, propyl acetate (e.g. isopropyl acetate), butyl acetate, methyl t-butyl ether (MTBE), tetrahydrofuran, toluene, 1-butanone (methyl ethyl ketone), 2-methyl tetrahydrofuran, heptane (e.g. n-heptane), cyclohexane, cyclopentyl methyl ether, dichloromethane, N,N-dimethylacetamide, N,N-dimethylformamide, ethyleneglycol, hexane (e.g. n-hexane), propylene glycol, methyl butyl ketone, methylcyclohexane, methylisobutylketone, N-methylpyrrolidone, t-butyl alcohol, acetic acid, anisole, dimethyl sulfoxide, isobutyl acetate, methyl acetate, 2-methyl-1-propanol, ethyl ether, ethyl formate, formic acid, pentane (e.g. n-pentane), 1-pentanol, triethylamine.

The unique chemical structure of the compound of Formula (I) also implies the likelihood of forming numerous cocrystals with coformers, as well as crystalline salt forms if the pKa differential between compound of Formula (I) and the counterion is sufficient to enable proton transfer (salt formation). These may include, but are not limited to, acids such as acetic acid, adipic acid, alginic acid, ascorbic acid, aspartic acid, benzenesulfonic acid, benzoic acid, camphoric acid, camphor-10-sulfonic acid, citric acid, cyclamic acid, dodecylsulfuric acid, ethane-1,2-disulfoic acid, ethanesulfonic acid, 2-hydroxy-ethanesulfonic acid, formic acid, fumaric acid, galactaric acid, gentisic acid, D-glucoheptonic acid, D-gluconic acid, D-glucuronic acid, glutamic acid, glutaric acid, glycolic acid, hippuric acid, hydrochloric acid, isobutyric acid, lactic acid, lauric acid, maleic acid, malic acid, malonic acid, mandelic acid, methanesulfonic acid, mucic acid, naphthalene-1,5-disolfonic acid, naphthalene-2-sulfonic acid, nicotinic acid, oleic acid, orotic acid, oxalic acid, palmitic acid, pamoic acid, phosphoric acid, propionic acid, pyroglutamic acid, salicylic acid, sebacic acid, stearic acid, succinic acid, sulfuric acid, tartaric acid, thiocyanic acid, and p-toluenesulfonic acid.

### Example 8. Compound of Formula I, HCl Salt, Form I

Compound of Formula I, HCl salt, Form I was first prepared by dissolving about 50 mg of crystalline Form III of Formula (I) freebase in about 0.4 mL of acetonitrile. About one molar equivalent of HCl in isopropyl alcohol was added and the resulting clear solution was stirred at ambient temperature. A slurry was observed upon stirring the mixture for about a day. The solids were isolated by centrifugation and dried in a vacuum oven at 50° C. for about a day.

Compound of Formula I, HCl salt, Form I was also prepared by dissolving about 50 mg of crystalline Form III of Formula I freebase in about 0.4 mL of acetonitrile. About one molar equivalent of aqueous concentrated HCl was added and the resulting clear solution was stirred at ambient temperature. A slurry was observed upon stirring the mixture for about a day. The solids were isolated by centrifugation and dried in a vacuum oven at 50° C. for about a day.

Compound of Formula I, HCl salt, Form I was also prepared by slurrying about 200 mg of crystalline Form III of Formula I freebase in about 2 mL of acetonitrile. About one molar equivalent of aqueous concentrated HCl was added and the slurry was stirred at ambient temperature for about a day. The slurry was then filtered under vacuum and washed with about 0.5 mL acetonitrile. The wet solids were dried in a vacuum oven at 50° C. for about a day.

Compound of Formula I, HCl salt, Form I was a crystalline phase. Its XRPD pattern is shown in FIG. 11 and is characterized by Tier 1 reflections at 5.8, 11.5, 18.0 ° 2θ, but also Tier 2 at 14.1, 20.0, 27.0 ° 2θ, and Tier 3 at 13.8, 22.1, 29.5 ° 2θ.

TABLE 13
XRPD peak list for compound of Formula
I, HCl salt, Form I by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 5.8 | 48 | 14.1 | 40 | 13.8 | 22 |
| 11.5 | 100 | 20.0 | 43 | 22.1 | 32 |
| 18.0 | 67 | 27.0 | 33 | 29.5 | 21 |

TABLE 14
Complete XRPD peak list for compound
of Formula I, HCl salt, Form I

| Pos. [°2Th.] | Rel. Int. [%] |
|---|---|
| 5.8 | 48 |
| 11.5 | 100 |
| 12.1 | 5 |
| 13.8 | 22 |
| 14.1 | 40 |
| 15.0 | 17 |
| 16.3 | 11 |
| 16.7 | 16 |
| 17.3 | 7 |
| 18.0 | 67 |
| 18.7 | 11 |
| 19.2 | 11 |
| 20.0 | 43 |
| 21.6 | 8 |
| 22.1 | 32 |
| 23.0 | 5 |
| 23.5 | 14 |
| 24.3 | 33 |
| 24.7 | 28 |
| 25.4 | 6 |
| 25.9 | 5 |
| 26.5 | 5 |
| 27.0 | 33 |
| 27.4 | 15 |
| 28.3 | 5 |
| 28.8 | 11 |
| 29.2 | 12 |
| 29.5 | 21 |
| 31.2 | 7 |
| 32.0 | 14 |
| 32.9 | 5 |
| 34.8 | 7 |
| 36.4 | 8 |
| 37.0 | 3 |
| 37.7 | 3 |

The DSC thermogram is shown in FIG. 12 and exhibits an endothermic transition at about 123° C. The TGA thermogram is shown in FIG. 4 FIG. 13 and indicates that this phase is solvated.

### Example 9. Compound of Formula (I), Oxalate Salt, Form I

Compound of Formula (I), oxalate salt, Form I was first prepared by dissolving about 50 mg of crystalline Form III of Formula (I) freebase in about 0.4 mL acetonitrile. Approximately one molar equivalent of oxalic acid was added, and the solution was stirred for about a day at ambient conditions. This resulted in a slurry. The solids were isolated by centrifugation and dried in a vacuum oven at 50° C. for about a day.

Compound of Formula (I), oxalate salt, Form I was a crystalline phase. Its XRPD pattern is shown in FIG. 14 and is characterized by Tier 1 reflections at 6.6, 17.2, 29.5 ° 2θ, but also Tier 2 at 6.9, 13.8, 21.8, ° 2θ, and Tier 3 at 10.2, 20.7, 27.4 ° 2θ. The DSC thermogram is shown in FIG. 15 and exhibits an endothermic transition with onset at about 113° C. The TGA thermogram is shown in FIG. 16 and indicates that this phase is solvated.

TABLE 15
XRPD peak list for Compound of Formula (I), Oxalate Salt, Form I

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.6 | 100 | 6.9 | 53 | 10.2 | 23 |
| 17.2 | 32 | 13.8 | 32 | 20.7 | 21 |
| 29.5 | 12 | 21.8 | 24 | 27.4 | 11 |

TABLE 16
Complete XRPD peak list for Compound
of Formula (I), Oxalate Salt, Form I

| Pos. [°2Th.] | Rel. Int. [%] |
|---|---|
| 6.6 | 100 |
| 6.9 | 53 |
| 8.1 | 6 |
| 9.8 | 7 |
| 10.2 | 32 |
| 11.1 | 8 |
| 12.6 | 8 |
| 13.8 | 23 |
| 14.9 | 5 |
| 15.6 | 18 |
| 16.4 | 15 |
| 17.2 | 32 |
| 18.3 | 11 |
| 18.9 | 7 |
| 19.6 | 19 |
| 19.7 | 18 |
| 20.7 | 21 |
| 21.1 | 12 |
| 21.8 | 24 |
| 22.3 | 12 |
| 22.7 | 11 |
| 24.3 | 11 |
| 26.1 | 7 |
| 26.4 | 12 |
| 27.4 | 11 |
| 28.6 | 5 |
| 29.5 | 12 |
| 31.6 | 4 |
| 33.5 | 2 |
| 34.4 | 2 |

### Example 10. Compound of Formula (I), Oxalate Salt, Form II

Compound of Formula (I), oxalate salt, Form II was first prepared by dissolving about 200 mg of crystalline Form III of Formula (I) freebase in about 2 mL acetonitrile. Approximately one molar equivalent of oxalic acid was added, and the solution was stirred for about four days at ambient conditions. The resulting slurry was filtered under vacuum and dried in a vacuum oven at 50° C. for about a day to obtain Compound of Formula (I), Oxalate salt, Form II.

Compound of Formula (I), oxalate salt, Form II was a crystalline phase. Its XRPD pattern is shown in FIG. 17 and is characterized by Tier 1 reflections at 3.7, 10.3, 26.4 ° 2θ, but also Tier 2 at 7.3, 18.2, 28.0 ° 2θ, and Tier 3 at 6.4, 15.0, 19.6 ° 2θ. The DSC thermogram is shown in FIG. 18 and exhibits an endothermic transition with onset at about 130° C. The TGA thermogram is shown in FIG. 19 and indicates that this phase is solvated.

TABLE 17
XRPD peak list for Compound of Formula
(I), Oxalate Salt, Form II by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.7 | 100 | 7.3 | 18 | 6.4 | 10 |
| 10.3 | 52 | 18.2 | 33 | 15.0 | 27 |
| 26.4 | 33 | 28.0 | 21 | 19.6 | 30 |

TABLE 18
Complete XRPD peak list for Compound
of Formula (I), Oxalate Salt, Form II

| Pos. [°2Th.] | Rel. Int. [%] |
|---|---|
| 3.7 | 100 |
| 6.4 | 10 |
| 7.3 | 18 |
| 8.1 | 7 |
| 10.3 | 52 |
| 13.1 | 20 |
| 14.5 | 9 |
| 15.0 | 27 |
| 15.4 | 26 |
| 16.3 | 15 |
| 16.6 | 26 |
| 17.2 | 6 |
| 18.2 | 33 |
| 19.0 | 13 |
| 19.6 | 30 |
| 19.9 | 9 |
| 20.8 | 14 |
| 21.4 | 14 |
| 21.9 | 8 |
| 22.2 | 29 |
| 23.1 | 18 |
| 24.5 | 6 |
| 25.6 | 8 |
| 26.0 | 8 |
| 26.4 | 33 |
| 27.4 | 7 |
| 28.0 | 21 |
| 29.5 | 12 |
| 29.9 | 8 |
| 30.1 | 5 |
| 32.2 | 5 |
| 34.1 | 4 |
| 36.9 | 5 |
| 38.5 | 4 |

### Example 11: Compound of Formula (I), Maleate Salt, MeCN Solvate I; Compound of Formula (I), Maleate Salt, MeCN Solvate II; Compound of Formula (I), Maleate Salt, Form I; Compound of Formula (I), Maleate Salt, Form II; and Compound of Formula (I), Maleate Salt, Form III

Compound of Formula (I), maleate salt, Form I was first prepared by dissolving about 50 mg of crystalline Form III of Formula (I) freebase in about 0.4 mL acetonitrile. Approximately one molar equivalent of maleic acid was added, and a slurry precipitated immediately. The slurry was stirred for a day and about 0.2 mL acetonitrile was then added to make the slurry mobile. The slurry was sampled for XRPD (maleate salt, MeCN, solvate I), and the wet solids were dried in a vacuum oven at 50° C. for about four days to obtain compound of Formula (I), maleate salt, Form I. The remaining slurry was stirred at ambient conditions for about five days. The slurry was then sampled for XRPD (maleate salt, MeCN, solvate II), and the wet solids were dried in a vacuum oven at 50° C. for about a day to obtain compound of Formula (I), maleate salt, Form II.

Compound of Formula (I), maleate salt, Form II was also prepared by slurrying about 1.5 g of material of Formula (I) freebase (purified by flash chromatography using dichloromethane/methanol as eluents) in about 15 mL of acetonitrile. Approximately one molar equivalent of maleic acid was added, and the slurry was stirred for about a day. The slurry was sampled for X-ray and the wet solids were dried to obtain Compound of Formula (I), maleate salt, Form II. The remaining slurry was isolated by vacuum filtration. The solids were washed with about 1.5 mL acetonitrile and dried in a vacuum oven at 50° C. for about a day to obtain compound of Formula (I), maleate salt, Form III.

The XRPD pattern of compound of Formula (I), maleate salt, MeCN solvate I is shown in FIG. 28 and is characterized by Tier 1 reflections at 3.5, 7.0, 20.9 ° 2θ, but also Tier 2 at, 3.7, 7.3, 22.0 ° 2θ.

The XRPD pattern of Compound of Formula (I), maleate salt, Form I is shown in FIG. 20 and is characterized by Tier 1 reflections at 3.9, 7.8, 19.7 ° 2θ, but also Tier 2 at, 11.8, 23.7 ° 2θ.

Compound of Formula (I), maleate salt, MeCN solvate II is a crystalline phase. Its XRPD pattern is shown in FIG. 29 and is characterized by Tier 1 reflections at 3.4, 6.8, 25.2 ° 2θ, but also Tier 2 at 3.7, 14.8, 27.6 ° 2θ, and Tier 3 at 16.8, 20.5, 26.7 ° 2θ.

Compound of Formula (I), maleate salt, Form II is a crystalline phase. Its XRPD pattern is shown in FIG. 21 and is characterized by Tier 1 reflections at 3.9, 7.7, 16.9 ° 2θ, but also Tier 2 at 10.4, 18.0, 25.9 ° 2θ, and Tier 3 at 15.8, 19.2, 26.8 ° 2θ.

Compound of Formula (I), maleate salt, Form III is a crystalline phase. Its XRPD pattern is shown in FIG. 22 and is characterized by Tier 1 reflections at 3.8, 7.6, 19.2 ° 2θ, but also Tier 2 at 11.5, 16.8, 23.1 ° 2θ, and Tier 3 at 10.4, 15.1, 27.5 ° 2θ. The DSC thermogram is shown in FIG. 23 and exhibits an endothermic transition with onset at about 165° C. The TGA thermogram is shown in FIG. 24 and indicates that this phase is solvated.

TABLE 19
XRPD peak list for Compound of Formula (I),
Maleate Salt, MeCN solvate I by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.5 | 100 | 3.7 | 50 | | |
| 7.0 | 8 | 7.3 | 4 | | |
| 20.9 | 2 | 22.0 | 3 | | |

TABLE 20
Complete XRPD peak list for Compound of
Formula (I), Maleate Salt, MeCN solvate I

| Pos. [°2Th.] | Rel. Int. [%] |
|---|---|
| 3.5 | 100 |
| 3.7 | 50 |
| 7.0 | 8 |
| 7.3 | 4 |
| 11.0 | 1 |
| 18.4 | 1 |
| 20.9 | 2 |
| 22.0 | 3 |

TABLE 21
XRPD peak list for Compound of Formula
(I), Maleate Salt, Form I

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.9 | 100 | 11.8 | 2 | | |
| 7.8 | 12 | 23.7 | 2 | | |
| 19.7 | 8 | | | | |

TABLE 22
Complete XRPD peak list for Compound
of Formula (I), Maleate Salt, Form I

| Pos. [°2Th.] | Rel. Int. [%] |
|---|---|
| 3.9 | 100 |
| 7.8 | 12 |
| 11.8 | 2 |
| 19.7 | 8 |
| 23.7 | 2 |

TABLE 23
XRPD peak list for Compound of Formula (I),
Maleate Salt, MeCN solvate II by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.4 | 100 | 3.7 | 24 | 16.8 | 8 |
| 6.8 | 15 | 14.8 | 6 | 20.5 | 9 |
| 25.2 | 15 | 27.6 | 15 | 26.7 | 13 |

TABLE 24
Complete XRPD peak list for Compound of Formula
(I), Maleate Salt, Maleate Salt, MeCN solvate II

| Pos. [°2Th.] | Rel. Int. [%] |
|---|---|
| 3.4 | 100 |
| 3.7 | 24 |
| 6.8 | 15 |
| 11.0 | 3 |
| 11.5 | 2 |
| 12.0 | 3 |
| 12.5 | 3 |
| 14.8 | 6 |
| 15.7 | 6 |
| 16.2 | 7 |
| 16.8 | 8 |
| 18.1 | 7 |
| 20.5 | 9 |
| 23.1 | 8 |
| 25.2 | 15 |
| 25.9 | 12 |
| 26.7 | 13 |
| 27.6 | 15 |
| 28.7 | 9 |
| 29.8 | 5 |

TABLE 25
XRPD peak list for Compound of Formula
(I), Maleate Salt, Form II by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.9 | 100 | 10.4 | 3 | 15.8 | 5 |
| 7.7 | 15 | 18.0 | 8 | 19.2 | 10 |
| 16.9 | 12 | 25.9 | 12 | 26.8 | 10 |

TABLE 26
Complete XRPD peak list for Compound
of Formula (I), Maleate Salt, Form II

| Pos. [°2Th.] | Rel. Int. [%] |
|---|---|
| 3.9 | 100 |
| 7.7 | 15 |
| 10.4 | 3 |
| 11.7 | 3 |
| 15.1 | 5 |
| 15.8 | 5 |
| 16.9 | 12 |
| 18.0 | 8 |
| 19.2 | 10 |
| 20.8 | 7 |
| 23.2 | 4 |
| 25.9 | 12 |
| 26.8 | 10 |
| 27.9 | 8 |

TABLE 27
XRPD peak list for Compound of Formula
(I), Maleate Salt, Form III

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 3.8 | 100 | 11.5 | 4 | 10.4 | 1 |
| 7.6 | 17 | 16.8 | 7 | 15.1 | 2 |
| 19.2 | 10 | 23.1 | 7 | 27.5 | 2 |

TABLE 28
Complete XRPD peak list for Compound of
Formula (I), Maleate Salt, Form III

| Pos. [°2Th.] | Rel. Int. [%] |
|---|---|
| 3.8 | 100 |
| 7.6 | 17 |
| 10.4 | 1 |
| 11.5 | 4 |
| 15.1 | 2 |
| 15.8 | 2 |
| 16.8 | 7 |
| 19.2 | 10 |
| 20.7 | 2 |
| 23.1 | 7 |
| 24.1 | 2 |
| 24.9 | 1 |
| 25.6 | 2 |
| 26.5 | 2 |
| 27.5 | 2 |
| 28.7 | 1 |
| 31.3 | 1 |

### Example 12. Compound of Formula (I) Maleate Form IV

Compound of Formula (I), maleate salt, Form IV was first prepared by dissolving about 200 mg of crystalline Form III of Formula (I) freebase in about 2 mL acetonitrile. Approximately one molar equivalent of maleic acid was added, and a slurry formed. The slurry was stirred at ambient conditions for about three days. About 1 mL of acetonitrile was added to make the slurry mobile. The slurry was stirred at ambient conditions for about a day and filtered under vacuum. The wet solids were dried in a vacuum oven at 50° C. for about a day to obtain Compound of Formula (I), maleate salt, Form IV.

Compound of Formula (I), maleate salt, Form IV is a crystalline phase. Its XRPD pattern is shown in FIG. 25 and is characterized by Tier 1 reflections at 8.8, 15.0, 25.5 ° 2θ, but also Tier 2 at 5.4, 21.4, 30.3 ° 2θ, and Tier 3 at 14.0, 17.6, 35.7 ° 2θ. The DSC thermogram is shown in FIG. 26 and exhibits an endothermic transition with onset at about 175° C. The TGA thermogram is shown in FIG. 27 and indicates that this phase is desolvated.

TABLE 29
XRPD peak list for Compound of Formula
(I), maleate salt, Form IV by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 8.8 | 100 | 5.4 | 7 | 14.0 | 9 |
| 15.0 | 61 | 21.4 | 23 | 17.6 | 21 |
| 25.5 | 36 | 30.3 | 10 | 35.7 | 9 |

TABLE 30
Complete XRPD peak list for Compound
of Formula (I), maleate salt, Form IV

| Pos. [°2Th.] | Rel. Int. [%] |
|---|---|
| 5.4 | 7 |
| 8.8 | 100 |
| 12.8 | 6 |
| 14.0 | 9 |
| 15.0 | 61 |
| 16.2 | 2 |
| 16.9 | 4 |
| 17.6 | 21 |
| 18.0 | 0 |
| 18.5 | 7 |
| 19.8 | 4 |
| 20.0 | 7 |
| 21.4 | 23 |
| 22.4 | 3 |
| 22.8 | 17 |
| 23.4 | 10 |
| 25.5 | 36 |
| 26.1 | 4 |
| 26.6 | 2 |
| 27.1 | 3 |
| 28.1 | 4 |
| 29.2 | 3 |
| 29.9 | 9 |
| 30.3 | 10 |
| 30.8 | 8 |
| 32.5 | 7 |
| 33.0 | 6 |
| 33.8 | 2 |
| 34.5 | 5 |
| 35.7 | 9 |
| 37.0 | 4 |
| 37.5 | 3 |
| 38.0 | 2 |
| 39.0 | 1 |

### Example 13. Compound of Formula (I), Mesylate Salt, IPA Solvate and Compound of Formula (I), Mesylate Salt, Hydrate

Compound of Formula (I), mesylate salt, hydrate was first prepared by dissolving about 50 mg of Form III Compound of Formula (I) freebase in about 0.4 mL of acetonitrile. Approximately one molar equivalent of methanesulfonic acid was added, and the solution was stirred at ambient conditions for about a day. The solution remained clear. The acetonitrile was removed by evaporation and about 0.4 mL ethanol was added. The solution remained clear after stirring at ambient conditions for about a day. The ethanol was then removed by evaporation and about 0.3 mL of isopropyl alcohol was added. The solution was stirred at ambient conditions for about three days and a slurry was obtained. The resulting slurry was sampled for X-ray (mesylate IPA solvate). The wet solids were dried in a vacuum oven at 50° C. for about a day to obtain compound of Formula (I), mesylate salt, hydrate.

Compound of Formula (I), mesylate salt, hydrate was also prepared by slurrying about 100 mg of crystalline Form III of Formula (I) freebase in about 1 mL of isopropyl alcohol. Approximately one molar equivalent of methanesulfonic acid was added. The resulting slurry was stirred at ambient conditions for about a day. The slurry was then sampled for X-ray to obtain the mesylate IPA solvate. The wet solids were dried in a vacuum oven at 50° C. for about three days to obtain Compound of Formula (I), mesylate salt, hydrate.

Compound of Formula (I), mesylate salt, IPA solvate is a crystalline phase. Its XRPD pattern is shown in FIG. 30 and is characterized by Tier 1 reflections at 7.5, 18.2, 25.0 ° 2θ, but also Tier 2 at 8.7, 19.7, 29.7 ° 2θ, and Tier 3 at 9.8, 16.4, 27.2 ° 2θ.

Compound of Formula (I), mesylate salt, hydrate is a crystalline phase. Its XRPD pattern is shown in FIG. 31 and is characterized by Tier 1 reflections at 8.7, 17.7, 24.8 ° 2θ, but also Tier 2 at 4.4, 19.4, 28.2 ° 2θ, and Tier 3 at 13.1, 21.1, 30.2 ° 2θ. The DSC thermogram is shown in FIG. 32 and exhibits an endothermic transition with onset at about 111° C. The TGA thermogram is shown in FIG. 33 and indicates that this phase is hydrated.

TABLE 31
XRPD peak list for Compound of Formula
(I), Mesylate Salt, Hydrate by tiers

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 8.7 | 100 | 4.4 | 19 | 13.1 | 10 |
| 17.7 | 51 | 19.4 | 18 | 21.1 | 15 |
| 24.8 | 17 | 28.2 | 12 | 30.2 | 10 |

TABLE 32
Complete XRPD peak list for Compound of
Formula (I), Mesylate Salt, Hydrate

| Pos. [°2Th.] | Rel. Int. [%] |
|---|---|
| 2.4 | 7 |
| 4.4 | 19 |
| 8.7 | 100 |
| 10.5 | 1 |
| 10.9 | 3 |
| 13.1 | 10 |
| 13.5 | 3 |
| 14.0 | 3 |
| 17.2 | 7 |
| 17.7 | 51 |
| 18.3 | 2 |
| 18.8 | 2 |
| 19.4 | 18 |
| 19.5 | 11 |
| 20.6 | 2 |
| 20.9 | 5 |
| 21.2 | 15 |
| 21.7 | 7 |
| 24.0 | 11 |
| 24.2 | 5 |
| 24.8 | 17 |
| 25.5 | 7 |
| 26.6 | 8 |
| 26.9 | 6 |
| 27.5 | 9 |
| 27.7 | 11 |
| 28.2 | 12 |
| 29.1 | 3 |
| 29.4 | 3 |
| 30.2 | 10 |
| 30.8 | 6 |
| 31.1 | 2 |
| 31.8 | 1 |
| 33.2 | 7 |
| 34.1 | 6 |
| 34.4 | 7 |
| 35.3 | 1 |
| 36.4 | 2 |
| 37.9 | 3 |
| 38.7 | 2 |
| 39.3 | 1 |

TABLE 33
XRPD peak list for Compound of Formula
(I), Mesylate salt, IPA Solvate

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 7.5 | 100 | 8.7 | 11 | 9.8 | 6 |
| 18.2 | 28 | 19.7 | 26 | 16.4 | 10 |
| 25.0 | 13 | 29.7 | 6 | 27.2 | 7 |

TABLE 34
Complete XRPD peak list for Formula
(I), Mesylate salt, IPA Solvate

| Pos. [°2Th.] | Rel. Int. [%] |
|---|---|
| 3.8 | 2 |
| 4.3 | 2 |
| 7.5 | 100 |
| 8.7 | 11 |
| 9.8 | 6 |
| 13.3 | 3 |
| 15.0 | 5 |
| 16.4 | 10 |
| 17.0 | 5 |
| 17.3 | 7 |
| 17.7 | 6 |
| 18.2 | 28 |
| 18.8 | 3 |
| 19.7 | 26 |
| 20.1 | 3 |
| 21.0 | 7 |
| 21.6 | 12 |
| 21.7 | 9 |
| 22.8 | 4 |
| 23.1 | 6 |
| 24.0 | 3 |
| 24.6 | 3 |
| 25.0 | 13 |
| 26.2 | 4 |
| 26.7 | 4 |
| 27.2 | 7 |
| 27.6 | 3 |
| 28.5 | 7 |
| 29.7 | 6 |
| 30.2 | 4 |
| 32.5 | 4 |
| 33.4 | 2 |
| 34.1 | 3 |
| 34.9 | 4 |
| 36.5 | 3 |
| 38.0 | 1 |
| 38.9 | 2 |

### Example 14. Compound of Formula (I), Mesylate Salt, Form I

Compound of Formula (I), mesylate salt, Form I was first prepared by charging about 5 gr of crystalline Form IV of Formula (I) freebase to a glass reactor and adding about 50 mL of isopropyl alcohol. Approximately one molar equivalent of methanesulfonic acid was added, and a clear solution was obtained after stirring for approximately 30 minutes at ambient conditions. The solution was seeded with a small amount of mesylate hydrate and left stirring at ambient conditions for about a day. This resulted in a slurry. Approximately 0.1 molar equivalent of methanesulfonic acid was added after 3 days, and the slurry was stirred for about a day at ambient conditions. The slurry was filtered under vacuum and washed with the mother liquors and about 2 mL of isopropyl alcohol. The solids were dried in a vacuum oven at 50° C. for about a day to obtain Compound of Formula (I) mesylate Form I.

Compound of Formula (I), mesylate salt, Form I is a crystalline phase. Its XRPD pattern is shown in FIG. 34 and is characterized by Tier 1 reflections at 6.3, 13.8, 21.6 ° 2θ, but also Tier 2 at 12.7, 19.1 24.5 ° 2θ, and Tier 3 at 14.6, 23.4, 29.7 ° 2θ. The DSC thermogram is shown in FIG. 35 and exhibits an endothermic transition with onset at about 163° C. The TGA thermogram is shown in FIG. 36 and indicates that this phase is desolvated.

TABLE 35
XRPD peak list for Compound of Formula
(I), Mesylate Salt, Form I

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.3 | 74 | 12.7 | 46 | 14.6 | 23 |
| 13.8 | 86 | 19.1 | 35 | 23.4 | 16 |
| 21.6 | 100 | 24.5 | 33 | 29.7 | 15 |

TABLE 36
Complete XRPD peak list for Compound
of Formula (I), Mesylate Salt, Form I

| Pos. [°2Th.] | Rel. Int. [%] |
|---|---|
| 6.3 | 74 |
| 10.5 | 2 |
| 11.9 | 1 |
| 12.7 | 46 |
| 13.8 | 86 |
| 14.2 | 8 |
| 14.6 | 23 |
| 15.5 | 1 |
| 16.2 | 1 |
| 17.1 | 20 |
| 18.8 | 23 |
| 19.1 | 35 |
| 20.1 | 4 |
| 20.4 | 11 |
| 21.1 | 15 |
| 21.3 | 40 |
| 21.6 | 100 |
| 22.3 | 8 |
| 22.5 | 16 |
| 22.8 | 11 |
| 23.4 | 16 |
| 23.8 | 25 |
| 24.5 | 33 |
| 25.7 | 5 |
| 25.9 | 10 |
| 26.5 | 9 |
| 27.5 | 4 |
| 27.9 | 12 |
| 28.8 | 4 |
| 29.7 | 15 |
| 30.2 | 4 |
| 30.5 | 8 |
| 32.0 | 17 |
| 32.3 | 4 |
| 32.7 | 5 |
| 33.5 | 6 |
| 34.5 | 6 |
| 34.6 | 10 |
| 35.3 | 7 |
| 36.1 | 2 |
| 36.5 | 6 |
| 36.8 | 4 |
| 38.2 | 3 |
| 39.5 | 2 |

### Example 15. Compound of Formula (I), Form III

Compound of Formula (I), Form III was prepared by drying crystalline Form ACN1 in the oven at about 50° C. for about a day.

### Characterization

Compound of Formula (I), Form III is a crystalline phase. Its XRPD pattern is shown in FIG. 37 and is characterized by Tier 1 reflections at 6.5, 16.0, 19.9 ° 2θ, but also Tier 2 at 13.9, 18.2, 23.3 ° 2θ, and Tier 3 at 10.4, 19.2, 25.4 ° 2θ.

TABLE 37
XRPD peak list for compound of Formula (I), Form III

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.5 | 100 | 13.9 | 14 | 10.4 | 3 |
| 16.0 | 83 | 18.2 | 18 | 19.2 | 13 |
| 19.9 | 56 | 23.3 | 11 | 25.4 | 9 |

TABLE 38
Complete XRPD peak list for compound of Formula (I), Form III

| Pos. [°2Th.] | Rel. Int. [%] |
|---|---|
| 6.1 | 5 |
| 6.5 | 100 |
| 9.9 | 2 |
| 10.4 | 3 |
| 11.9 | 2 |
| 12.7 | 2 |
| 13.1 | 4 |
| 13.9 | 14 |
| 14.2 | 6 |
| 16.0 | 83 |
| 16.4 | 11 |
| 18.2 | 18 |
| 19.1 | 6 |
| 19.2 | 13 |
| 19.5 | 6 |
| 19.9 | 56 |
| 20.0 | 6 |
| 20.7 | 5 |
| 20.9 | 14 |
| 21.3 | 10 |
| 22.2 | 2 |
| 23.0 | 10 |
| 23.3 | 11 |
| 23.6 | 7 |
| 24.3 | 8 |
| 25.0 | 10 |
| 25.4 | 9 |
| 26.2 | 3 |
| 26.9 | 8 |
| 27.3 | 5 |
| 27.8 | 6 |
| 28.0 | 5 |
| 29.3 | 2 |
| 30.2 | 2 |
| 30.4 | 3 |
| 30.7 | 8 |
| 31.2 | 1 |
| 31.6 | 3 |
| 32.5 | 4 |
| 33.1 | 1 |
| 33.6 | 3 |
| 34.3 | 3 |
| 35.3 | 2 |
| 35.8 | 1 |
| 36.1 | 2 |
| 37.3 | 4 |
| 38.0 | 2 |
| 38.4 | 3 |
| 38.8 | 3 |

### Example 16. Compound of Formula (I), Form IV

Compound of Formula (I), Form IV was prepared by dissolving about 50 mg of crystalline Form III of Formula (I), freebase in about 0.5 mL of acetonitrile, methanol, acetone, methyl ethyl ketone, or 2-methyltetrahydrofuran. The solutions were saturated by adding 50-100 mg of crystalline Form III of Formula (I), freebase and left to stir for about 19-20 days after becoming slurries. The slurries were sampled for XRPD and dried in a vacuum oven at 50° C. for about a day to obtain Compound of Formula (I), Form IV.

### Characterization

Compound of Formula (I), Form IV is a crystalline phase. Its XRPD pattern is shown in FIG. 38 and is characterized by Tier 1 reflections at 6.7°, 10.2°, and 18.0 ° 2θ, but also Tier 2 at 9.0°, 16.5°, and 18.6 ° 2θ, and Tier 3 at 10.9°, 20.1°, and 25.6 ° 2θ. The DSC thermogram is shown in FIG. 39 and exhibits an endothermic event with an onset at about 25° C. A second endothermic transition is observed at about 119° C. followed by an immediate exotherm, indicating a melting/recrystallization transition. A third endothermic transition is observed, with an onset at about 179° C. The TGA thermogram is shown in FIG. 40 and indicates that this phase is solvated.

TABLE 39
XRPD peak list for compound of Formula (I), Form IV

| Tier 1 | | Tier 2 | | Tier 3 | |
|---|---|---|---|---|---|
| Position | Relative | Position | Relative | Position | Relative |
| °2θ | intensity (%) | °2θ | intensity (%) | °2θ | intensity (%) |
| 6.7 | 100 | 9.0 | 87 | 10.9 | 8 |
| 10.2 | 40 | 16.5 | 15 | 20.1 | 20 |
| 18.0 | 35 | 18.6 | 31 | 25.6 | 8 |

TABLE 40
Complete XRPD peak list for compound of Formula (I), Form IV

| Pos. [°2Th.] | Rel. Int. [%] |
|---|---|
| 6.7 | 100 |
| 9.0 | 87 |
| 10.2 | 40 |
| 10.9 | 8 |
| 15.5 | 4 |
| 16.3 | 11 |
| 16.5 | 15 |
| 17.4 | 2 |
| 18.0 | 35 |
| 18.6 | 31 |
| 19.1 | 6 |
| 19.8 | 3 |
| 20.1 | 20 |
| 20.4 | 7 |
| 20.7 | 8 |
| 21.3 | 5 |
| 22.2 | 6 |
| 23.0 | 7 |
| 23.4 | 4 |
| 24.0 | 5 |
| 24.5 | 8 |
| 25.6 | 8 |
| 26.0 | 2 |
| 27.0 | 4 |
| 27.6 | 4 |
| 28.0 | 8 |
| 28.4 | 4 |
| 28.9 | 4 |
| 29.4 | 3 |
| 30.0 | 1 |
| 31.3 | 5 |
| 31.5 | 5 |
| 35.5 | 3 |
| 37.8 | 3 |

All references, including publications, patents, and patent documents are incorporated by reference herein, as though individually incorporated by reference. The present disclosure provides reference to various embodiments and techniques. However, it should be understood that many variations and modifications may be made while remaining within the spirit and scope of the present disclosure. The description is made with the understanding that it is to be considered an exemplification of the claimed subject matter and is not intended to limit the appended claims to the specific embodiments illustrated.

1. A crystalline form of a compound of Formula (I):

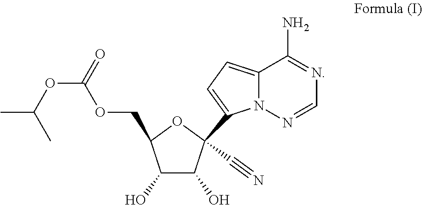 [SMILES: CC(C)OC(=O)OC[C@H]1O[C@@](C#N)(c2ccc3c(N)ncnn23)[C@H](O)[C@@H]1O]

2. The crystalline form of claim 1, wherein the crystalline form is Form I characterized by an XRPD pattern comprising 2θ-reflections (±0.2 degrees 2θ) at 10.1°, 16.0°, and 19.8°.

3. (canceled)

4. The crystalline form of claim 2, wherein the XRPD pattern comprises 2θ-reflections (±0.2 degrees 2θ) at 9.9°, 10.1°, 13.5°, 16.0°, 19.1°, and 19.8°.

5.-7. (canceled)

8. The crystalline form of claim 1, wherein the crystalline form is characterized by a differential scanning calorimetry pattern comprising a first endothermic transition at about 151° C. followed by a second endotherm with onset at about 179° C.

9. (canceled)

10. (canceled)

11. The crystalline form of claim 1, wherein the crystalline form is Form II characterized by an XRPD pattern comprising 2θ-reflections (±0.2 degrees 2θ) at 7.9°, 16.2°, and 23.8°.

12. (canceled)

13. The crystalline form of claim 11, wherein the XRPD comprises 2θ-reflections (±0.2 degrees 2θ) at 7.9°, 13.6°, 16.2°, 18.3°, 23.8°, and 26.3°.

14.-16. (canceled)

17. The crystalline form claim 11, wherein the crystalline form is characterized by a differential scanning calorimetry pattern comprising a first endothermic transition at about 125° C. followed by an immediate exotherm, and a second endotherm with onset at about 180° C.

18. (canceled)

19. (canceled)

20. The crystalline form of claim 1, wherein the crystalline form is Form III characterized by an XRPD pattern comprising 2θ-reflections (±0.2 degrees 2θ) at 6.5°, 16.0°, and 19.9°.

21. (canceled)

22. The crystalline form of claim 20, wherein the XRPD comprises 2θ-reflections (±0.2 degrees 2θ) at 6.5°, 13.9°, 16.0°, 18.2°, 19.9°, and 23.3°.

23.-27. (canceled)

28. The crystalline form of claim 1, wherein the crystalline form is Form IV characterized by an XRPD pattern comprising 2θ-reflections (±0.2 degrees 2θ) at 6.7°, 10.2°, and 18.0°.

29. (canceled)

30. The crystalline form of claim 28, wherein the XRPD comprises 2θ-reflections (±0.2 degrees 2θ) at 6.7°, 9.0°, 10.2°, 16.5°, 18.0° and 18.6°.

31.-33. (canceled)

34. The crystalline form of claim 28,
wherein the crystalline form is characterized by a differential scanning calorimetry pattern comprising an endothermic event with an onset at about 25° C., a second endothermic transition at about 119° C., and a third endothermic transition with an onset at about 179° C.

35.-38. (canceled)

39. A solvated crystalline form of a compound of Formula (I):

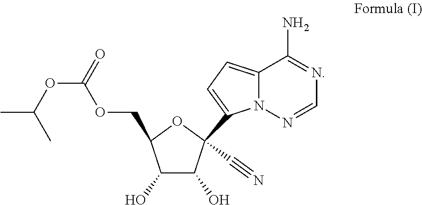 [SMILES: CC(C)OC(=O)OC[C@H]1O[C@@](C#N)(c2ccc3c(N)ncnn23)[C@H](O)[C@@H]1O]

40. (canceled)

41. The solvated crystalline form of claim 39, wherein the solvated crystalline form is an ethyl acetate solvate, an ethanol solvate or an acetonitrile solvate.

42. The solvated crystalline form of claim 39, wherein the crystalline form is an acetonitrile solvate.

43. The solvated crystalline form of claim 42, wherein the crystalline form is acetonitrile solvate Form ACN1 characterized by an XRPD pattern comprising 2θ-reflections (±0.2 degrees 2θ) at 6.4°, 11.8°, and 16.1°.

44. (canceled)

45. The solvated crystalline form of claim 43, wherein the XRPD comprises 2θ-reflections (±0.2 degrees 2θ) at 6.4°, 10.0°, 11.8°, 14.1°, 16.1°, and 20.0°.

46.-48. (canceled)

49. The solvated crystalline form of claim 42, wherein the crystalline form is acetonitrile solvate Form ACN2 characterized by an XRPD pattern comprising 2θ-reflections (±0.2 degrees 2θ) at 7.9°, 16.2°, and 23.8°.

50. (canceled)

51. The solvated crystalline form of claim 49, wherein the XRPD comprises 2θ-reflections (±0.2 degrees 2θ) at 7.1°, 7.9°, 11.6°, 16.2°, 17.5°, and 23.8°.

52.-54. (canceled)

55. The solvated crystalline form of claim 42, wherein the crystalline form is acetonitrile solvate Form ACN3 characterized by an XRPD pattern comprising 2θ-reflections (±0.2 degrees 2θ) at 6.3°, 7.9°, and 16.2°.

56. (canceled)

57. The solvated crystalline form of claim 55, wherein the XRPD comprises 2θ-reflections (±0.2 degrees 2θ) at 6.3°, 7.1°, 7.9°, 13.1°, 16.2°, and 18.0°.

58.-60. (canceled)

61. The solvated crystalline form of claim 39, wherein the solvated crystalline form is a hydrate.

62. The solvated crystalline form of claim 61, wherein the crystalline form is hydrate Form H1 characterized by an XRPD pattern comprising 2θ-reflections (±0.2 degrees 2θ) at 9.9°, 15.9°, and 19.7°.

63. (canceled)

64. The solvated crystalline form of claim 62, wherein the XRPD pattern comprises 2θ-reflections (±0.2 degrees 2θ) at 9.9°, 13.3°, 15.9°, 19.0°, 19.7°, and 21.7°.

65.-67. (canceled)

68. A salt of a compound of Formula (I):

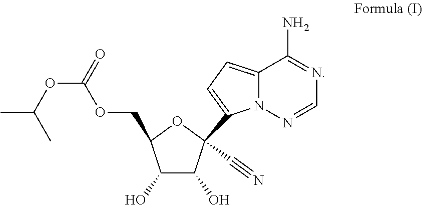 [SMILES: CC(C)OC(=O)OC[C@H]1O[C@@](C#N)(c2ccc3c(N)ncnn23)[C@H](O)[C@@H]1O]

69. The salt of claim 68, wherein the salt is a HCl salt, an oxalate salt, a maleate salt, or a mesylate salt of the compound of Formula I.

70.-72. (canceled)

73. A crystalline form of a salt of a compound of Formula (I):

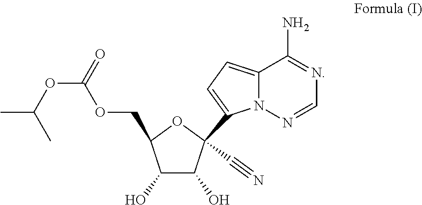 [SMILES: CC(C)OC(=O)OC[C@H]1O[C@@](C#N)(c2ccc3c(N)ncnn23)[C@H](O)[C@@H]1O]

74. The crystalline form of claim 73, wherein the crystalline form is a crystalline form of a HCl salt of the compound of Formula I.

75. The crystalline form of claim 74, characterized by an XRPD pattern comprising 2θ-reflections (±0.2 degrees 2θ) at 5.8°, 11.5°, and 18.0°.

76. (canceled)

77. The crystalline form of claim 75, wherein the XRPD pattern comprises one, 2θ-reflections (±0.2 degrees 2θ) at 5.8°, 11.5°, 14.1°, 18.0°, 20.0°, and 27.0°.

78.-80. (canceled)

81. The crystalline form of claim 74, wherein the crystalline form is characterized by a differential scanning calorimetry pattern comprising an endothermic transition at about 123° C.

82. (canceled)

83. (canceled)

84. The crystalline form of claim 73, wherein the crystalline form is a crystalline form of an oxalate salt of the compound of Formula I.

85. The crystalline form of claim 84, characterized by an XRPD pattern comprising 2θ-reflections (±0.2 degrees 2θ) at 6.6°, 17.2°, and 29.5°.

86. (canceled)

87. The crystalline form of claim 85, wherein the XRPD pattern comprises one, 2θ-reflections (±0.2 degrees 2θ) at 6.6°, 6.9°, 13.8°, 17.2°, 21.8°, and 29.5°.

88.-90. (canceled)

91. The crystalline form of claim 84, wherein the crystalline form is characterized by a differential scanning calorimetry pattern comprising an endothermic transition with onset at about 113° C.

92. (canceled)

93. (canceled)

94. The crystalline form of claim 84, characterized by an XRPD pattern comprising 2θ-reflections (±0.2 degrees 2θ) at 3.7°, 10.3°, and 26.4°.

95. (canceled)

96. The crystalline form of claim 94, wherein the XRPD pattern comprises one, 2θ-reflections (±0.2 degrees 2θ) at 3.7°, 7.3°, 10.3°, 18.2°, 26.4°, and 28.0°.

97.-99. (canceled)

100. The crystalline form of claim 94, wherein the crystalline form is characterized by a differential scanning calorimetry pattern comprising an endothermic transition with onset at about 130° C.

101. (canceled)

102. (canceled)

103. The crystalline form of claim 73, wherein the crystalline form is a crystalline form of a maleate salt of the compound of Formula I.

104. The crystalline form of claim 103, characterized by an XRPD pattern comprising 2θ-reflections (±0.2 degrees 2θ) at 3.9°, 7.8°, and 19.7°.

105. (canceled)

106. The crystalline form of claim 104 or 105, wherein the XRPD pattern comprises 2θ-reflections (±0.2 degrees 2θ) at 3.9°, 7.8°, 11.8°, 19.7°, and 23.7°.

107. (canceled)

108. The crystalline form of claim 103, characterized by an XRPD pattern comprising 2θ-reflections (±0.2 degrees 2θ) at 3.9°, 7.7°, and 16.9°.

109. (canceled)

110. The crystalline form of claim 108, wherein the XRPD pattern comprises 2θ-reflections (±0.2 degrees 2θ) at 3.9°, 7.7°, 10.4°, 16.9°, 18.0°, and 25.9°.

111.-113. (canceled)

114. The crystalline form of claim 103, characterized by an XRPD pattern comprising 2θ-reflections (±0.2 degrees 2θ) at 3.8°, 7.6°, and 19.2°.

115. (canceled)

116. The crystalline form of claim 114 or 115, wherein the XRPD pattern comprises 2θ-reflections (±0.2 degrees 2θ) at 3.8°, 7.6°, 11.5°, 16.8°, 19.2°, and 23.1°.

117.-119. (canceled)

120. The crystalline form of claim 114, wherein the crystalline form is characterized by a differential scanning calorimetry pattern comprising an endothermic transition with onset at about 165° C.

121. (canceled)

122. (canceled)

123. The crystalline form of claim 103, characterized by an XRPD pattern comprising 2θ-reflections (±0.2 degrees 2θ) at 8.8°, 15.0°, and 25.5°.

124. (canceled)

125. The crystalline form of claim 123 or 1211, wherein the XRPD pattern comprises 2θ-reflections (±0.2 degrees 2θ) at 5.4°, 8.8°, 15.0°, 21.4°, 25.5°, and 30.3°.

126.-128. (canceled)

129. The crystalline form of claim 123, wherein the crystalline form is characterized by a differential scanning calorimetry pattern comprising an endothermic transition with onset at about 175° C.

130. (canceled)

131. (canceled)

132. The crystalline form of claim 103, wherein the crystalline form is a solvate.

133. The crystalline form of claim 132, wherein the crystalline form is an acetonitrile solvate.

134. The crystalline form of claim 133, characterized by an XRPD pattern comprising 2θ-reflections (±0.2 degrees 2θ) at 3.5°, 7.0°, and 20.9°.

135. (canceled)

136. The crystalline form of claim 134 or 135, wherein the XRPD pattern comprises 2θ-reflections (±0.2 degrees 2θ) at 3.5°, 3.7°, 7.0°, 7.3°, 20.9°, and 22.0°.

137. (canceled)

138. The crystalline form of claim 133, characterized by an XRPD pattern comprising 2θ-reflections (±0.2 degrees 2θ) at 3.4°, 6.8°, and 25.2°.

139. (canceled)

140. The crystalline form of claim 138, wherein the XRPD pattern comprises 2θ-reflections (±0.2 degrees 2θ) at 3.4°, 3.7°, 6.8°, 14.8°, 25.2° and 27.6°.

141.-143. (canceled)

144. The crystalline form of claim 73, wherein the crystalline form is a crystalline form of a mesylate salt of the compound of Formula I.

145. The crystalline form of claim 144, wherein the crystalline form is a solvate.

146. The crystalline form of claim 145, wherein the crystalline form is an isopropyl alcohol solvate.

147. The crystalline form of claim 146, characterized by an XRPD pattern comprising 2θ-reflections (±0.2 degrees 2θ) at 7.5°, 18.2°, and 25.0°.

148. (canceled)

149. The crystalline form of claim 147, wherein the XRPD pattern comprises 2θ-reflections (±0.2 degrees 2θ) at 7.5°, 8.7°, 18.2°, 19.7°, and 29.7°.

150.-152. (canceled)

153. The crystalline form of claim 145, wherein the crystalline form is a hydrate.

154. The crystalline form of claim 153, characterized by an XRPD pattern comprising 2θ-reflections (±0.2 degrees 2θ) at 8.7°, 17.7°, and 24.8°.

155. (canceled)

156. The crystalline form of claim 154 or 155, wherein the XRPD pattern comprises 2θ-reflections (±0.2 degrees 2θ) at 4.4°, 8.7°, 17.7°, 19.4°, 24.8°, and 28.2°.

157.-159. (canceled)

160. The crystalline form of claim 153, wherein the crystalline form is characterized by a differential scanning calorimetry pattern comprising an endothermic transition with onset at about 111° C.

161. (canceled)

162. (canceled)

163. The crystalline form of claim 144, characterized by an XRPD pattern comprising 2θ-reflections (±0.2 degrees 2θ) at 6.3°, 13.8°, and 21.6°.

164. (canceled)

165. The crystalline form of claim 163, wherein the XRPD pattern comprises 2θ-reflections (±0.2 degrees 2θ) at 6.3°, 12.7°, 13.8°, 19.1°, 21.6°, and 24.5°.

166.-168. (canceled)

169. The crystalline form of claim 163, wherein the crystalline form is characterized by a differential scanning calorimetry pattern comprising an endothermic transition with onset at about 163° C.

170.-227. (canceled)
